(19) United States
(12) Patent Application Publication
(10) Pub. No.: US 2026/0080977 A1
(43) Pub. Date: Mar. 19, 2026

(54) BIOLOGICAL PROGRAMMING LANGUAGE

(71) Applicant: EvolutionaryScale, PBC, New York, NY (US)

(72) Inventors: Salvatore J. Candido, San Francisco, CA (US); Thomas F. Hayes, San Francisco, CA (US); Alexander W. Rives, Brooklyn, NY (US)

(21) Appl. No.: 19/243,433

(22) Filed: Jun. 19, 2025

Related U.S. Application Data
(60) Provisional application No. 63/662,331, filed on Jun. 20, 2024.
(60) Provisional application No. 63/663,494, filed on Jun. 24, 2024.

(51) Int. Cl. G16B 40/00; G16B 50/00

(52) U.S. Cl. CPC G16B 40/00; G16B 50/00

(57) ABSTRACT

A biological programming specification that identifies at least one protein design condition in accordance with a biological programming language is received. A machine learning model is used to convert the biological programming specification to a model input format version for a biological reasoning model. The model input format version is used as a conditioning input for the biological reasoning model to generate a protein design having the at least one protein design condition.

### CROSS REFERENCE TO OTHER APPLICATIONS

This application claims priority to U.S. Provisional Patent Application No. 63/663,494 entitled BIOLOGICAL PROGRAMMING LANGUAGE filed Jun. 24, 2024 which is incorporated herein by reference for all purposes. This application claims priority to U.S. Provisional Patent Application No. 63/662,331 entitled GENERATIVE MULTIMODAL PROTEIN LANGUAGE MODEL filed Jun. 20, 2024 which is incorporated herein by reference for all purposes.

### BACKGROUND OF THE INVENTION

Biological objects such as proteins can be described by their multiple properties such as by atomic makeup, function, and physical structure. For example, proteins are commonly described by an amino acid sequence, a physical structure, and exhibited functions. Existing techniques analyze these properties which can be used to generate and design proteins including proteins with particular structure requirements and constraints. Specifying these requirements and constraints, such as for the generation and design of biological molecules including proteins, is fundamentally hard due to the biological complexity of the subject. Therefore, there is a compelling need for a solution for describing biological requirements, such as via a high-level biological programming language, that is high-level, modular, and easily expressed.

### BRIEF DESCRIPTION OF THE DRAWINGS

Various embodiments of the invention are disclosed in the following detailed description and the accompanying drawings.

FIG. 1 is a block diagram illustrating an embodiment of a biological language reasoning platform that includes the ability to predict and generate biological language results using a biological language model.

FIG. 2 is a block diagram illustrating an embodiment of a biological language model service for generating and predicting biological language reasoning results.

FIG. 3 is a block diagram illustrating an embodiment of a multi-track biological language reasoning model.

FIG. 4 is a block diagram illustrating an embodiment of a biological language model module capable of generating and predicting biological language reasoning results.

FIG. 5 is a block diagram illustrating an embodiment of a multi-track biological protein language model module capable of generating and predicting biological protein language reasoning results including structure results.

FIG. 6 is a block diagram illustrating an embodiment of a transformer block with a geometric attention mechanism of a multi-track biological language model.

FIG. 7 is a flow chart illustrating an embodiment of a process for performing biological language reasoning using a biological language reasoning model.

FIG. 8 is a flow chart illustrating an embodiment of a process for training a biological language reasoning model.

FIG. 9 is a flow chart illustrating an embodiment of a process for training a biological protein language model.

FIG. 10 is a flow chart illustrating an embodiment of a process for applying a biological language reasoning model to a biological language search query.

FIG. 11 is a flow chart illustrating an embodiment of a process for applying a biological protein language model to a biological protein language search query.

FIG. 12 is a block diagram illustrating an embodiment of a tokenizer for biological structure.

FIG. 13 is a block diagram illustrating an embodiment of a tokenizer for biological structure.

FIG. 14 is a block diagram illustrating an embodiment of a structure encoder of a biological structure tokenizer.

FIG. 15 is a block diagram illustrating an embodiment of a geometric reasoning block of a biological structure tokenizer.

FIG. 16 is a flow chart illustrating an embodiment of a process for training a biological structure tokenizer.

FIG. 17 is a flow chart illustrating an embodiment of a process for tokenizing biological structure.

FIG. 18 is a flow chart illustrating an embodiment of a process for tokenizing a biological protein structure.

FIG. 19 is a block diagram illustrating an embodiment of a geometric attention block for performing geometric attention on biological structure.

FIG. 20 is a block diagram illustrating an embodiment of a geometric attention block for performing geometric attention on biological structure.

FIG. 21 is a flow chart illustrating an embodiment of a process for performing geometric attention on biological structure.

FIG. 22 is a flow chart illustrating an embodiment of a process for determining a direction attention result on biological structure.

FIG. 23 is a flow chart illustrating an embodiment of a process for determining a distance attention result on biological structure.

FIG. 24 is a flow chart illustrating an embodiment of a process for determining a geometric attention result on biological structure from direction and distance attention results.

FIG. 25 is a flow chart illustrating an embodiment of a process for training a biological language reasoning model to support a biological programming language.

FIG. 26 is a flow chart illustrating an embodiment of a process for converting biological design conditions to conditioning input for a biological reasoning model using a biological programming language.

FIG. 27 is a flow chart illustrating an embodiment of a process for interfacing with a biological language reasoning model using a biological language program.

FIG. 28 is a flow chart illustrating an embodiment of a process for compiling a biological programming specification into model-compatible conditioning inputs.

FIG. 29 is a flow chart illustrating an embodiment of a process for compiling a biological programming specification into model-compatible conditioning inputs using a large language model (LLM).

FIG. 30 is a diagram illustrating an embodiment of an excerpt from a biological programming specification for generating a protein design using a biological reasoning model.

FIG. 31 is a diagram illustrating an embodiment of an excerpt from a biological programming specification for generating a protein design using a biological reasoning model.

FIG. 32 is a functional diagram illustrating a programmed computer system for performing biological language reasoning.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

A biological programming language for interfacing with biological language reasoning models is disclosed. The disclosed biological programming language (also referred to as a biological language programming language or biological reasoning programming language) and related techniques and systems allow for the easy and modular expression of biological requirements and constraints such as design and generation requirements and constraints. For example, the disclosed biological programming language can be used with the disclosed biological language reasoning model and/or other biological reasoning models for designing biological molecules, including proteins, that meet desired design properties such as symmetry, functionality, and stability requirements, among other conditions. Additional design goals and constraints may include properties related to developability and immunogenicity. For example, using the disclosed techniques and platforms, design goals can specify developability and immunogenicity properties/characteristics of a candidate protein, particularly in the context of protein therapeutics. Among its advantages, the biological programming language allows a user to specify the desired and required conditions via a high-level interface that is reusable, modular, and easily accessible. For example, once design goals are expressed using the biological programming language, the same biological program can be compiled to target one or more different biological reasoning models for biological design generation.

In some embodiments, the biological programming language is first compiled into an intermediate representation before being converted into the input format version required by a target biological reasoning model. For instance, design conditions specified using a biological programming language can be compiled into a structured intermediate form, which is then adapted to the input requirements of one or more different biological reasoning models. In some embodiments, the same biological program can be compiled to input conditions for two or more different protein folding models, allowing their predicted outputs to be evaluated against one another. In some embodiments, the biological programming language can further support a generative design workflow that integrates multiple biological reasoning models, such as through a joint optimization framework. For example, a protein design process may be expressed using a biological programming language wherein the output of one model is used as the input for another, enabling iterative refinement of candidate designs. Additionally, the compilation process may include validation logic that detects invalid or conflicting constraints, such as conditions that require physically unrealistic geometries or infeasible motif placements. In some embodiments, when such conflicts are identified, the compiler can suggest revisions or enhancements to the biological program to improve design feasibility and/or to guide the user toward successful biological design outcomes.

In some embodiments, the compilation process is further utilized to implement additional safeguards such as restrictions for certain biological designs. For example, programmatic constraints and conditions aimed at intentionally or inadvertently producing harmful or prohibited functions can be disallowed during the compilation stage. In some embodiments, the safeguards are ethical and security filters that notify the user of and/or prevent the generation of potentially harmful or unauthorized biological outputs. For example, the compiler may implement a screening layer that detects and flags high-risk constraints, such as functionality associated with pathogenic proteins, neurotoxins, or viral entry domains. In various embodiments, the compiler can reject the specification, sanitize it, require manual authorization, log the compilation request, and/or intervene in an another appropriate manner. By integrating these filters directly into the compilation process, and in advance of model inference, the approach prevents unsafe or ethically problematic instructions from ever reaching the generative backend, thereby enabling responsible deployment of protein design systems in compliance with biosecurity standards.

In some embodiments, the compilation process is performed using a large language model (LLM). For example, an LLM can be prompted to convert the biological programming language into conditioning input for a biological reasoning model. In particular embodiments, an LLM is utilized to convert the intermediate representation of a biological programming language into the specific conditioning input requirements of a model. By utilizing an LLM-based compilation process and a universal intermediate representation, new biological reasoning models and their respective conditioning input formats can be rapidly supported. The biological programming language combined with the LLM-based compiler provides for flexible, scalable, and model-agnostic translation of high-level biological programs into inputs for generative biological reasoning models. The disclosed process allows for long-term adaptability and support for diverse biological design workflows.

In certain embodiments, the compilation process performed using an LLM can dynamically translate a biological programming language specification into conditioning inputs suitable for one or more different biological reasoning models. The LLM can receive, as input, a high-level biological program that describes the desired design constraints and conditions, such as structural elements, functional properties, stability properties, functional motifs, symmetry, solvent accessibility, developability properties, and/or immunogenicity properties, among others. The LLM interprets the syntax and semantics of the biological programming specification using, for example, contextual understanding and in-context learning. In various embodiments, the LLM is prompted with details of the biological programming language, documentation, example programs, and corresponding example input and/or input format requirements for the target biological reasoning model, among other contextual details. The LLM can generate an intermediate representation of the provided biological programming specification, and based on the target biological reasoning model (e.g., a protein folding model, a diffusion-based model, a text diffusion LLM, an autoregressive decoder LLM, a structure-based foundation model, or a multi-track biological language reasoning model, among others), the LLM converts the intermediate representation into model-specific conditioning inputs. For example, different model input formats may require a per-residue token input format, span-level annotations, global conditioning vectors or tensors, and/or geometry-based embeddings, among others. In various embodiments, the compilation process enables the same biological program to be compiled into distinct model input format versions depending on the architecture and input interface of the backend generative biological model. Furthermore, by modifying the prompt structure and/or fine-tuning the LLM on new intermediate representation-to-conditioning data pairs, the LLM-based compiler and its support for new model types and input modalities can be improved with minimal manual rule engineering. The disclosed architecture and approach significantly improve the flexibility, usefulness, and application of biological programs, allowing them to adapt to evolving generative models and maintain long-term compatibility across different and changing biological design workflows.

In various embodiments, the biological programming language includes a visual component that allows the specified constraints and conditions to be visualized. For example, a syntax tree representing a biological language program can be visualized with non-terminal and terminal nodes. The different nodes can be displayed along with their associated conditions, such as their programmed properties, requirements, and/or constraints. In particular embodiments, the visualization is performed using the intermediate representation. For example, the biological programming language can be visualized independent of the target biological model, and model-specific support can be included as appropriate. In various embodiments, the disclosed biological programming language is applicable for conditioning a biological language reasoning model including the disclosed multi-track biological language reasoning model. For example, the disclosed biological language reasoning model is trained to support a biological programming language and to accept one or more conditioning tracks generated from biological language programs.

In some embodiments, a biological programming language specification is created that supports describing biological conditions. The supported conditions can be selected for interfacing and controlling a biological language reasoning model such as for the design and generation of biological objects including proteins. Example conditions supported by the biological programming language can relate to functional properties, stability properties, developability properties, immunogenicity properties, symmetries of structure, symmetries of amino acid sequences, structure templates including relative positions of atoms within a subset of residues, portions of a biological object which are surface exposed, secondary structure on portions of proteins, hydrophobic amino acid properties of proteins including the quantity of hydrophobic amino acids, the globularity of a portion of a protein, functional specificity for a portion of a biological object, molecular interactions and interfaces including protein to protein interactions and/or interfaces, small molecular interactions, DNA/RNA interaction and binding properties, motif and active site scaffolding, and/or post translational modifications, among other conditions. In various embodiments, the biological programming language specification provides a standardized approach for specifying the conditions and each of the conditions can have one or more corresponding program or programming language keywords for describing the context associated with the condition.

In some embodiments, a biological language reasoning model is trained to receive as input one or more specified conditions supported by the biological programming language specification via a biological language program. For example, the biological language reasoning model may be a multi-track biological language reasoning model and the conditions (including requirements and constraints) specified by the biological language program can correspond to one or more input tracks to the trained model. In various embodiments, the received biological language program is created using the high-level language defined by the biological programming language specification and compiled to generate the one or more conditioning tracks or input conditions used as input to the biological language reasoning model. For example, via a compilation process, the high-level language and interface supported by a biological programming language and understood by users can be converted to a conditioning track understood by the multi-track biological language reasoning model. In various embodiments, the high-level language is converted to the specific input format version used by a target biological reasoning model, regardless of whether the model has a specific input conditioning track.

In some embodiments, the biological language reasoning model is trained to support the biological programming language specification and the training can be implemented as pretraining and/or post-training objectives. In some embodiments, for each supported condition and/or for a given portion of the biological programming language specification, a reference biological language program is created along with corresponding examples of biological objects such as proteins exhibiting the described condition(s). In various embodiments, the identified examples correspond to appropriate outputs for the associated biological language program. The identified examples can be used as target results associated with the reference biological language programs and, along with the reference programs, are used to create a training data set. The identified examples, target results, and/or training data can be created by mining data sources of natural proteins such as public and/or private data stores and/or by utilizing and/or creating synthetic data. In various embodiments, the model is trained using the reference biological language programs as conditioning inputs and their corresponding target results as outputs. Once trained, at inference time the biological language reasoning model is able to predict a desired biological object or objects based on a provided biological language program. In some embodiments, the format of the biological language program understood by the biological language reasoning model is in the form of a tensor and/or conditioning track. For example, a biological language program including each of the reference biological language programs used for training can be compiled into a conditioning track or tensor used by the biological language reasoning model.

In some embodiments, a biological language program conforming to a biological programming language specification is received. For example, a biological language program written to a biological programming language specification describes conditions such as requirements and constraints for a target biological object (or objects) such as a target protein. The conditions specified by the biological language program can include conditions associated with structure, function, stability, developability, immunogenicity, interaction sites, post translational modification, surface exposure, secondary structure, hydrophobic properties, and globularity properties, among others. In some embodiments, one or more conditions specified by the biological language program are identified. For example, the biological language program is analyzed and/or compiled to identify conditions described by the biological language program. The biological language program may be compiled into an intermediate representation such as a syntax tree with non-terminal and terminal nodes. The identified conditions can be associated with the nodes of the syntax tree. In some embodiments, the conditions are applied hierarchically, and conditions associated with non-terminal nodes are applied to all child nodes of the non-terminal node. In some embodiments, using the identified one or more conditions, conditioning input is generated for a biological language reasoning model. For example, the identified conditions are used to generate conditioning input such as input for a conditioning track of a biological language reasoning model. In various embodiments, the biological language reasoning model receives the conditioning input for performing biological language reasoning including for predicting biological results. For example, by using a biological language program, a user can specify conditions for designing and generating a protein using a generative biological language reasoning model. The generated protein result can include one or more predicted proteins described by an associated amino acid sequence and predicted protein structure, among other predicted properties.

In some embodiments, a biological programming specification is received that identifies at least one protein design condition in accordance with a biological programming language. For example, the received specification may include structural or functional condition requirements such as a defined secondary structure (e.g., an alpha helix between residues 15-30), a binding interface for a target molecule, or a symmetry constraint for the desired protein. Other conditions can include conditions related to properties such as stability, developability, and/or immunogenicity, particularly in the context of protein therapeutics. In various embodiments, the biological programming language allows the protein design conditions to be expressed in a modular and composable manner using high-level language, enabling the user to define complex architectures through human-readable syntax and/or through a graphical user interface. In some embodiments, the biological programming specification further specifies the target biological reasoning models used in generating the desired protein design. For example, one or more generative models can be targeted by the biological programming specification.

In some embodiments, a machine learning model is used to convert the biological programming specification to a model input format version for a biological reasoning model. For example, the machine learning model may be a large language model (LLM) trained to understand the structure and semantics of the biological programming language and the input requirements of target biological reasoning models, and to translate the specified constraints into conditioning input, such as conditioning tracks or embedding vectors, for the target model. Depending on the target model, the converted input format may include per-residue structural annotations, span-based motif tags, function tags, and/or global functional labels, among other conditions. In some embodiments, the model input format version is used as a conditioning input for the biological reasoning model to generate a protein design having the at least one protein design condition. For example, the conditioning input is used by the target model to predict a biological design that satisfies the specified protein design conditions defined by the biological programming specification. The conditioning input is encoded using the input format required by the model to produce the desired biological candidate protein designs.

In some embodiments, the biological programming specification is generated and/or refined via a natural language interface such as through a natural language interface of a large language model (LLM). For instance, a user can have a back-and-forth conversation with an LLM to generate and refine the biological programming specification. The user can specify requests for a design in a natural language format, such as by voice and using a chat interface, and the requests are converted to the appropriate biological programming language conditions within the biological programming specification. In various embodiments, the conversion is performed by a natural language processing agent, such as an artificial intelligence (AI) agent, including by an LLM-based agent. For example, by interfacing with an LLM, the user can utilize the LLM to generate a corresponding biological programming specification that can be compiled to conditioning input for a generative biological model. In some embodiments, the biological programming specification is generated using an AI-enhanced development environment, such as a programming environment enhanced with an AI agent for generating and refining the biological programming specification and its specified conditions, such as the requirements and constraints specified in the biological programming specification. For example, an LLM-based AI agent can be utilized to help generate the biological programming specification, including to validate the accuracy, performance, and feasibility of the specification.

The design and generation of biological objects, such as proteins, remains one of the most complex and impactful challenges in modem biotechnology. Traditional approaches rely on atomic-level manipulations or sequence-by-sequence specification, which are time-consuming, unintuitive, and often infeasible for non-experts. These traditional methods lack scalability and provide limited expressiveness for capturing high-level design goals including functional, structural, stability, developability, and immunogenicity goals. As a result, traditional approaches inhibit rapid prototyping, optimization, and particularly innovation in protein engineering and synthetic biology.

To address these limitations, the present disclosure introduces a novel biological programming framework that allows users to specify high-level design constraints through a modular, expressive programming language. These constraints can include function, functional motifs, primary, secondary, and tertiary structures, symmetry, binding interfaces, surface, stability, developability, and immunogenicity properties, among other design goals. In various embodiments, these constraints, specified using a biological programming language, are compiled into intermediate representations, which are then transformed into conditioning inputs suitable for various classes of generative biological models, including different large protein language models and model architectures. Example generative biological model architectures can include diffusion-based architectures and the disclosed multi-track biological language reasoning model.

A particularly novel aspect of the disclosed system is the use of large language models (LLMs) as compilers. The disclosed LLM-based compilers ingest the programming language specification and autonomously generate valid, realizable conditioning tracks and/or model inputs, optionally incorporating fine-tuning and reinforcement learning to improve biological results. The application of LLM-based compilers can decouple the user's intent from the complexities of model interfacing and dramatically improves usability, correctness, and design throughput. Additionally, the LLM-based compilers allow for the rapid integration of new generative biological models including models that support different constraints and/or are built using different conceptual frameworks and/or architectures. For example, the disclosed LLM-based compilers can rapidly integrate support for a new biological language reasoning model including support for translating the compiler's intermediate representation to an input format used by the new model.

Unlike prior systems which rely on hardcoded logic or fixed templates, the described platform enables dynamic and adaptive compilation strategies, supports hierarchical constraint structures, and can operate in both code- and UI-driven contexts. Furthermore, the model-agnostic biological programming language and compiler architecture allows the same input language to be reused across different biological language reasoning backend models. This enables seamless evolution as more advanced generative models are developed, ensuring long-term utility and broad applicability. The benefits of this approach are substantial: users can design biologically plausible proteins more efficiently and with greater precision, while maintaining flexibility to integrate future models and optimization strategies. This represents a significant technical advancement over prior systems and lays the foundation for scalable, programmable biological design including a design process that incorporates synthesis of experimental biological design candidates in a wet laboratory.

In connection with the disclosed biological programming language techniques and platform, a biological language reasoning model and corresponding service platform and architecture are disclosed. As described herein, the disclosed biological language reasoning techniques and system are able to process biological queries to generate and predict biological reasoning results. For example, a biological protein language model can process protein queries to generate and predict protein sequences, structure, and/or function, among other properties of a protein. Although primarily discussed with respect to proteins, the biological language reasoning techniques are applicable to other biological language domains as well. In some embodiments, the disclosed techniques are integrated into a biological reasoning service and the capabilities of the biological language model are exposed to clients. For example, a biological reasoning service incorporating a generative and predictive biological language reasoning model can reason over protein sequence, structure, and functions simultaneously. In some embodiments, a protein query can include masked portions of a protein sequence and/or structure and the output from the biological protein language model is the unmasked protein sequence and structure. As another example, a protein query can include different masked combinations of sequence, structure, and/or function descriptions in addition to other protein properties such as secondary structure and solvent accessible surface area. When the protein query is provided to the biological reasoning service, the results are the unmasked protein properties such as a protein's predicted sequence, structure, and/or functions. In various embodiments, the disclosed biological language reasoning model captures a complex biological understanding of the specific biological domain. For example, a protein language reasoning model can capture protein sequence, structure, secondary, tertiary, and quaternary structure, and/or functions at an atomic and/or amino acid level. In various embodiments, the disclosed biological language reasoning model is a multi-track model that allows the model to respond to queries along one or more different tracks. For example, a biological protein language reasoning model can be queried to predict and/or generate a protein's amino acid sequence, structure, secondary structure, protein solvent accessible surface area, and function.

In various embodiments, the disclosed biological language reasoning model is a multi-track model that utilizes multi-directional transformers. The utilized transformers are not restricted in a specific or single direction when considering biological context, such as for a protein sequence or structure. For example, the disclosed biological language reasoning model accepts masked input at any amino acid or residue position and for multiple amino acids or residue positions with respect to a query protein. Moreover, masking can apply to one or more tracks, such as sequence, structure, and/or function tracks, among other protein tracks. In particular, the disclosed biological language reasoning model can utilize tokens for each track. For example, tokenized protein structure can be utilized for structure conditioning and/or reasoning. In some embodiments, a protein's structure is tokenized into a set of structure tokens which are understood by the protein language model. Furthermore, within the biological language model and/or tokenizer, one or more self-attention blocks that incorporate geometric reasoning are utilized. A geometric reasoning module and each instance of a disclosed geometric reasoning block can process and be used in the encoding of structure including local and/or global structure such as across local protein structure and/or across the entire protein structure. For example, for protein structure, a geometric reasoning module can encode the structure of local amino acids based on their relative distance and direction to neighboring local amino acids. In some embodiments, the neighboring amino acids are determined by physical distance allowing the geometric reasoning module to encode complex physical structure properties of proteins at a local level. Using direction and distance factors between local neighboring amino acids, self-attention scores can be determined. In some embodiments, when determining self-attention scores, the determined direction properties are attenuated based on the determined distance properties. By using the disclosed structure tokenization techniques, a protein structure can be tokenized to significantly increase the efficiency and performance of protein generation and prediction. For example, a masked protein structure can be provided as an input query to generate a corresponding unmasked protein and its unmasked structure and sequence. The generated protein sequence can be used for a variety of applications including motif scaffolding, binder design, and antibody generation, among other applications. For example, a protein can be predicted and generated by the biological language model that conforms to a desired sequence and/or pattern, including a desired partial amino acid sequence and/or a desired partial protein structure such as a particular three-dimensional shape for a portion of the protein.

In some embodiments, an amino acid sequence of a protein is tokenized into amino acid sequence tokens. For example, a query protein is presented using at least a partial sequence of the query protein and where missing amino acids may be masked. The provided protein sequence is then tokenized into amino acid sequence tokens. In some embodiments, a structure of the protein is tokenized into structure tokens. Similar to the provided protein sequence, the query protein is presented using at least a partial structure of the query protein and where missing amino acids may be masked. The provided protein structure is then tokenized into structure tokens. In some embodiments, at least a portion of the amino acid sequence tokens and at least a portion of the structure tokens are combined into a combined training sequence data set having an amino acid sequence track and a structure track, wherein at least a portion of the structure track of the combined training sequence data set is masked. For example, using a multi-track approach with at least an amino acid sequence track and a structure track, the encoded sequence and structure tokens are combined into a combined training sequence data set. On various passes, different portions including different token portions may be masked and at varying or variable mask rates. In some embodiments, a language machine learning model is trained using the combined training sequence data set to predict one or more identities of the masked structure track portion of the combined training sequence data set. For example, a biological protein language machine learning model is trained using combined training sequence data sets with portions that are masked. By training with combined training sequence data sets with one or more masked portions, the overall robustness and prediction capabilities of a biological protein language machine learning model are significantly improved. For example, the trained model can recognize proteins including by predicting one or more identities of masked portions of a query protein. Although described with respect to a model with sequence and structure tracks, additional tracks are applicable as well. For example, a biological protein language machine learning model can be trained to predict any combination of protein properties such as protein sequence, structure, secondary structure, tertiary structure, quaternary structure, functions, and solvent accessible surface areas, among other properties. In some embodiments, the predicted properties utilize a token format and are predicted as predicted property tokens, such as predicted sequence, structure, secondary structure, function, and/or solvent accessible surface areas tokens. For example, by specifying a function that a query protein should exhibit, the corresponding function tokens can be provided to a biological protein language machine learning model as an input sequence that is combined with other specified property tokens. The combined input sequence data set can be used by the biological protein language machine learning model to predict corresponding sequence tokens to determine the amino acid sequence of the protein that exhibits the function specified.

In various embodiments, a biological protein language machine learning model utilizes protein structure tokens to efficiently encode protein structure. The protein structure can be encoded using a geometric reasoning process that includes determining geometric attention scores. In some embodiments, each amino acid in a query protein is tokenized. For example, each amino acid or residue in a protein can be tokenized in parallel to generate a set of structure tokens for a query protein including for a query protein with portions that are masked. In some embodiments, for a specific amino acid in a protein, physically neighboring amino acids of the specific amino acid are determined based on physical distances with respect to the specific amino acid in a local physical protein structure. For example, based on the local structure of a specific amino acid, the closest neighboring amino acids by distance are determined. By determining the closest neighboring amino acids based on distances with respect to structure rather than by their relative positions in the amino acid sequence, a significantly more accurate representation of structure is utilized. For example, by incorporating physical distances, the determined neighbors for a specific amino acid can include amino acids that are physically close but could appear relatively far apart when examined only by their relative positions in the protein's amino acid sequence. In various embodiments, the determination of the physically neighboring amino acids accounts for the three-dimensional structure of the protein such as when different portions or ends of a protein fold onto themselves. In particular, the use of physical distances to determine neighboring amino acids accounts for amino acids that are physically close in three-dimensional space despite being separated by many intervening amino acids in the protein's amino acid sequence. In some embodiments, the physical distance values are determined based on a local reference frame of the specific amino acid. In various embodiments, the K closest neighboring amino acids are determined by distance and the number of closest K neighbors can be configurable. In some embodiments, representations of the determined physically neighboring amino acids are included in a structure encoder input for the specific amino acid. For example, the determined local representation of an amino acid including references to its local neighboring amino acids is used as input for a structure encoder used to generate structure tokens. In some embodiments, the structure encoder input is provided to an autoencoder trained using geometric loss to determine a token representing the local physical protein structure for the specific amino acid. For example, the encoder of the autoencoder can generate a latent representation of a protein's local structure and that encoded representation can be further quantized, for example, with a codebook, to generate a structure token associated with an amino acid's structure within the protein.

In various embodiments, a geometric reasoning module is used to encode biological structure such as local protein structure into structure tokens. For example, a geometric reasoning module can utilize one or more geometric attention or geometric self-attention blocks. In some embodiments, a sequence state including representations of neighboring amino acids included in a local physical structure for a specific amino acid is received, wherein the neighboring amino acids at least include a first neighboring amino acid and a second neighboring amino acid. For example, a sequence state that includes a specific amino acid and references to its nearest neighboring amino acids is generated. The sequence state can be used to encode the local structure of the amino acid including its local structure with respect to its neighboring amino acids. In various embodiments, using the sequence state, attention scores can be determined by a geometric attention block. The geometric attention block can consider direction and/or distance between amino acids when determining an attention score.

In some embodiments, a direction query vector and a direction key vector are determined including by applying a first directional rotation transformation to at least a portion of a representation of the first neighboring amino acid included in the representations and applying a second directional rotation transformation to at least a portion of a representation of the second neighboring amino acid included in the representations. For example, a pair of neighboring amino acids are transformed into the same reference coordinate system. In some embodiments, the directional rotation transformation applied is based on each amino acid's local coordinate system. In some embodiments, a direction attention result is determined including by evaluating elements of the direction query vector and the direction key vector. For example, the direction query vector and the direction key vector can be multiplied to determine a direction attention result. In some embodiments, a dot product operation is performed on the corresponding direction query vector and direction key vector element. In some embodiments, at least the direction attention result is used to update the sequence state for an attention mechanism of a machine learning model. For example, a direction attention result can be used by the attention mechanism to calculate a geometric attention result using a value vector. The geometric attention result can further utilize other factors such as a distance attention result. In some embodiments, additional operations are performed to determine the geometric attention result, such as determining a value vector, applying a rotation transformation to the determined value vector, applying a softmax or normalization function to a direction attention result or a weighted direction attention result, and transforming a result back to the local reference frame, for example, by applying an inverse rotation transformation, to determine the resulting geometric attention result.

In some embodiments, the determined direction attention result is modified by a distance attention result. For example, the direction attention result can be modulated or attenuated based on distance such as determining a greater geometric attention value when neighboring proteins are closer. In various embodiments, the resulting final attention score can be a weighted sum of the direction and distance attention scores. In some embodiments, a distance query vector and a distance key vector are determined including by applying a first distance rotation transformation and a first distance translation transformation to at least the portion of the representation of the first neighboring amino acid included in the representations and applying a second distance rotation transformation and a second distance translation transformation to at least the portion of the representation of the second neighboring amino acid included in the representations. To ensure the applied transformations are consistent, in various embodiments, the different transformations for the first and second neighboring amino acids are consistent. For example, in various embodiments, the rotation matrices of the first distance rotation transformation and the first direction rotation transformation are the same, and the rotation matrices of the second distance rotation transformation and the second direction rotation transformation are the same. Furthermore, the application of different distance rotation and translation transformations allows the distance between the two neighboring amino acids to be determined by using the same frame of reference. In some embodiments, a distance attention result is determined including by evaluating elements of the distance query vector and the distance key vector. For example, a Euclidean norm operation can be performed with the corresponding distance query vector and the distance key vector elements to determine a distance attention result. In some embodiments, the operation performed corresponds to a Euclidean norm function on the difference between query and key vector values.

In some embodiments, at least the direction attention result and the distance attention result are used to update the sequence state for an attention mechanism of a machine learning model. For example, a weighted attention result based on the direction attention result and the distance attention result can be determined. In some embodiments, the weighted attention result is determined by subtracting a weighted distance term from a weighted direction term. For example, distance and direction term weights can be learned and applied for each attention head to determine a weighted attention result. Further, a softmax or normalization function can be applied to the weighted attention result and the result multiplied by a determined value vector that has been rotated using the appropriate rotation matrices. A transformation is applied to determine the resulting attention score, for example, by applying an inverse rotation transformation to transform the result back to the local frame of reference. In various embodiments, the geometric attention result is used to update the sequence state.

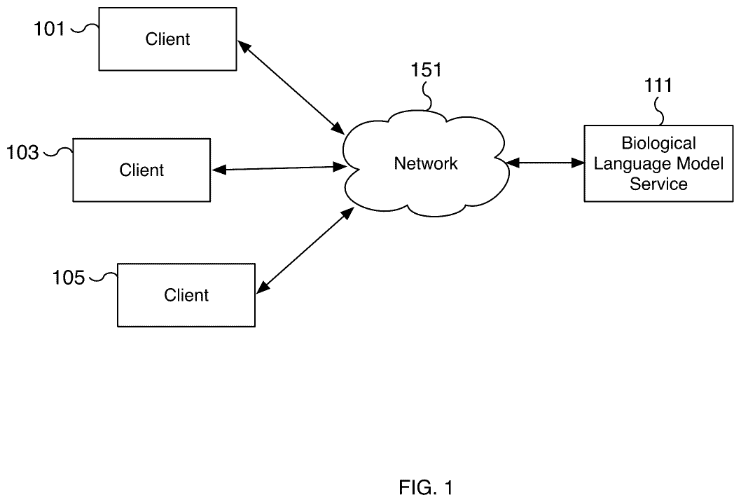

FIG. 1 is a block diagram illustrating an embodiment of a biological language reasoning platform that includes the ability to predict and generate biological language results using a biological language model. In the example shown, clients 101, 103, and 105 are network clients configured to access a biological language model hosted by biological language model service 111. Clients 101, 103, and 105 are communicatively connected to biological language model service 111 via network 151. Network 151 can be a public or private network. In some embodiments, network 151 is a public network such as the Internet. Biological language model service 111 provides biological language reasoning services including a service to predict and generate biological results such as a target protein's sequence, structure, and/or functions, among other properties of a target protein. For example, using biological language model service 111 via a client such as one of clients 101, 103, or 105, a user can provide a search query for a desired protein based on a partial target protein sequence and/or structure. A protein is then generated and predicted by biological language model service 111 that matches the provided protein constraints. In some embodiments, the predicted biological results can be visualized via a graphical user interface and further synthesized, such as via a wet lab. For example, a visual graphical user interface can be provided by biological language model service 111 to visually and interactively generate a search query and to subsequently visually inspect the resulting generated biological result.

In some embodiments, clients 101, 103, and 105 are each a network client device for interfacing with biological language reasoning services hosted by biological language model service 111. For example, each of clients 101, 103, and 105 can be configured with a network software client such as a browser to access biological language reasoning services of biological language model service 111 including the ability to predict and generate biological language results such as protein search results. A biological language reasoning query can be provided by clients 101, 103, and/or 105 in various appropriate formats such as a search query, a generative language prompt, a programming language, and/or a written or visual constraint description format, etc. In various embodiments, the clients 101, 103, and 105 are further utilized to manage and interface with biological language reasoning results, such as to review, iterate on, refine, and/or modify provided search results including provided predicted and generated protein results.

In some embodiments, biological language model service 111 is a cloud service that offers functionality for performing biological language reasoning including for predicting and generating biological language results. For example, biological language model service 111 can host a biological language model such as a multi-track biological protein language model that can predict both protein sequence and protein structure based on provided protein constraints, such as a partial protein sequence and/or protein structure. In some embodiments, biological language model service 111 can predict results when provided with multiple proteins, such as a sequence of proteins. For example, biological language model service 111 can predict the structure of the two or more query proteins including how they will fold and be held together. In various embodiments, the biological language model of biological language model service 111 is trained to capture the complex biological understanding of the targeted biological domain and is conditioned on and can be queried at the atomic level. For example, for protein generation and prediction, biological language model service 111 is trained based at least on local protein structure and captures the orientation of local amino acids and their physical relationship, including distance and direction, to neighboring amino acids.

In various embodiments, a multi-track biological language model of biological language model service 111 is based on a transformer model and includes multiple transformer blocks including transformer blocks with geometric attention and geometric reasoning. Further, the model and associated tokenizers are trained with one or more specialized geometric loss functions, for example, to improve the encoding of biological structure. In some embodiments, one or more specialized geometric loss functions can be used to compute physical structural differences between neighboring atoms and/or amino acids. For example, a geometric loss function can be used to determine direction loss and a separate geometric loss function can be used to determine distance loss.

In some embodiments, in addition to protein sequence and structure tracks, a multi-track biological protein language model of biological language model service 111 includes additional tracks such as protein function, protein feature, and additional protein structure tracks. For example, a multi-track biological protein language model can include secondary, tertiary, and/or quaternary protein structure tracks and protein feature tracks for defining constraints such as solvent accessible surface area. In various embodiments, the multi-track biological language model can be trained using biological structure tokens for improved efficiency, resource utilization, and performance.

In various embodiments, biological language model service 111 may further include various user interfaces for receiving biological language reasoning queries including biological language reasoning searches. For example, in some embodiments, biological language model service 111 provides a programming language interface for describing a search query such as a protein search query. The search query can provide context for the search including constraints for the targeted results. In some embodiments, the provided user interface includes a visual component for providing constraints such as sequence, structure, and/or function constraints.

In some embodiments, biological language model service 111 is interconnected with one or more web lab services, for example, for synthesizing predicted biological results. For example, biological language model service 111 may be further integrated with a web lab such as a third-party web lab for synthesizing a predicted biological result such as a predicted protein. The integrated third-party web lab can be provided with the predicted protein sequence for synthesizing the protein, for example, by assembling the predicted amino acids of the protein.

In various embodiments, the biological language reasoning services provided by biological language model service 111 are configured as a secure environment. For example, the provided biological language reasoning computing environment can be configured to adhere to security requirements including confidentiality and integrity requirements. The provided secure environment is particularly essential when multiple parties have different interests and security requirements. For example, the implemented requirements can be imposed by the biological language reasoning service provider, clients of the biological language reasoning services, and/or be attached to and/or associated with data used by the biological language reasoning service. Other parties and their respective security interests can exist as well, and their requirements can be reflected in the implemented security model. For example, the confidentiality and integrity of data such as training data provided by different clients such as clients 101, 103, and/or 105 can be protected including by isolating access to the provided data. Different clients can utilize their respective provided additional private data such as confidential private training data for improving and/or customizing a biological language model such as a foundational model hosted by biological language model service 111. The provided data can be secured, for example, using private and public key technologies and the encryption and/or decryption of the data can be managed by a key management service of biological language model service 111. For example, data including data in encrypted form, can be provided to and received at biological language model service 111 via a secure connection. In various embodiments, the encrypted data provided to biological language model service 111 is decrypted only under certain conditions such as only within a secure enclave and with the proper authorization. For example, encrypted data provided by clients can be decrypted only within a client's isolated secure enclave of biological language model service 111. The operations associated with and the environment of a client's secure enclave can be configured to meet a required security model, such as requirements for isolated compute, memory, and/or storage. Additional requirements include requirements on access to data, access to compute and other hardware resources, access to trained models including fine-tuned models, and/or access to training pipelines, among other requirements.

In some embodiments, biological language model service 111 offers a secure environment for processing client data and hosting client customized biological language reasoning models. For example, biological language model service 111 can offer key management services for use in transferring encrypted data to a secure enclave and the associated secure enclave for processing the transferred data and hosting trained models. Client data including confidential and/or sensitive data can be deployed to the secure enclave and only decrypted within the secure enclave. A biological language model can then be trained using the decrypted data. For example, a client can provide a specific dataset of confidential data for fine-tuning a foundational biological reasoning model and/or custom settings for a model including custom and/or confidential hyperparameters. For example, the fine-tuning of a trained foundational model using techniques such as Layered Regularization with Adversarial Projection (LORA) fine-tuning can be performed securely via biological language model service 111. A fully trained model can further be deployed within the enclave for performing inference including inference in response to biological language reasoning queries. For example, a fine-tuned model can be securely accessed via a client's secure enclave hosted by biological language model service 111. In some embodiments, a client's data and trained results, such as LORA weights, are securely stored in an account separate from an account used to securely store the foundational base model. An escrow provider provides a corresponding platform, such as via biological language model service 111, for allowing the fine-tuning and model interface to be performed across the two accounts. In various embodiments, the secure enclave allows client data including fine-tuned models trained with the data to be isolated from other environments including other client environments. The secure enclave further provides a secure and isolated compute environment, for example, for performing training and/or inference tasks. In various embodiments, a client's secure enclave is configured to meet a specific security model. For example, the operating environment of the secure enclave can be configured to not contain and/or not utilize persistent storage. Other examples of configuration/deployment settings include network connectively restrictions, interactive access restrictions, remote access restrictions, access restrictions based on client and/or host profiles, access requirements such as requiring multi-factor authentication, redaction requirements, and/or dedicated and/or isolated hardware requirements including dedicated compute and memory, among other configuration/deployment settings.

Although single instances of some components have been shown to simplify the diagram of FIG. 1, additional instances of any of the components shown in FIG. 1 may exist. For example, biological language model service 111 may include one or more cloud servers such as one or more machine learning training, machine learning inference, and/or web application servers and one or more databases utilized by the cloud servers. Additionally, clients 101, 103, and 105 are example client devices for accessing and utilizing the services of biological language model service 111. Although three clients are shown (clients 101, 103, and 105), many more additional clients can exist and access the services of biological language model service 111. In some embodiments, components not shown in FIG. 1 may also exist.

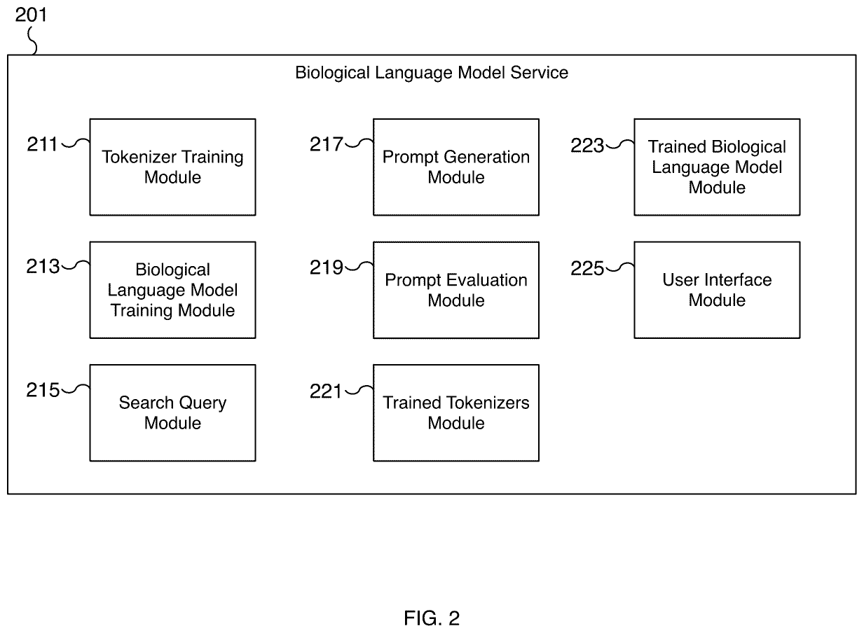

FIG. 2 is a block diagram illustrating an embodiment of a biological language model service for generating and predicting biological language reasoning results. In the example shown, biological language model service 201 is a cloud-based service for applying a biological language model to perform biological language reasoning. In various embodiments, the biological language model can be applicable to different biological domains such as for protein prediction and generation. Biological language model service 201 includes tokenizer training module 211, biological language model training module 213, search query module 215, prompt generation module 217, prompt evaluation module 219, trained tokenizers module 221, trained biological language model module 223, and user interface module 225. In some embodiments, biological language model service 201 is biological language model service 111 of FIG. 1. In some embodiments, the clients accessing and utilizing the services of biological language model service 201 include clients 101, 103, and/or 105 of FIG. 1.

In some embodiments, biological language model service 201 includes multiple processing modules for performing biological language reasoning. In various embodiments, one or more of the modules shown may not exist and/or additional modules may exist. In some embodiments, the functionality of one or more of the modules may be merged into a single module or split out across multiple different modules. In some embodiments, biological language model service 201 is implemented by one or more cloud servers and one or more data stores such as one or more databases including distributed databases. In some embodiments, cloud servers of biological language model service 201 can include machine learning training and inference servers as well as web application servers.

In some embodiments, tokenizer training module 211 is a processing module for training tokenizers used by biological language model service 201. For example, tokenizer training module 211 can be used to train a variety of tokenizers based on the tracks available for a multi-track biological language model of biological language model service 201. Example tokenizers can include a tokenizer for protein sequence, protein structure, and protein feature, among other protein tracks, as applicable. In some embodiments, tokenizer training module 211 is used to train a protein structure tokenizer that encodes protein structure into tokens based on the local structure of amino acids relative to neighboring amino acids. In various embodiments, the tokenized format for biological structure allows a biological language model to predict and generate biological results more efficiently and with greater emphasis resource utilization. Moreover, the trained tokenizers may be autoencoders and can include a decoding module for decoding tokens. For example, a protein structure decoder can decode protein structure tokens into a protein structure. Similarly, a protein sequence decoder can decode protein sequence tokens into a protein sequence.

In some embodiments, biological language model training module 213 is a processing module for training a multi-track biological language model of a biological language model service. For example, biological language model training module 213 can be used to train a multi-track biological protein language model to predict and generate protein language results based on masked input. In some embodiments, the different tracks can include protein sequence and protein structure tracks. Additional tracks can include secondary, tertiary, and/or quaternary protein structure tracks, protein feature tracks for defining constraints such as solvent accessible surface area, and a protein function track, among others. In various embodiments, a multi-track biological language model can be trained using biological structure tokens for improved efficiency, resource utilization, and performance, among other technological benefits. For example, protein structure can be pre-tokenized and used to train a multi-track biological protein language model using protein structure tokens. In various embodiments, the multi-track biological language model utilizes multiple multi-directional transformers and includes processing modules for geometric attention and reasoning.

In some embodiments, biological language model training module 213 includes functionality for training a multi-track biological language model using both experimental and synthetic training data. For example, experimental biological data including experimental protein sequence and structure data can be processed into training data. Similarly, synthetic biological data including predicted protein sequence and structure data generated using machine learning techniques can be processed into training data. The experimental and synthetic training data can be scored based on their respective accuracy to reflect their experimental and/or synthetic nature. By utilizing both experimental and synthetic training data, the trained model is conditioned with a greater understanding of the targeted biological language. In particular domains, such as with respect to protein structure, experimental may be scarce and the use of scored synthetic data allows a biological protein language model to develop a more thorough understanding of protein language.

In some embodiments, search query module 215 is a processing module for receiving and preparing a biological language reasoning query. Search query module 215 can support different query formats including formats based on a generative language prompt, a search query programming language, written constraint descriptions, and visual constraint descriptions, among others. In various embodiments, search query module 215 can receive and process a search query thereby preparing the query for prompt generation and subsequent prompt evaluation. For example, based on a received search query, search query module 215 can generate multiple derivative inference passes for a multi-track biological language model to narrow a search space for optimal prediction results. In some embodiments, search query module 215 along with user interface module 225 provide an interface for clients (such as clients 101, 103, and/or 105 of FIG. 1) to interface with biological language model service 201. For example, utilizing search query module 215, clients can perform searches for structure prediction, protein design, motif scaffolding, binder design, and/or antibody generation, among other biological language reasoning applications.

In some embodiments, prompt generation module 217 is a processing module for generating a generative artificial intelligence (AI) prompt for use with a biological language reasoning model of trained biological language model module 223 such as a biological protein language model. In some embodiments, the generative AI prompt is created by compiling and/or parsing a biological reasoning programming language. In some embodiments, the generative AI prompt is created by prompt evaluation module 219 using at least in part a prompt template customized for the biological language reasoning model and/or tokenizing the appropriate input using trained tokenizers module 221. For example, prompt generation module 217 can provide the appropriate context and specifics, such as tokenized sequence, structure, and/or function, for the various tracks that are applicable for a multi-track biological language reasoning model. In some embodiments, prompt generation module 217 interfaces with search query module 215 to create one or more generative AI prompts to solve a biological reasoning search query. For example, prompt generation module 217 can generate a sequence of iterative prompts including prompts based on past inference results to narrow the field of search when addressing a biological reasoning search query. In some embodiments, prompt generation module 217 can perform additional preprocessing for prompt data when generating a generative AI prompt. For example, structure data such as local amino acid structure information can be converted by prompt generation module 217 into the appropriate structure format usable by a biological language reasoning model.

In some embodiments, prompt evaluation module 219 is a processing module for evaluating a biological language generative artificial intelligence (AI) prompt using a trained biological language reasoning model of trained biological language model module 223. In some embodiments, the generative AI prompt is created by prompt generation module 217 and addresses the different tracks of a multi-track biological language reasoning model. For example, a generative AI prompt for a trained multi-track biological protein language model can be used to predict protein sequence, structure, and/or function, depending on the configured tracks of the selected model and the selectively generated masked input. In various embodiments, prompt evaluation module 219 initiates the evaluation of the generative AI prompt using the appropriate trained biological language model to generate and predict a biological language result. For example, prompt evaluation module 219 can evaluate a generative AI prompt to predict a protein sequence, structure, and/or function using a trained biological protein language model. In various embodiments, prompt evaluation module 219 interfaces with search query module 215 and/or user interface module 225 to provide biological language reasoning model inference results to a user in response to evaluating a prompt using the selected trained biological language model.

In some embodiments, trained tokenizers module 221 is a module for interfacing with trained tokenizers for use with a trained biological language model. For example, trained tokenizers module 221 includes access to multiple trained tokenizers for tokening input for different tracks of a multi-track biological language model. In some embodiments, the provided tokenizers can include a protein sequence tokenizer, a protein structure tokenizer, and/or a protein function tokenizer, among other tokenizers. In various embodiments, trained tokenizers module 221 interfaces with search query module 215, prompt generation module 217, and/or prompt evaluation module 219 to provide token results. In various embodiments, the tokenizers are trained using tokenizer training module 211.

In some embodiments, trained biological language model module 223 is a module for interfacing with a trained biological language model such as a trained biological protein language model. In various embodiments, trained biological language model module 223 can provide inference results when provided with a biological language prompt. In some embodiments, trained biological language model module 223 may utilize additional training and/or finetuning modules for improved prediction results. For example, one or more additional models in addition to a foundation biological language model can be utilized as part of an inference pipeline of trained biological language model module 223. Moreover, in various embodiments, trained biological language model module 223 can select between multiple models depending on context including based on factors such as biological domain, resource availability, configuration, accessibility, and/or cost, among other factors. For example, trained biological language model module 223 may provide different models trained for different conditions and the appropriate model is selected. In various embodiments, trained biological language model module 223 interfaces with search query module 215, prompt generation module 217, and/or prompt evaluation module 219 to provide biological language reasoning inference results. In some embodiments, trained biological language model module 223 includes access to third-party models such as a third-party structure prediction model. In various embodiments, the models of trained biological language model module 223 are trained using biological language model training module 213.

In some embodiments, user interface module 225 is a processing module for providing a user interface for interfacing with biological language model service 201. For example, user interface module 225 can provide visual, textual, and/or graphical user interfaces, among other forms of user interfaces, for exposing and utilizing the services of biological language model service 201. In some embodiments, the provided user interface is a programmatic, command-line, graphical, dialog-based, augmented reality-based, and/or virtual reality-based user interface. For example, user interface module 225 can allow users to create and execute biological language reasoning queries as well as to review, iterate on, refine, and/or modify the provided biological language reasoning results. In some embodiments, a provided graphical user interface allows a user to define a query protein and to view the predicted and generated protein in response to the protein query. In some embodiments, the interface is a programming language interface, and the user provides a programming language description of the desired query. The generated results can be further processed including by using a biological language programming language to process the received generated results as return data. In some embodiments, user interface module 225 provides an application programming interface (API) to expose the services of biological language model service 201. For example, a provided biological language model service API can allow for the automated execution of search queries by biological language model service 201.

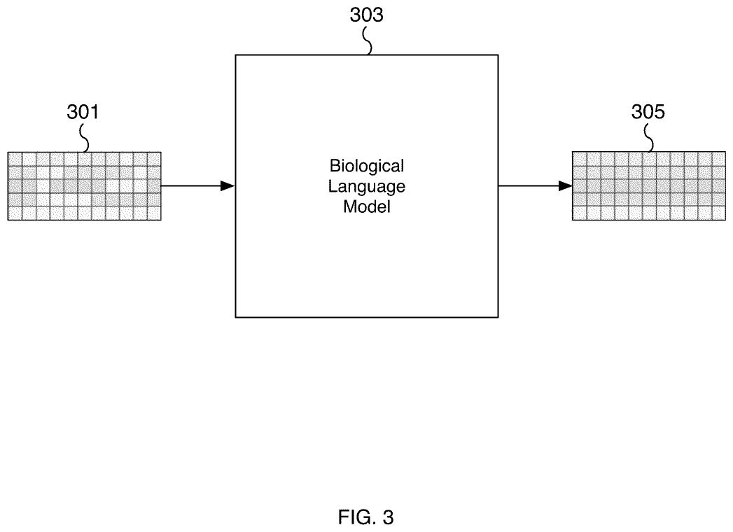

FIG. 3 is a block diagram illustrating an embodiment of a multi-track biological language reasoning model. In the example shown, biological language model 303 is a biological language model that receives masked multi-track input 301 and predicts multi-track output 305. In some embodiments, masked multi-track input 301 is created in response to a search query such as a search query processed by search query module 215 of FIG. 2 and converted into a generative artificial intelligence (AI) biological language prompt for biological language model 303 by prompt generation module 217 of FIG. 2. In some embodiments, the control flow for evaluating a biological language prompt by biological language model 303 is performed by prompt evaluation module 219 of FIG. 2. In some embodiments, biological language model 303 is a trained biological language model trained by biological language model training module 213 of FIG. 2 and managed by trained biological language model module 223 of FIG. 2. In some embodiments, biological language model 303 utilizes one or more tokenizers that are trained by tokenizer training module 211 of FIG. 2 and managed by trained tokenizers module 221 of FIG. 2. Although masked multi-track input 301, biological language model 303, and multi-track output 305 are described in the context of a biological protein language reasoning, other biological language domains are applicable as well based on the disclosed architecture, techniques, and platform discussed herein.

In some embodiments, masked multi-track input 301 includes masked input for each of the multiple tracks supported by biological language model 303. In the example shown, biological language model 303 can correspond to a protein language model and masked multi-track input 301 includes five protein related tracks, each with masked elements. For example, the five tracks for a protein language model can correspond to input for an amino acid sequence, primary structure, secondary structure, solvent accessible surface area, and function. In various embodiments, the input for each track is tokenized input with corresponding tokens corresponding to each amino acid of the protein for each track of masked multi-track input 301. A specification associated with a desired property of a protein, such as its sequence, structure, secondary structure, solvent accessible surface area, and function, can be converted into at one or more input tokens. For example, for the amino acid sequence of a protein, the amino acid sequence input track corresponds to amino acid sequence tokens for each unmasked amino acid sequence of the query protein, and for the primary structure of a protein, the primary structure input track corresponds to learned structure tokens for each unmasked amino acid structure of the query protein. In various embodiments, the amino acid sequence tokens can be determined by a mapping, encoding, machine learning, and/or another appropriate approach. For example, the set of known and/or supported amino acids can be mapped to a set of amino acid tokens. In some embodiments, an amino acid token vocabulary can be learned for the amino acid sequence input track. Similarly, for the secondary structure of the query protein, the secondary structure input track corresponds to secondary structure tokens for each unmasked secondary structure for each amino acid of the query protein, for function properties of the query protein, the feature input track corresponds to function tokens for unmasked features for each amino acid of the query protein, and for the solvent accessible surface area of the query protein, the solvent accessible surface area input track corresponds to solvent accessible surface area tokens for unmasked solvent accessible surface area for each amino acid of the query protein. In various embodiments, the different input tracks can apply a mapping, encoding, machine learning, and/or another appropriate approach to generate corresponding tokens. For example, a vocabulary of secondary structure elements can be determined and used to map secondary structure to secondary structure tokens. In some embodiments, a dictionary of secondary structure of proteins (DSSP) algorithm, a structural identification (STRIDE) algorithm, and/or another approach is used to generate the vocabulary of secondary structure elements for mapping to secondary structure tokens. In some embodiments, for solvent accessible surface area, a solvent accessible surface area metric can be tokenized by bucketing the solvent accessible surface area metric. For example, a float value corresponding to a depth in a protein can be binned to generate a solvent accessible surface area token. In the example shown, the boxes of masked multi-track input 301 that are blank correspond to masked input whose values will be predicted as output values in multi-track output 305 by biological language model 303. In various embodiments, when the different tracks are taken together, masked multi-track input 301 corresponds to combined input tokens that are a combined input sequence data that specify at least a portion of a protein. The combined input sequence can be provided to a biological protein language machine learning model to predict one or more missing tokens.

In some embodiments, multi-track output 305 includes the unmasked values for each amino acid's properties for each track supported by biological language model 303. For example, multi-track output 305 can include the unmasked amino acids of a corresponding masked query protein's sequence with respect to masked multi-track input 301. As another example, multi-track output 305 can include a predicted structure for masked amino acids of the structure track for a query protein with respect to masked multi-track input 301. As yet another example, multi-track output 305 can include predicted functions for masked amino acids of the function track for a query protein with respect to masked multi-track input 301. In various embodiments, biological language model 303 similarly predicts the values of corresponding masked properties for other tracks of biological language model 303 such as secondary structure and solvent accessible surface area tracks. In the example shown, multi-track output 305 shows no masked values since each corresponding masked value of masked multi-track input 301 now has a corresponding unmasked value predicted by biological language model 303. In various embodiments, the values of multi-track output 305 can be tokenized values. For example, a decoder of a trained auto-encoder tokenizer can be used to decode the predicted token value into a more accessible output result. As one particular example, a protein sequence decoder can decode predicted sequence tokens into a more convenient, accessible, and manageable protein sequence format usable for protein synthesis. Similarly, a protein structure decoder can decode predicted structure tokens into a more convenient and manageable protein structure format including one that can then be used to visualize the predicted protein structure. In some embodiments, a property token for an amino acid, such as a function token, can correspond to one or more properties such as one or more functions of the amino acid. For example, a single function token can be decoded to the set of all functions predicted for a particular amino acid of the query protein.

In some embodiments, biological language model 303 is a multi-track deep learning model that utilizes multi-directional transformers. The utilized transformers are not restricted in a specific or single direction when considering biological context, such as for a protein sequence or structure. For example, when trained for protein language reasoning, biological language model 303 can accept, via masked multi-track input 301, masked input at any amino acid or residue position for a query protein. Moreover, masking can apply to any of the tracks supported by biological language model 303, such as sequence, structure, and/or function tracks, among other protein tracks. In various embodiments, biological language model 303 can utilize tokens for each track. For example, a tokenized protein structure can be utilized for structure conditioning and/or reasoning. In some embodiments, biological language model 303 can further utilize one or more self-attention blocks that can incorporate geometric reasoning. A geometric reasoning module and each instance of a disclosed geometric reasoning block can process and be used in the encoding of a local structure. In various embodiments, in response to the provided masked multi-track input 301, a protein language reasoning version of biological language model 303 predicts the masked values of masked multi-track input 301 to generate a target protein described by multi-track output 305 in response to the query protein constrained by the unmasked values of masked multi-track input 301.

In some embodiments, although not shown in FIG. 3, biological language model 303 may receive additional forms of input other than the tokenized input tracks of masked multi-track input 301. For example, biological language model 303 may receive additional input in the form of partial structure input instead of a tokenized version of the structure. In some embodiments, a query protein structure is provided in a user-accessible format that allows users to more easily interface with biological language model 303 including by providing structure constraints in an unencoded format for the query protein. For example, local structure for amino acids of a query protein can be described using three-dimensional coordinates associated with relevant atoms. When configured to receive non-tokenized input, biological language model 303 can be structured to include one or more geometric reasoning blocks that allow the model to express an internal understanding of protein structure including local protein structure and/or structure across the entire protein. In some embodiments, although non-tokenized input is provided to the biological language model 303, the input can be tokenized as a pre-processing step such as a pre-processing step for preparing masked multi-track input 301.

In some embodiments, biological language model 303 includes one or more conditioning tracks and/or conditioning tensors. For example, a biological language program can be compiled to generate conditioning input corresponding to requirements and/or constraints for biological language model 303 to utilize during inference. In various embodiments, biological language model 303 is trained to support a biological language program specification and can include one or more conditioning tracks that can be conditioned using a biological language program based on the biological language program specification.

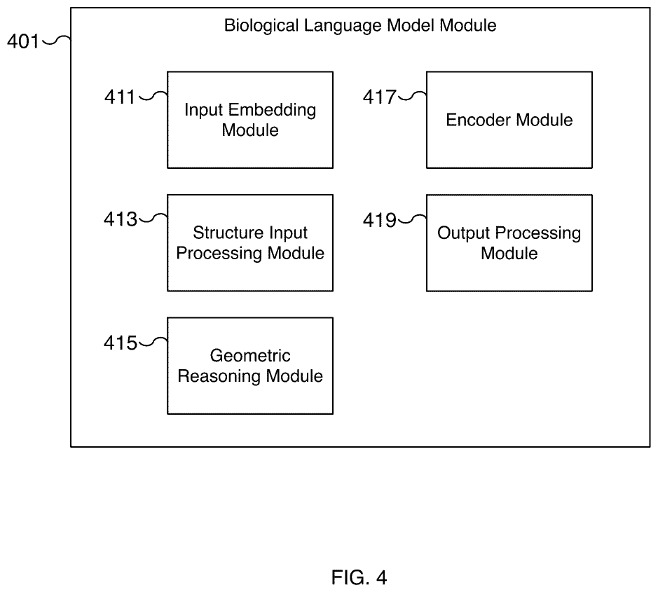

FIG. 4 is a block diagram illustrating an embodiment of a biological language model module capable of generating and predicting biological language reasoning results. In various embodiments, biological language model module 401 corresponds to components and architecture aspects for using a deep learning model to perform biological language reasoning. In the example shown, biological language model module 401 includes input embedding module 411, structure input processing module 413, geometric reasoning module 415, encoder module 417, and output processing module 419. In various embodiments, biological language model module 401 corresponds to trained biological language model module 223 of FIG. 2 and biological language model 303 of FIG. 3. For example, biological language model module 401 corresponds to at least a portion of the functionality used to train biological language model 303 of FIG. 3 and to perform inference using biological language model 303 of FIG. 3.

In some embodiments, input embedding module 411 is a processing module for embedding input provided to the model such as provided input tokens. For example, the different input tokens for the different tracks of a multi-track biological language model are embedded into embedding vectors using an embedding layer of input embedding module 411. In some embodiments, a position-wise sum operation is performed by input embedding module 411. For example, for biological protein language reasoning, the position-wise sum operation can be performed relative to each amino acid of the query protein. In various embodiments, corresponding embedding vectors based on the length L of a query protein and an embedding dimension are generated by input embedding module 411 and capture the provided semantic meaning for each of the L amino acids of the query protein.

In some embodiments, structure input processing module 413 is a processing module for processing user provided structure data. For example, structure information can be provided to a multi-track biological language model in a non-tokenized format, such as a user-accessible format that allows users to interface with a biological language model more easily. Rather than requiring biological structure be presented in a tokenized format, users can provide structure constraints in an unencoded format, including standardized structure formats. For example, for a query protein, the local structure of amino acids can be provided in a standardized and/or documented structure format. In some embodiments, the received structure input utilizes backbone frames for each amino acid including specifying relevant atoms of an amino acid based on their three-dimensional spatial coordinates. In some embodiments, the received structure input utilizes a different set of atoms for each amino acid including, for example, a set that uses all atomic coordinates for an amino acid. For non-proteins, the structure input can include coordinates for any number of relevant atoms including all atoms of at each specific position. In various embodiments, structure input processing module 413 receives the provided structure input and performs any necessary pre-processing before feeding the processed structure data to the biological language model. For example, a biological language model can be configured to receive non-tokenized structure data for conditioning on local structure. In some embodiments, the structure data is received at a transformer block configured with geometric attention.

In some embodiments, geometric reasoning module 415 is a processing module for performing geometric reasoning on structure input data. For example, geometric reasoning module 415 can be utilized to encode local structure information based on provided local structure data. In some embodiments, a transformer block of encoder module 417 can utilize geometric reasoning module 415 to perform geometric attention on provided structure data including structure data that has not been tokenized to encode local structure context. In various embodiments, geometric reasoning module 415 can include one or more of layer normalization, self-attention, geometric attention, and feed-forward blocks. For example, layer normalization can be performed prior to self-attention, geometric attention, and feed-forward processing. In some embodiments, the geometric reasoning process performed by geometric reasoning module 415 is similar to the geometric reasoning performed when tokenizing structure data.

In some embodiments, encoder module 417 is a processing module for processing the embedded semantic input generated by input embedding module 411. For example, encoder module 417 can apply attention mechanisms to the embedded input to allow the model to consider the full context of the query target such as a query protein. In various embodiments, encoder module 417 includes multiple transformer blocks including self-attention and geometric attention blocks. For example, encoder module 417 can include multiple layers of transformers, each layer applying attention mechanisms to consider, for example, the context of different amino acids of a query protein. In some embodiments, encoder module 417 includes multi-directional transformers and can unmask properties of the query object, such as a query protein, at different positions such as at different amino acid locations. At the completion of processing for encoder module 417, the output can be a set of intermediate or hidden representations for each position of the query object. In some embodiments, the outputted representations correspond to a set of output values or logits that require additional processing to generate a biological query output result.

In some embodiments, output processing module 419 is a processing module for applying an output layer to generate biological language model results. In some embodiments, output processing module 419 projects the logits corresponding to intermediate or hidden outputted representations determined by encoder module 417 for each track of the multi-track model. For example, output processing module 419 can determine sequence, structure, and function values for corresponding sequence, structure, and function tracks of a multi-track model. In various embodiments, output processing module 419 determines the multi-track output values in the format of output tokens. The output tokens can then be decoded using a decoder module of the corresponding tokenizer.

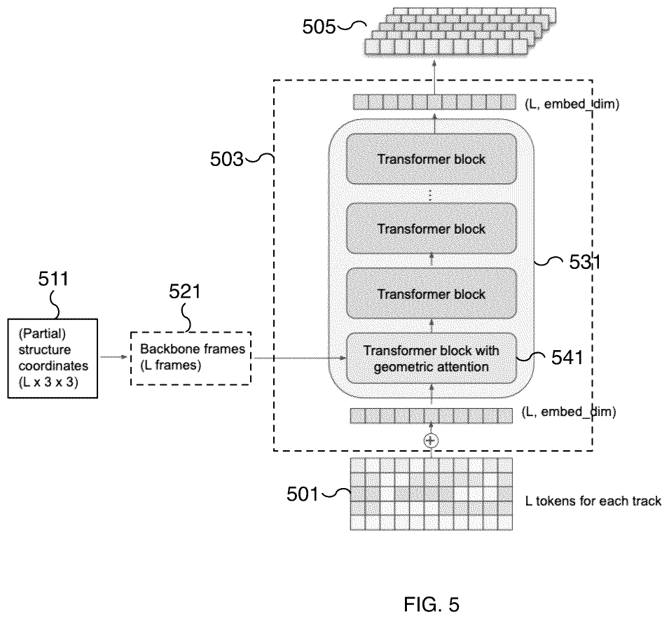

FIG. 5 is a block diagram illustrating an embodiment of a multi-track biological protein language model module capable of generating and predicting biological protein language reasoning results including structure results. In the example shown, masked multi-track input 501 is provided as input for a specific query protein to multi-track biological protein language model 503. Also shown in FIG. 5 is structure input data 511 that corresponds to the local amino acid structure data for the query protein. The query protein is described by L amino acids, each with three backbone atoms with each backbone atom described by three coordinates corresponding to X, Y, and Z coordinate values. Structure input data 511 is converted to backbone frames 521. Both masked multi-track input 501 and backbone frames 521 are received at multi-track biological protein language model 503 and used to predict query protein results corresponding to multi-track output 505. In various embodiments, backbone frames 521 is used to augment constraints specified in masked multi-track input 501.

In some embodiments, multi-track biological protein language model 503 is trained by biological language model training module 213 of FIG. 2 and the trained model is then managed by trained biological language model module 223 of FIG. 2. In some embodiments, inference with multi-track biological protein language model 503 is initiated by prompt evaluation module 219 of FIG. 2. In some embodiments, multi-track biological protein language model 503 is further implemented via biological language model module 401 of FIG. 4 and backbone frames 521 is converted by biological language model module 401 of FIG. 4. For example, structure input data 511 can be converted to backbone frames 521 by structure input processing module 413 of FIG. 4. In some embodiments, masked multi-track input 501 is masked multi-track input 301 of FIG. 3, multi-track biological protein language model 503 is biological language model 303 of FIG. 3, and/or multi-track output 505 is multi-track output 305 of FIG. 3.

As shown in FIG. 5, multi-track biological protein language model 503 includes encoder block 531 with multiple transformer blocks including transformer block with geometric attention 541 that receives backbone frames 521. In various embodiments, each of the transformer blocks of encoder block 531 can implement an attention mechanism such as a self-attention or geometric attention mechanism. In some embodiments, the embodiment of encoder block 531 is implemented by encoder module 417 of FIG. 4 and the corresponding geometric attention mechanism of the displayed transformer is implemented by geometric reasoning module 415 of FIG. 4.

In various embodiments, a position-wise sum operation is performed on masked multi-track input 501 by multi-track biological protein language model 503. For example, the L input tokens per track of masked multi-track input 501 are embedded into vectors by an embedding layer such as one implemented by input embedding module 411 of FIG. 4. Also shown in FIG. 5 is the output of encoder block 531 and its chain of transformers. In various embodiments, this output corresponds to intermediate or hidden representations for each position of the query protein. In some embodiments, the determined representations correspond to a set of output values or logits. Multi-track biological protein language model 503 projects the determined representations (or logits) for each track of multi-track output 505, unmasking any corresponding masked input values of masked multi-track input 501. In some embodiments, the output layer processing is performed by output processing module 419 of FIG. 4.

In some embodiments, structure input data 511 utilizes a coordinates format for describing biological structure such as protein structure. In the example of FIG. 5, the structure data of structure input data 511 can include the coordinates for each of the three backbone atoms of each of the L amino acids of a protein. The backbone atoms can correspond to nitrogen (N), alpha-carbon (CA), and carbon (C) atoms and each can be described by a set of three-dimensional spatial coordinates corresponding to X, Y, and Z coordinates. Structure input data 511 can include fewer than L amino acids when only a partial structure is defined. For a protein with L amino acids, the backbone coordinates have size L×3×3. In some embodiments, backbone frames 521 utilizes a frame format by determining a backbone frame for each protein amino acid backbone. For example, a frame can include the coordinates of the alpha-carbon atom and a 3×3 rotation matrix for the frame defined by the nitrogen, alpha-carbon, and carbon atoms. In some embodiments, the alpha-carbon is placed at the origin and the nitrogen atom defines the X-axis. Although specific spatial and structure formats are described, alternative formats are appropriate as well. For example, another reference point other than the nitrogen atom of a backbone can be used to define the X-axis. Moreover, although FIG. 5 is shown with backbone frames 521, other frame formats other than ones based on a backbone frame are appropriate as well. In some embodiments, structure input data 511 and multi-track biological protein language model 503 are alternatively configured to utilize a structure format that relies on a different set of atoms (other than backbone atoms) such as a format that utilizes all atomic coordinates.

In various embodiments, the block diagram of FIG. 5 corresponds to only a partial view of a multi-track biological language model. For example, some of the components shown of multi-track biological protein language model 503 may be simplified and certain dimensions flattened for ease of demonstration. Additionally, certain components of FIG. 5 may be replicated to maximize performance such as by performing additional processing in parallel. In order to emphasize certain features of multi-track biological protein language model 503, some components may exist that are not shown, such as certain layers and functional components of the multi-track biological protein language model.

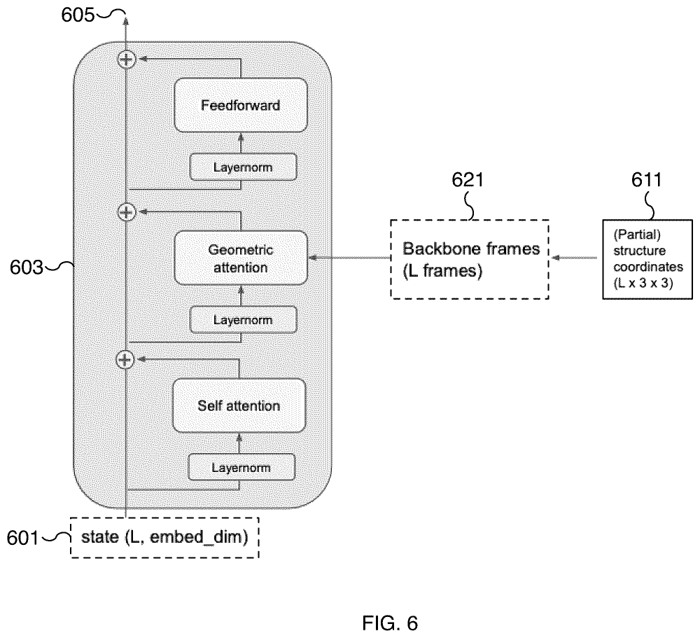

FIG. 6 is a block diagram illustrating an embodiment of a transformer block with a geometric attention mechanism of a multi-track biological language model. In the example shown, state information 601 is provided by an upstream component of a multi-track biological language model to transformer block with a geometric attention 603. Transformer block with a geometric attention mechanism 603 includes multiple functional components including layer normalization, geometric attention, and feed forward blocks. In some embodiments, the multi-track biological language model is multi-track biological protein language model 503 of FIG. 5, transformer block with a geometric attention 603 is transformer block with geometric attention 541 of FIG. 5, structure input data 611 is structure input data 511 of FIG. 5, and/or backbone frames 621 is backbone frames 521 of FIG. 5.

As shown in FIG. 6, structure input data 611 corresponding to local amino acid structure data for the query protein is converted to backbone frames 621. Backbone frames 621 is received at the geometric attention block of transformer block with a geometric attention 603 for applying geometric attention based on the provided local structure described by structure input data 611. The arrangement of functional components including layer normalization, geometric attention, and feed forward blocks of transformer block with a geometric attention 603 determines an updated representation of the state 601 and is outputted as updated state 605. Updated state 605 can be provided to a downstream stage of the multi-track biological language model such as a downstream transformer block. In various embodiments, updated state 605 reflects the application of self-attention, geometric attention, and feed forward networks. As shown in FIG. 6, the provided local structure information can be limited to a partial description of the query protein. Although FIG. 6 is shown with backbone frames 621, other frame formats other than ones based on a backbone frame are appropriate as well. In some embodiments, structure input data 611 and transformer block with a geometric attention 603 are alternatively configured to utilize a structure format that relies on a different set of atoms (other than backbone atoms) such as a format that utilizes all atomic coordinates.

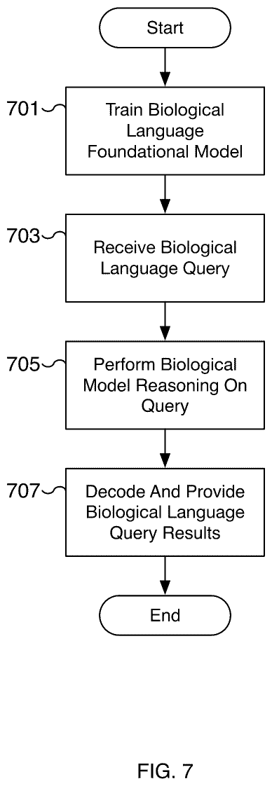

FIG. 7 is a flow chart illustrating an embodiment of a process for performing biological language reasoning using a biological language reasoning model. For example, using the process of FIG. 7, a biological language query can be answered using a trained biological language reasoning model, such as a biological language reasoning model trained for generating and predicting proteins. In various embodiments, the model is a multi-track model that generates and predicts biological properties of a target object, such as a query protein. When applied to proteins, the model can predict both sequence and structure. Additional protein properties can include secondary structure, tertiary structure, quaternary structure, and/or functions at an atomic and/or amino acid level. In various embodiments, the disclosed biological language reasoning model is a multi-track model that allows the model to respond to queries along one or more different tracks. For example, a biological protein language reasoning model can be queried to predict and/or generate a protein's amino acid sequence, structure, secondary structure, protein solvent accessible surface area, and function. In some embodiments, the process of FIG. 7 is performed by a biological language model service such as biological language model service 111 of FIG. 1 and/or biological language model service 201 of FIG. 2. In some embodiments, the biological language model is biological language model 303 of FIG. 3.

At 701, a biological language foundational model is trained. For example, a biological language foundation model can be trained on billions or more parameters and with training data containing multiple tracks. For particular biological domains, such as for protein generation and prediction, the amount of experimental data may be limited. To address this limitation, a biological language foundation model can be trained on both experimental and synthetic data. For example, data sources can be scored based on their origin, accuracy, and/or quality when training and corresponding quality metrics can be specified during inference. In various embodiments, the trained biological language foundation model is a multi-track transformer encoder model and utilizes transformers that are not restricted in a specific or single direction when considering biological context, such as for a protein sequence or structure. For example, a biological protein language reasoning model is trained to predict protein sequence, structure, and/or function, among other protein properties even when provided with partial or masked query protein data. Additional tracks for a protein language model include secondary structure, tertiary structure, quaternary structure, function, and solvent accessible surface area tracks.

In some embodiments, the biological language foundational model is further trained on biological interactions including molecular interactions within a biological object and/or between two or more biological objects such as multiple proteins. For example, multimer data such as data based on proteins binding together is used to train the model to predict molecular binding between two or more proteins. In various embodiments, the protein binding data included in the training data is sourced from peptides, protein-protein binders, antibodies, and/or nanobodies, among other sources. Furthermore, data for interactions within a biological object such as long-range tertiary contacts for single proteins can also be used for training molecular interaction predictions. In some embodiments, one or more specialized tokens such as chain break and/or epitope tokens are used for describing multiple objects and/or molecular interaction locations. Moreover, the training for biological interactions can be performed as a pretraining and/or finetuning step. For example, the biological language foundational model can be finetuned using techniques such as supervised finetuning and direct preference optimization (DPO) techniques to emphasize and align the prediction results to binding tasks.

In various embodiments, the biological language foundation model is trained using a range of mask rates. By training on variable mask rates and without a specific masking objective, the trained biological language foundation model can predict multiple tokens for each forward pass. For example, a trained biological protein language foundation model can predict a complete protein structure (and sequence) when queried with a partial protein sequence. Similarly, a trained biological protein language foundation model can predict a complete protein sequence when provided with a partial protein structure. In various embodiments, the model is trained for promptability characteristics that allow the model to predict across and within different tracks such as to predict a sequence from function, function from sequence, structure from function, structure from partial structure, etc.

In some embodiments, the training of the biological language foundation model includes training one or more tokenizers. For example, each track of the multi-track biological language foundation model can utilize a tokenizer, such as a protein sequence tokenizer, protein structure tokenizer, and a protein function tokenizer, etc. In various embodiments, one or more of the tokenizers are trained to encode, decode, and understand specialized tokens such as chain break and epitope tokens. For example, a chain break token can be inserted between two biological objects such as between two proteins. As an input token, the chain break token signals to the biological language reasoning model that multiple objects are described in the input, such as a binding target protein followed by a masked binder protein. Based on the provided input, the biological language reasoning model predicts the structural binding result of the two biological objects including by generating the identities for masked positions of the binder protein. In some embodiments, a chain break token as an output token in the prediction output results indicates where one object ends and another starts. For example, a chain break token in the output prediction results can be used to indicate the end of a binding target protein sequence and the start of a binder protein sequence. As another example, a tokenizer can be trained to learn epitope tokens that are used to indicate an epitope of a binding target protein. For example, epitope tokens can be inserted in the binding target protein sequence at the start and end of an epitope location. In some embodiments, a different start epitope token and end epitope token is used. In various embodiments, epitope tokens are used to train the biological language reasoning model to understand the positions/locations of a binding target protein to which a generated binder protein should bind. In some embodiments, an epitope token is a property and/or subset of a function token. In some embodiments, alternatives to an epitope token are used, such as a binder track for the model or a conditioning epitope tensor. In various embodiments, in the context of proteins, a protein structure tokenizer is trained to encode local structure including local amino acid structure for proteins. For improved performance and resource utilization, a biological language foundation model can then be trained on pre-tokenized data such as pre-tokenized structure data. The use of pre-tokenized data for training results in significant performance and resource utilization advantages.

In some embodiments, the training step performed at 701 includes additional optional training steps such as fine-tuning, post-training, and/or model alignment. For example, one or more additional post-training steps can be performed to improve the model's performance, robustness, interoperability, safety, and/or accuracy, among other desired goals, as well as to customize and/or adapt the model for specific tasks. In some embodiments, the additional training is performed on a new dataset, for example, that is prepared for a specific targeted task.

At 703, a biological language query is received. For example, a biological reasoning query is received for the biological language foundation model trained at 701. The query can be a search query for a specific biological object such as a target protein that meets the provided constraints. In some embodiments, the query can be for a combination of biological objects such as for a masked binder protein that binds to an unmasked binding target protein. For example, constraints can be provided based on unmasked values for the different tracks of the multi-track biological language foundation model trained at 701. In some embodiments, the query is processed and used to perform multiple inference passes of the biological language foundation model. For example, multiple inference passes can be performed to iteratively narrow and refine the search query results.

In various embodiments, the biological language query received at 703 can be provided in one or more supported formats. For example, the query can be a biological language foundation model prompt with input tokens. As another example, the query can utilize a biological language programming language. The biological language programming language can define the context of the query object such as a query protein and constraints and/or target goals for the query. In some embodiments, the query is generated and/or provided at least in part via a visual interface, such as one that allows a user to specify protein sequence, structure, function, etc. using a graphical user interface tool. Other protein properties the query can search can include solvent accessible surface area and secondary structure of a query protein.

In some embodiments, the received query can further specify an accuracy required for the corresponding query result. For example, the search query can specify confidence values for prediction results including specifying the type of training data the prediction result should rely on. In some embodiments, the query can be conditioned on properties of training data such as whether the training data is based on experimental and/or synthetic training data.

At 705, biological model reasoning is performed on the received biological language query. For example, the biological language search query received at 703 is solved using the biological language foundation model trained at 701. In some embodiments, one or more inference passes of the biological language foundation model are performed to refine the search query results. In some embodiments, the biological language model is used to unmask the masked parameters for each track of the multi-track biological language foundation model. For example, sequence, structure, and function, among other tracks are unmasked to generate and predict a biological result such as a target protein. In various embodiments, the biological model reasoning is performed by applying multiple transformer encoder layers including multiple attention mechanisms of the biological language foundation model. The attention mechanisms can include self-attention and/or geometric attention mechanisms based on the biological language learned during the training phase performed at 701. In some embodiments, additional post-processing such as fine-tuning is performed to refine the prediction results of the biological language foundation model.

At 707, the biological language query results are decoded and provided. For example, the inference results from the biological language foundation model are processed to determine search query results. In some embodiments, the inference results determined at 705 are tokenized output and the tokenized output is decoded at 707. For example, a predicted set of protein sequence tokens including protein sequence tokens for one, two, or more proteins can be decoded into one or more user-accessible protein amino acid sequences including protein sequences that can be used for protein synthesis. In some embodiments, the predicted set of protein sequence tokens includes a predicted binder protein bound to a binding target protein. The binder protein and binding target protein may be separated by a chain break token. As another example, a predicted set of protein structure tokens can be decoded into a user-accessible protein structure that can be visualized. In various embodiments, the biological language query results are provided in a requested format. For example, the results can be provided according to a programming language format that allows for the use of a biological programming language to process the search query results programmatically. As another example, the results can be presented in a visual and/or multi-media format. For example, the results can be provided to a client via a graphical user interface including virtual and/or augmented user interfaces. In some embodiments, the results are provided and allow for a user (or program) to review, iterate on, refine, and/or modify the biological language query results and corresponding search.

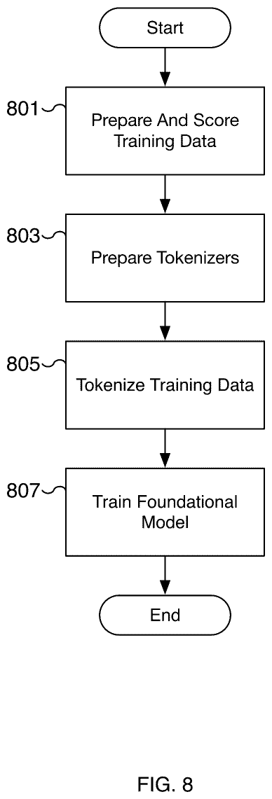

FIG. 8 is a flow chart illustrating an embodiment of a process for training a biological language reasoning model. For example, using the process of FIG. 8, a biological language reasoning model can be trained to perform biological language reasoning. In various embodiments, the biological language foundation model is trained as a multi-track transformer encoder model and utilizes transformers that are not restricted in a specific or single direction when considering biological context, such as for a protein sequence or structure. For example, a biological protein language reasoning model is trained to predict protein sequence, structure, and/or function, among other protein properties even when provided with partial or masked query protein data. Additional tracks for a protein language model include secondary structure, tertiary structure, quaternary structure, function, and solvent accessible surface area tracks. In some embodiments, the process of FIG. 8 is performed at 701 of FIG. 7 by a biological language model training module such as biological language model training module 213 of FIG. 2 of a biological language model service such as biological language model service 111 of FIG. 1 and/or biological language model service 201 of FIG. 2. In some embodiments, the biological language model trained using the process of FIG. 8 corresponds to the model associated with biological language model 303 of FIG. 3, biological language model module 401 of FIG. 4, and/or multi-track biological protein language model 503 of FIG. 5.

At 801, training data is prepared and scored. For example, training data for the target biological domain of the biological language reasoning model is prepared. For particular biological domains, such as for protein generation and prediction, the amount of experimental data may be limited. To address this limitation, a biological language foundation model can be trained on both experimental and synthetic data. For example, data sources can be scored based on their origin, accuracy, and/or quality when training and corresponding quality metrics can be specified during inference. Similarly, the training data can be scored with confidence values corresponding to the accuracy of the data. In various embodiments, the training data addresses all the tracks of the multi-track model. For example, for a multi-track protein language model that predicts sequence, structure, and function, training data can be prepared for each track using both experimental and/or synthetic data. For certain tracks, more experimental data may be available and for other tracks, a mixture of experimental and synthetic data may be utilized. In some embodiments, the training data is additionally masked for masked token prediction.

At 803, tokenizers for the biological language reasoning model are prepared. For example, for each track of the multi-track biological language reasoning model, a tokenizer can be prepared. In some embodiments, the tokenizer for a track is an autoencoder and/or utilizes an encoder-decoder architecture. For example, a protein structure tokenizer is prepared by training a protein structure encoder and decoder for tokenizing protein structure and decoding predicted structure tokens. Similarly, a function tokenizer can be trained based on an identified function vocabulary. In various embodiments, the tokenizers can be trained at the atomic level of the biological domain. For example, for protein generation and prediction, the tokenizers can be trained to encode tokens for each amino acid location of a query protein. In various embodiments, the training process for various tokenizers utilizes one or more loss functions. For example, a structure tokenizer can utilize one or more geometric loss functions to encode a semantic understanding of geometric structure. In some embodiments, tokenization is performed in real-time or near real-time rather than requiring a process that first creates a vocabulary. For example, by tokenizing in real or near real-time, the tokenizers can create new tokens as raw training data is sent to the model for training.

At 805, training data for the biological language reasoning model is tokenized. For example, the training data can be pre-tokenized prior to training. By tokenizing the training data and feeding pre-tokenized input to the biological language reasoning foundational model, aspects of the foundational model can be streamlined. Similarly, the tokenization of complex model input data, such as three-dimensional protein atomic structure data, prior to training significantly improves performance when training the biological language reasoning foundational model. For example, the resources and time required for training the biological language reasoning foundational model are significantly reduced when the model is at least partially trained with tokenized input rather than requiring the model to perform all tokenizing tasks at training. In various embodiments, the training data can be stored in a tokenized format for future and additional rounds of training.

At 807, a biological language reasoning foundational model is trained. For example, using the training data prepared at 801 and tokenized at 805, the biological language reasoning foundational model is trained. In some embodiments, the training is performed using different mask rates allowing the biological language reasoning foundational model to unmask biological features without direction restrictions and/or to predict multiple tokens for each forward pass. For example, the trained biological language foundation model can utilize transformers that are not restricted in a specific or single direction when considering biological context, such as for a protein sequence or structure. In various embodiments, the training process is an iterative process and the performance of the model is monitored using testing and validation data sets generated from the training data prepared at 801. In some embodiments, the training process utilizes one or more loss functions, such as loss functions based on token prediction losses.

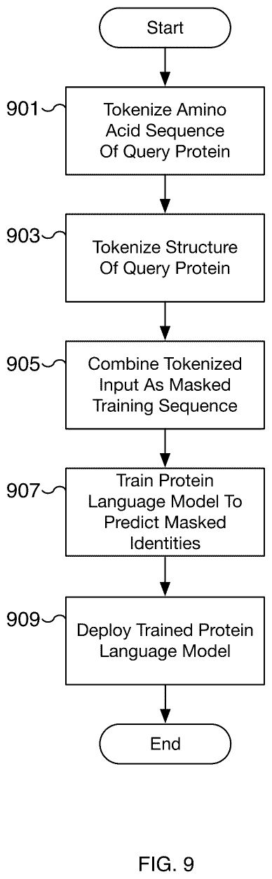

FIG. 9 is a flow chart illustrating an embodiment of a process for training a biological protein language model. For example, using the process of FIG. 9, a biological protein language model can be trained to perform biological protein language reasoning. In some embodiments, the protein language model is trained as a multi-track transformer encoder model and utilizes transformers that are not restricted in a specific or single direction when considering protein context, such as amino acid location for protein sequence, structure, or function. For example, the trained biological protein language model can predict at least a complete protein sequence and structure when provided with partial or masked input. As another example, the trained biological protein language model can predict a binder protein sequence and corresponding binder protein structure when provided with partial or full binding target protein and a masked binder protein as input. Additionally, the trained model can be extended to other protein properties such as function and secondary structure, among others. In some embodiments, the process of FIG. 9 is performed at 701 of FIG. 7 and/or at 805 and/or 807 of FIG. 8 by a biological language model training module such as biological language model training module 213 of FIG. 2 of a biological language model service such as biological language model service 111 of FIG. 1 and/or biological language model service 201 of FIG. 2. In some embodiments, the biological protein language model trained using the process of FIG. 9 corresponds to the model associated with biological language model 303 of FIG. 3, biological language model module 401 of FIG. 4, and/or multi-track biological protein language model 503 of FIG. 5.

At 901, the amino acid sequence of a query protein is tokenized. For example, each amino acid in a sequence of a query protein is converted to an input token. In various embodiments, the protein sequence tokenizer is trained based on the set of available amino acids to encode each amino acid of the protein sequence. Other approaches are appropriate as well. For example, the protein sequence tokenizer may also utilize a dictionary lookup to tokenize the amino acid sequence by mapping each amino acid in the amino acid sequence to an amino acid token. In some embodiments, each amino acid is associated with a unique identifier and the applied tokenizer can utilize a transformer model configured with attention mechanisms to tokenize the provided amino acid identifier. In some embodiments, the tokenization is performed in real-time or near real-time rather than requiring a process that first creates a vocabulary. For example, by tokenizing in real or near real-time, the tokenizers can adapt to new tokens as they appear.

At 903, the structure of the query protein is tokenized. For example, the structure of a query protein is received including a description of local amino acids. In some embodiments, the local amino acids of the protein are described by their spatial positioning including by three-dimensional coordinates of their backbone atoms. In various embodiments, the local structure is encoded using a trained protein structure tokenizer that applies geometric attention to embed local context into structure tokens. For example, each amino acid is tokenized in the context of its nearest neighboring amino acids. In some embodiments, the protein structure tokenizer is trained to encode the context of each amino acid of a protein with respect to the distance and direction of each amino acid to its determined nearest neighboring amino acids by physical distance. In some embodiments, the trained protein structure tokenizer is an autoencoder. The encoder can be applied to generate structure tokens and the corresponding decoder can be applied to decode protein structure tokens into a protein structure. In some embodiments, the structure tokens are discrete tokens created by quantizing an encoder output using a learned codebook.

At 905, the tokenized input is combined as a masked training sequence. For example, the tokens created for sequence and structure at 901 and 903, respectively, are combined as a training sequence. In various embodiments, the training sequence is masked at specific mask rates to allow the protein language model to learn a contextual understanding of protein sequence and structure. In various embodiments, the protein language model is a multi-track transformer model and the input tokens are combined according to sequence and structure tracks.

At 907, a protein language model is trained to predict masked identities. For example, using the training data created at 905 from masked training sequences, the protein language model is trained to predict masked identities. With a sufficient amount of masked training sequence data, including sequences masked at different mask rates, the model can learn a contextual understanding of protein sequence and structure. In various embodiments, the training process is an iterative process and the performance of the model is monitored using testing and validation data sets. In some embodiments, the training process utilizes one or more loss functions including geometric loss functions to encode a semantic understanding of geometric structure into the trained biological language reasoning foundational model.

At 909, the trained protein language model is deployed. For example, the trained weights of the model are deployed for use with a biological language model service. In some embodiments, the model is accessible via a biological language reasoning service that accepts search queries and provides model responses to the received queries. In some embodiments, additional training may be performed including fine-tuning steps, such as for specific search queries or query types, and/or a post processing pipeline may be configured. For example, a post-processing pipeline can be configured that is performed to convert model output to a format usable by client users.

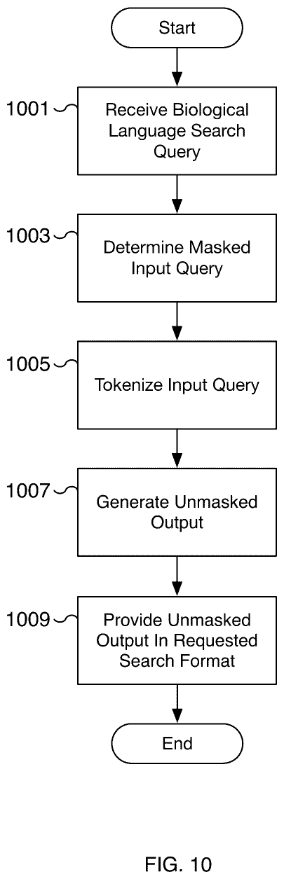

FIG. 10 is a flow chart illustrating an embodiment of a process for applying a biological language reasoning model to a biological language search query. For example, using the process of FIG. 10, a biological language search query is received, and a result is determined using a biological language reasoning model. In various embodiments, the search query is processed and corresponding input tokens for the model are created with selective portions of the input masked. In response to the masked input, the model can predict the identities of the masked portions. In some embodiments, the process of FIG. 10 is performed at 703, 705, and/or 707 of FIG. 7 by a biological language model service such as biological language model service 111 of FIG. 1 and/or biological language model service 201 of FIG. 2. In some embodiments, the search query can be processed and evaluated using a combination of the functional modules including search query module 215 of FIG. 2, prompt generation module 217 of FIG. 2, prompt evaluation module 219 of FIG. 2, and/or trained biological language model module 223 of FIG. 2. In some embodiments, the biological language reasoning model used for inference results corresponds to biological language model 303 of FIG. 3, biological language model module 401 of FIG. 4, and/or multi-track biological protein language model 503 of FIG. 5.

At 1001, a biological language query is received. For example, a user provides a biological language query with the expectation of a query result. In some embodiments, the query is provided using a programming language format such as using a biological language programming language that allows the query and results to be processed programmatically. In some embodiments, the biological language query corresponds to a generative artificial intelligence (AI) prompt that can be processed by the model. In some embodiments, the biological language query is received via an application programming interface (API). Other formats and interfaces for the biological language query can be appropriate as well including visual and/or graphical interfaces. For example, a biological language query can be constructed using a graphical user interface including by specifying structure constraints. In various embodiments, the search query can be processed to convert the query into a native format understood by the biological language model. For example, a search query constructed using a biological language programming language format can be compiled and/or parsed and converted into a native format understood by the biological language model.

At 1003, a masked input query is determined. For example, based on the biological language query received at 1001, the query is processed to determine a masked input query for the biological language reasoning model. In various embodiments, different tracks corresponding to different properties of the biological object are evaluated to determine which portions are masked and which are unmasked. For example, the provided constraints can be a partial description such as a partial physical structure with corresponding masked and unmasked identities. In some embodiments, the query is processed and a corresponding generative artificial intelligence (AI) prompt is created along with the masked input query.

At 1005, the input query is tokenized. For example, the masked input query determined at 1003 is tokenized to create input tokens for each track of the biological language reasoning model. In some embodiments, each track can utilize a different trained tokenizer such as a trained autoencoder. For example, a trained structure tokenizer can tokenize a structure track of the masked input query while applying geometric attention. In various embodiments, the input tokens can embed a learned context of the input query object and can include learned local context information. In some embodiments, the input query includes and/or references multiple biological objects such as a sequence of two or more proteins. During the tokenization performed at 1005, the individual biological objects can be delineated. For example, the tokenized representations of two proteins can be separated by a break token to delineate when one protein ends and another starts.

At 1007, an unmasked output is generated. For example, the biological language reasoning model is evaluated with the input tokens to determine an output result. The output result predicts and unmasks the masked identities of the masked input query determined at 1003. In some embodiments, the input tokens are evaluated by the biological language reasoning model based on a provided generative artificial intelligence (AI) prompt. For example, a provided generative AI prompt provides direction and context for evaluating the input tokens created at 1005. The biological language reasoning model can utilize multiple transformer encoder layers including multiple attention mechanisms of the biological language reasoning model when generating values including unmasked values for the query result. In various embodiments, the output including the unmasked output is provided in a tokenized format. For example, the predicted structure can be a set of structure tokens with predicted values for any previously masked values as defined by the search query.

At 1009, the unmasked output is provided in the requested search format. For example, the unmasked output results generated at 1007 using the biological language reasoning model are processed to provide search query results in a requested format. In some embodiments, the unmasked output is tokenized by the biological language reasoning model and a decoding step is performed to decode tokens into more accessible formats. For example, a predicted set of structure tokens can be decoded into a user-accessible structure that can be visualized. In some embodiments, the search format utilizes a format such as a biological language programming language and the unmasked output results generated at 1007 are converted into a programming language result. Other formats may be applicable as well. For example, the unmasked output can be packaged as an application programming interface (API) result to conform with a pre-defined API interface.

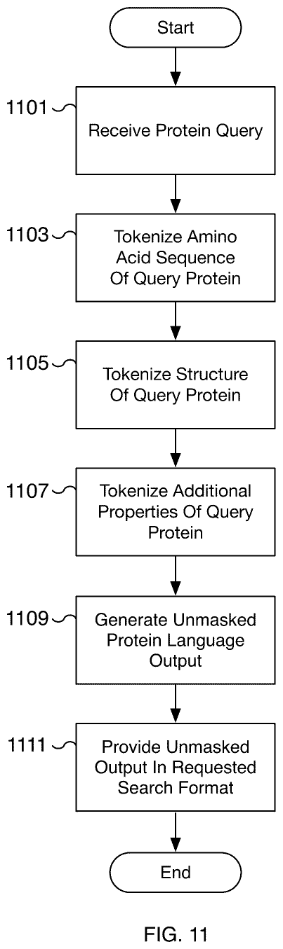

FIG. 11 is a flow chart illustrating an embodiment of a process for applying a biological protein language model to a biological protein language search query. For example, using the process of FIG. 11, a biological language reasoning model trained to understand and reason using a protein language on query proteins is applied to answer biological protein language search queries. In various embodiments, the biological language reasoning model is a multi-track biological protein language model with tracks at least for protein sequence and structure. Although described in FIG. 11 with protein sequence and structure tracks, additional tracks, such as secondary structure, function, and other protein property tracks can be supported as described herein. In various embodiments, a protein search query is processed and corresponding protein input tokens are created with selective portions of the input masked. In response to the masked input, the biological protein language model can predict the identities of the masked portions such as masked portions of the query protein's sequence and/or structure. In some embodiments, the process of FIG. 11 is performed at 703, 705, and/or 707 of FIG. 7 and/or using the process of FIG. 10 by a biological protein language model service such as biological language model service 111 of FIG. 1 and/or biological language model service 201 of FIG. 2. In some embodiments, the protein search query can be processed and evaluated using a combination of the functional modules including search query module 215 of FIG. 2, prompt generation module 217 of FIG. 2, prompt evaluation module 219 of FIG. 2, and/or trained biological language model module 223 of FIG. 2. In some embodiments, the biological protein language model used for inference results corresponds to biological language model 303 of FIG. 3, biological language model module 401 of FIG. 4, and/or multi-track biological protein language model 503 of FIG. 5.

At 1101, a biological protein language query is received. For example, a user provides a biological protein language query with the expectation of a protein query result. The biological protein language query can describe a specific query protein to predict a suitable protein (or proteins) that matches the constraints expressed by the biological protein language query. The biological protein language query may also describe a sequence of proteins, such as a combination of two or more proteins. For example, a query can include a binder protein and a binding target protein where the predicted binder protein should bind to the binding target protein. The biological protein language query may be constructed to predict one or more of the included proteins by unmasking any unknown properties including how the proteins of the query will fold and be held together. In some embodiments, the result of the biological protein language query is a set of proteins that resolve and/or satisfy the biological protein language query. In some embodiments, the biological protein language query is provided using a programming language format such as using a biological language programming language that allows the biological protein language query and results to be processed programmatically. In some embodiments, the biological protein language query corresponds to a generative artificial intelligence (AI) prompt that can be processed by the biological protein language model. In some embodiments, the biological protein language query is received via an application programming interface (API). Other formats and interfaces for the biological protein language query can be appropriate as well including visual and/or graphical interfaces. In various embodiments, the biological protein language query can be processed to convert the biological protein language query into a native format understood by the biological protein language model. For example, a biological protein language query constructed using biological language programming language format can be compiled and/or parsed and converted into a native format understood by the biological protein language model.

At 1103, the amino acid sequence of the biological protein language query protein is tokenized. For example, each amino acid in a sequence of the query protein is converted to an input token. In various embodiments, the protein sequence tokenizer is trained based on the set of available amino acids to encode each amino acid of the protein sequence. Other approaches are appropriate as well. For example, the protein sequence tokenizer may also utilize a dictionary lookup to tokenize the amino acid sequence by mapping each amino acid in the amino acid sequence to an amino acid token. In some embodiments, each amino acid is associated with a unique identifier and the applied tokenizer can utilize a transformer model configured with attention mechanisms to tokenize the provided amino acid identifier.

At 1105, the structure of the biological protein language query protein is tokenized. For example, the structure of the query protein is received including a description of local amino acids. In some embodiments, the local amino acids of the query protein are described by their spatial positioning including by three-dimensional coordinates of their backbone atoms. In various embodiments, the local structure is encoded using a trained protein structure tokenizer that applies geometric attention to embed local context into structure tokens. For example, each amino acid is tokenized in the context of its nearest neighboring amino acids. In some embodiments, the protein structure tokenizer is trained to encode the context of each amino acid of a protein with respect to the distance and direction of each amino acid to its determined nearest neighboring amino acids by physical distance. In some embodiments, the trained protein structure tokenizer is an autoencoder. The encoder can be applied to generate structure tokens and the corresponding decoder can be applied to decode protein structure tokens into a protein structure. In some embodiments, the structure tokens are discrete tokens created by quantizing an encoder output using a learned codebook.

At 1107, additional properties of the biological protein language query protein are tokenized. For example, input for additional tracks supported by the biological protein language model such as secondary structure, tertiary structure, quaternary structure, function, and solvent accessible surface area tracks are tokenized using the appropriate trained track tokenizer. In some embodiments, non-tokenized input may be received and processed by the model. For example, the biological protein language model can receive non-tokenized input, such as protein structure data, in addition to or as an alternative to tokenized input. In various embodiments, for non-tokenized input such as non-tokenized protein structure input, the biological protein language model can implement attention mechanisms such as geometric attention mechanisms to encode local geometric understanding.

In some embodiments, a function track supported by the biological protein language model allows each position of a biological object to be associated with and/or described by zero, one, or more functions. For example, for a protein object, an initial vocabulary of function labels is determined. The function labels can be sourced from one or more known public databases and/or one or more private data sources, among other sources. When applied to protein function, each position of a protein can be associated with any number of the known functions. For example, for a protein with sequence length L and a function vocabulary size K, there are L×K applicable labels. For a determined function vocabulary, vocabulary size K can exceed 30,000 entries corresponding to 30,000 or more different function annotation tags. In various embodiments, due to the large number of function annotation tags applicable for each protein sequence position, the function label input is compressed as part of tokenization. For example, at each sequence position, the function labels at that position can be converted into a single Term Frequency-Inverse Document Frequency (TF-IDF) vector. In various embodiments, each converted TF-IDF vector represents the saliency of text keywords that describe the applicable functions in plain text descriptions. In some embodiments, a TF-IDF vector is constructed using associated text function descriptions from relied upon data sources. Example descriptions include InterPro and Gene Ontology text descriptions. In some embodiments, the TF-IDF vectors are compressed and tokenized by converting the vectors into discrete integer tokens for input to the biological protein language model. For example, a set of locality sensitive hashes (LSHs) can be applied to each TF-IDF vector. In various embodiments, each LSH produces a single token within a set range, such as between 0 and 255. Multiple integer tokens can be generated for each protein position by using multiple LSHs. For example, when a fixed set of eight LSHs are applied, eight integer tokens can be generated at each position to represent the semantic information from the function text descriptions. In various embodiments, each LSH is computed using a fixed number of hyperplanes sampled from a normal distribution. The LSH is applied by determining which side of each hyperplane the TF-IDF vector falls via the sign of the inner product of the TF-IDF vector with the hyperplane's normal vector. When using eight hyperplanes, a list of eight hyperplane-side binary values can be converted into an integer format such as an integer between 0 and 255.

At 1109, an unmasked protein language output is generated. For example, the biological protein language reasoning model is evaluated with the input tokens determined at 1103, 1105, and 1107 to predict an output result. In various embodiments, the tokenized input may be combined into a multi-track input for the biological protein language model. The corresponding output of the biological protein language model may be a multi-track output that unmasks the masked identities associated with the tokenized query protein. In some embodiments, the input tokens are evaluated by the biological protein language reasoning model based on a provided generative artificial intelligence (AI) prompt. For example, a provided generative AI prompt provides direction and context for evaluating the input tokens created at 1103, 1105, and 1107. The biological protein language reasoning model can utilize multiple transformer encoder layers including multiple attention mechanisms of the biological protein language model when generating values including unmasked values for the protein query result. In various embodiments, the output including the unmasked output is provided in a tokenized format. For example, the predicted protein structure can be a set of protein amino acid structure tokens with predicted values for any previously masked values as defined by the search query.

At 1111, the unmasked output is provided in the requested search format. For example, the unmasked output results generated at 1109 using the biological protein language reasoning model are processed to provide search protein query results in a requested format. In some embodiments, the unmasked output is tokenized by the biological protein language reasoning model and a decoding step is performed to decode tokens into more accessible formats. For example, a predicted set of protein structure tokens can be decoded into a user-accessible structure that can be visualized. As another example, a predicted set of protein sequence tokens can be decoded into a protein sequence format that allows the predicted protein to be experimentally synthesized. In some embodiments, the protein query search format utilizes a format such as a biological language programming language and the unmasked output results generated at 1109 are converted into a programming language result. Other formats may be applicable as well. For example, the unmasked output can be packaged as an application programming interface (API) result to conform with a pre-defined API interface.

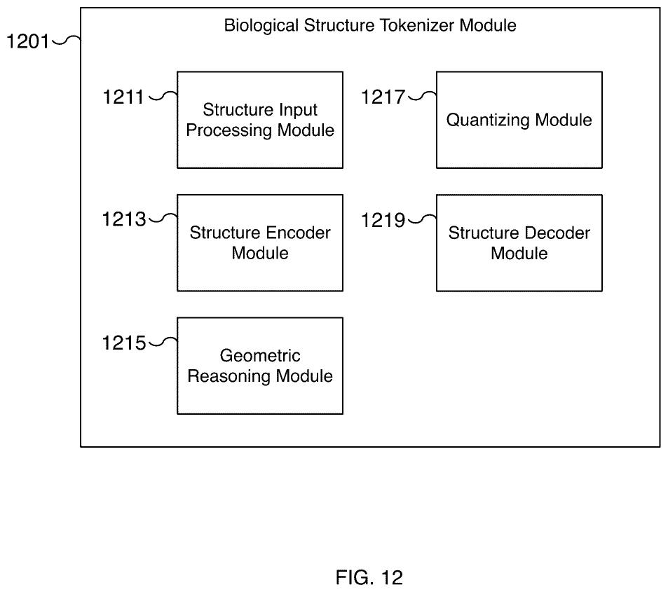

FIG. 12 is a block diagram illustrating an embodiment of a tokenizer for biological structure. For example, biological structure tokenizer module 1201 is trained to tokenize biological structure data for a specific biological domain such as for protein structure. Biological structure tokenizer module 1201 can be trained to tokenize structure data at the atomic level or at a coarser configured granularity depending on the application. Biological structure tokenizer module 1201 includes structure input processing module 1211, structure encoder module 1213, geometric reasoning module 1215, quantizing module 1217, and structure decoder module 1219. In some embodiments, biological structure tokenizer module 1201 is used to tokenize biological structure for training a biological language reasoning model, as a pre-processing step for performing inference using a biological language reasoning model, and/or for decoding structure token results generated by a trained biological language reasoning model. In some embodiments, biological structure tokenizer module 1201 is trained using tokenizer training module 211 of FIG. 2 and can utilize one or more configured geometric loss functions in the process of training. In some embodiments, biological structure tokenizer module 1201 corresponds to trained tokenizers module 221 of FIG. 2 of biological language model service 201 of FIG. 2. In some embodiments, biological structure tokenizer module 1201 is utilized by a biological language model service such as biological language model service 111 of FIG. 1 and/or biological language model service 201 of FIG. 2 as part of a process for performing biological language reasoning.

In some embodiments, structure input processing module 1211 is a processing module for processing provided structure data. For example, structure information can be provided in a non-tokenized format, such as a user-accessible format that allows users to interface with and in a biological language more easily. For example, for a biological protein language model, the local structure of amino acids can be provided in a standardized and/or documented structure format. In some embodiments, the received structure input utilizes backbone frames for each amino acid including specifying relevant atoms of an amino acid based on their three-dimensional spatial coordinates. In some embodiments, the received structure input includes all atomic coordinates for the relevant biological object, such as atoms of a protein. In various embodiments, structure input processing module 1211 receives the provided structure input and performs any necessary pre-processing before feeding the processed structure data to structure encoder module 1213. For example, structure input processing module 1211 can convert the received structure data into backbone coordinates, or another format, that are compatible with structure encoder module 1213.

In some embodiments, structure encoder module 1213 is a processing module for encoding biological structure data into a latent encoding. For example, structure encoder module 1213 can receive structure data such as backbone coordinates. When applied to a query protein, the backbone coordinates can correspond to the spatial coordinates of specific atoms of a specific amino acid of the protein. Other formats can be used as well, such as formats the include all atomic coordinates for eventually generating all-atom structure latents or tokens. In various embodiments, structure encoder module 1213 maps the received structure data into a lower-dimensional latent space. The generated structure latents are encoded with context information associated with local structure such as the local structure of protein amino acids relative to their nearest neighboring amino acids. In various embodiments, structure encoder module 1213 utilizes geometric reasoning module 1215 to perform geometric attention on the structure data as part of the encoding process.

In some embodiments, geometric reasoning module 1215 is a processing module for performing geometric reasoning including a geometric form of self-attention on structure data. For example, for protein structure, geometric reasoning module 1215 can encode the structure of local amino acids based on their relative distance and direction to neighboring local amino acids. In some embodiments, the neighboring amino acids are determined by physical distance allowing geometric reasoning module 1215 to encode complex physical structure properties of proteins at a local level. For example, using direction and distance factors between local neighboring amino acids, self-attention scores can be determined. In some embodiments, the determined direction properties are attenuated based on the determined distance properties when determining self-attention scores. In various embodiments, geometric reasoning module 1215 utilizes a geometric attention mechanism block in addition to other functional components such as layer normalization and feed forward blocks.

In some embodiments, quantizing module 1217 is a processing module for quantizing structure latents. For example, the latent values encoded by structure encoder module 1213 are quantized by quantizing module 1217 to determine structure tokens. In various embodiments, quantizing module 1217 discretizes the structure latents generated by structure encoder module 1213 using a learned codebook. For example, similar latents can be mapped to similar codewords corresponding to biological structure tokens using clustering and/or vector quantization techniques. In some embodiments, quantizing module 1217 uses a vector quantizer to create tokens from structure latents.

In some embodiments, structure decoder module 1219 is a processing module for decoding biological structure tokens back into their original structure data space. For example, structure decoder module 1219 can map the lower-dimensional latent space used by structure tokens back to the structure data space used for the input to structure encoder module 1213. When applied to a query protein, the decoded results can be backbone coordinates that correspond to the spatial coordinates of specific atoms of specific amino acids of the predicted protein. In some embodiments, the decoded results can include coordinates for all relevant or all atoms of a biological object such as all atoms of a query protein. In some embodiments, an additional post-processing step is performed to convert the decoded structure data to a format used as input to structure input processing module 1211.

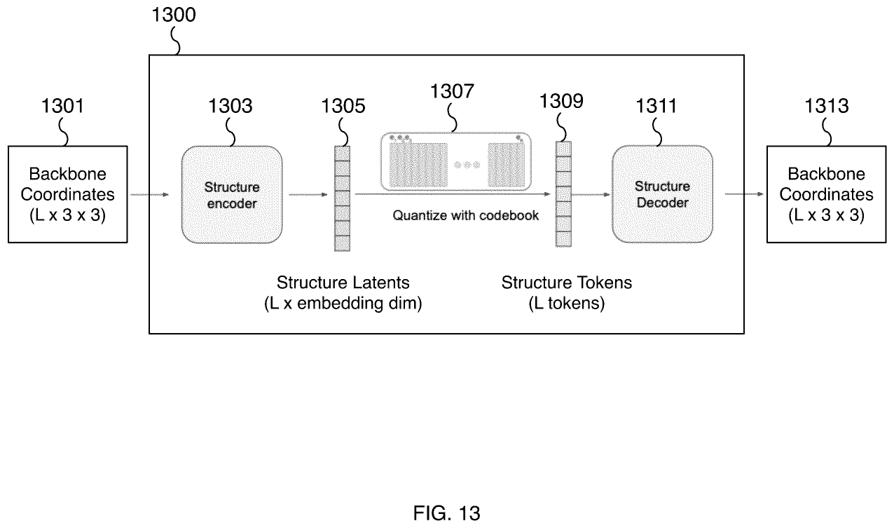

FIG. 13 is a block diagram illustrating an embodiment of a tokenizer for biological structure. In the example shown, structure tokenizer 1300 is an auto-encoder that can encode biological structure data into biological structure tokens and decode into biological structure tokens back to biological structure data. The encoded biological structure tokens are encoded with context information associated with local biological structure such as the local structure of protein amino acids relative to their nearest neighboring amino acids. In some embodiments, structure tokenizer 1300 is a variational autoencoder with vector quantization (VQ-VAE). As shown in FIG. 13, backbone coordinates 1301 are provided to structure encoder 1303, which generates structure latents 1305. Structure latents 1305 are quantized with quantizing module 1307 to generate structure tokens 1309. Structure tokens 1309 can be decoded by structure decoder 1311 to backbone coordinates 1313. In some embodiments, structure tokenizer 1300 corresponds to biological structure tokenizer module 1201 of FIG. 12, structure encoder 1303 corresponds to structure encoder module 1213 of FIG. 12, quantizing module 1307 corresponds to quantizing module 1217 of FIG. 12, and structure decode 1311 corresponds to structure decoder module 1219 of FIG. 12. In various embodiments, backbone coordinates 1301 are the output generated by structure input processing module 1211 of FIG. 12. In various embodiments, structure encoder 1303 can include a geometric reasoning block for encoding local structure. The geometric reasoning block can correspond to geometric reasoning module 1215 of FIG. 12.

In various embodiments, backbone coordinates 1301 and 1313 utilize a coordinates format for describing biological structure such as protein structure. In the example of FIG. 13, the structure data of backbone coordinates 1301 and 1313 each include backbone coordinates for a protein with L amino acids. Each of the L amino acids of the example protein have a local structure defined by three corresponding backbone atoms. The backbone atoms can correspond to nitrogen (N), alpha-carbon (CA), and carbon (C) atoms and each can be described by a set of three-dimensional spatial coordinates corresponding to X, Y, and Z coordinates. For a protein with L amino acids, the backbone coordinates have size L×3×3. When backbone coordinates 1301 are tokenized, the output is structure tokens 1309, which includes L tokens, one for each amino acid. When structure tokens 1309 are decoded, the output is decoded to backbone coordinates 1313. Although specific spatial and structure formats are described, alternative formats are appropriate as well. For example, structure encoder 1303 and structure decoder 1311 can utilize a different structure format such as a frame format that selects a single backbone atom as the origin and a rotation matrix based on the frame defined by the nitrogen (N), alpha-carbon (CA), and carbon (C) backbone atoms. As another example, in some embodiments, structure encoder 1303 and structure decoder 1311 can utilize a format that includes all atoms and all corresponding atomic coordinates for a biological object for encoding all-atom structure tokens.

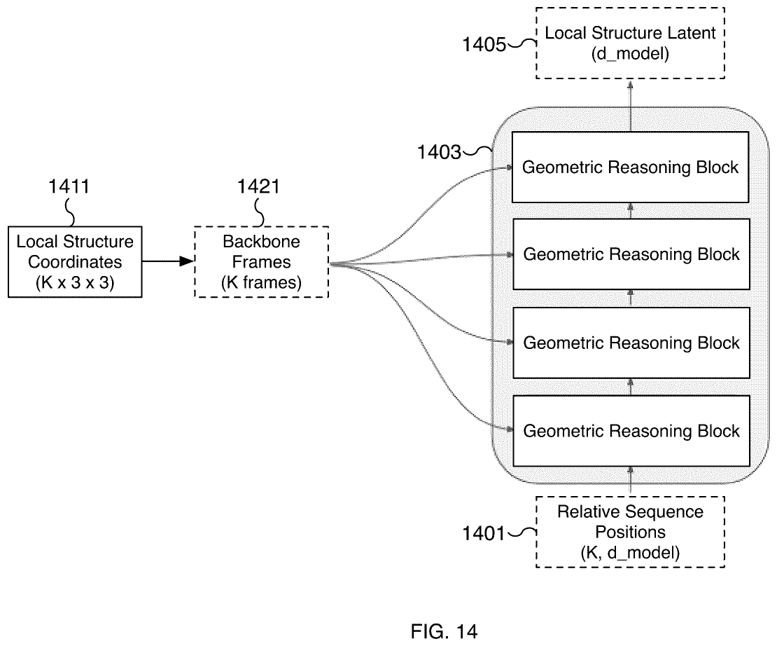

FIG. 14 is a block diagram illustrating an embodiment of a structure encoder of a biological structure tokenizer. In the example shown, structure encoder 1403 is a component of a biological structure tokenizer that encodes geometric biological structure. Structure encoder 1403 includes multiple instances of chained geometric reasoning blocks to apply geometric reasoning including geometric attention. Structure encoder 1403 receives relative sequence positions 1401 as input along with backbone frames 1421. Backbone frames are generated from local structure coordinates 1411. Structure encoder 1403 outputs local structure latent 1405. As shown in FIG. 14, the operation of structure encoder 1403 is applied to a single local reference target such as a query amino acid of a protein. In practice, structure encoder 1403 is applied to an entire query target (such as a query protein) and each query point (such as a query amino acid) can be processed in parallel. Although shown and described in FIG. 14 with respect to a particular local structure and backbone frames, structure encoder 1403 of a biological structure tokenizer can utilize other formats including all-atom formats for generating structure tokens. For example, in some embodiments, a structure encoder can generate all-atom structure tokens.

In some embodiments, local structure coordinates 1411 uses a coordinate format for local structure and includes local coordinates for K amino acids, each with three backbone atoms and each backbone atom with three coordinates (corresponding to X, Y, and Z coordinates). Depending on convention, the K amino acids can include the query point, such as the selected amino acid of the query protein. For example, for K=16, the query point and 15 other amino acids are described. The number K can be a hyperparameter and may have a configured value such as 16, 24, 32, etc., as appropriate. As shown in FIG. 14, local structure coordinates 1411 has size K×3×3. In some embodiments, local structure coordinates 1411 is converted to backbone frames 1421. For example, the K neighboring amino acids are converted to K frames, one for each of the K neighboring amino acids. The frame format can utilize an origin point and a rotation matrix. In various embodiments, structure encoder 1403 utilizes the frame format and receives backbone frames 1421 at each geometric reasoning block.

In some embodiments, relative sequence positions 1401 includes a vector that describes the relative position of the K amino acids of local structure coordinates 1411. For example, relative sequence positions 1401 can include K relative sequence position values with a second dimension based on the dimensions of the model (d_model). In some embodiments, a relative sequence position is a location offset and is based on the offset of a neighboring amino acid from the selected amino acid using the protein sequence for position ordering. For example, using the selected amino acid as a reference point, the selected amino acid has a relative sequence position with value 0, the previous amino acid has a relative sequence position with value −1, and the next amino acid has a relative sequence position with value 1. A subset of the K values of relative sequence positions 1401 may have relative sequence position values such as −3, −2, −1, 0, 1, 2, 3, 67, 68, −91, −92. In some embodiments, the sequence position values including the relative sequence position values are binned values. For example, relative sequence position values within than certain threshold range can be binned together.

In some embodiments, structure encoder 1403 corresponds to structure encoder module 1213 of FIG. 12 and/or structure encoder 1303 of FIG. 13. In some embodiments, local structure coordinates 1411 corresponds to at least a subset of coordinates of backbone coordinates 1301 of FIG. 13 and local structure latent 1405 corresponds to a latent result of structure latents 1305 of FIG. 13. In various embodiments, structure encoder 1403 may include additional components that are not shown.

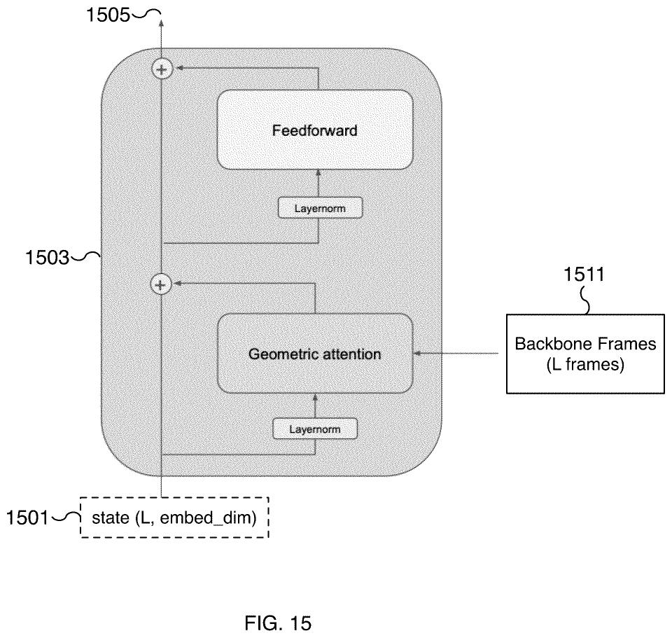

FIG. 15 is a block diagram illustrating an embodiment of a geometric reasoning block of a biological structure tokenizer. In the example shown, geometric reasoning block 1503 is a component of a structure encoder of a biological structure tokenizer. Geometric reasoning block 1503 receives as input state information 1501 and backbone frames 1511. Geometric reasoning block 1503 includes multiple functional components including layer normalization, geometric attention, and feed forward blocks to determine updated state 1505. The arrangement of the functional components including layer normalization, geometric attention, and feed forward blocks of geometric reasoning block 1503 determines an updated representation of the state 1501 and is outputted as updated state 1505. In various embodiments, updated state 1505 reflects the application of geometric attention and feed forward networks.

In particular, the geometric attention block of geometric reasoning block 1503 includes geometric attention mechanisms to encode local geometric understanding. For example, for protein structure, the geometric attention mechanisms can encode an understanding of the local structure of each amino acid of a protein. In some embodiments, the geometric attention block determines local structure understanding for each amino acid based on the distance and direction of neighboring amino acids. In some embodiments, geometric reasoning block 1503 is utilized by geometric reasoning module 1215 of FIG. 12, structure encoder 1303 of FIG. 13, and/or structure encoder 1403 of FIG. 14. For example, structure encoder 1403 of FIG. 14 includes multiple instances of geometric reasoning block 1503.

In various embodiments, geometric reasoning block 1503 processes input state information 1501 and backbone frames 1511 for a biological object described by L positions of a sequence state, such as a protein described by L amino acids. For a specific position of the L positions of input state information 1501, the particular state information received can include local structure information such as the neighborhood context associated with that position. In some embodiments, the neighborhood context (not shown) includes as description of the K nearest neighbors, such as the K physically neighboring amino acids when describing the local structure of a specific amino acid of a protein. Additionally, each of the L positions of the biological object, such as each of the L amino acids of a protein, are described by one of the corresponding L frames of backbone frames 1511. Although shown and described in FIG. 15 with respect to backbone frames, in some embodiments, geometric reasoning block 1503 utilizes another format such as an all-atom format.

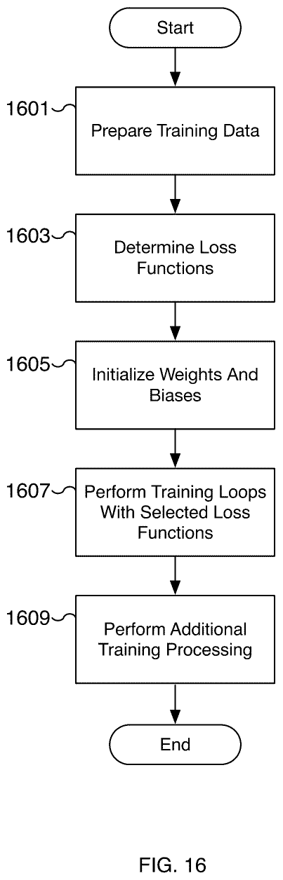

FIG. 16 is a flow chart illustrating an embodiment of a process for training a biological structure tokenizer. For example, using the process of FIG. 16, a tokenizer for use with a biological language reasoning model is trained to encode biological structure including local structure into structure tokens. In some embodiments, the structure tokenizer utilizes geometric reasoning including geometric attention mechanisms to learn biological structure. The trained tokenizer can be used to tokenize training data for training a biological language reasoning model and/or for performing inference using the trained biological language reasoning model. In some embodiments, the biological structure tokenizer is an autoencoder and the associated decoder is used to decode structure tokens into biological structure. In some embodiments, the process of FIG. 16 is used to train a biological structure tokenizer including to determine the trained weights, biases, and/or codebook of biological structure tokenizer module 1201 of FIG. 12 and/or structure tokenizer 1300 of FIG. 13. In some embodiments, the process of FIG. 16 utilizes geometric reasoning module 1215 of FIG. 12 and/or geometric reasoning block 1503 of FIG. 15 to encode geometric biological structure including local geometric biological structure. In some embodiments, the process of FIG. 16 is performed at 701 of FIG. 7 and/or at 803 of FIG. 8 as part of the process of preparing tokenizers for performing biological language reasoning.

At 1601, training data is prepared. For example, the input data is preprocessed to prepare training data for the structure tokenizer. In some embodiments, the data is structure data such as protein structure data and the data is prepared into a format such as a format that extracts backbone atom coordinates. In some embodiments, the input data processed at 1601 corresponds to backbone coordinates 1301 of FIG. 13. In some embodiments, the input data is processed to prepare training data at least in part by determining a frame of reference for local structure, such as local structure of amino acids of a protein. In some embodiments, the training data is prepared using all atomic coordinates, for example, to train the biological structure tokenizer for generating all-atom structure tokens.

At 1603, one or more loss functions are determined. For example, one of more loss functions including geometric loss functions are selected for use during the training process. The determined geometric loss functions can be used during training to evaluate error measurements between input and reconstructed output. In various embodiments, geometric loss functions are selected to optimize the ability for the tokenizer to encode biological structure into latent geometric structure representations, to quantize the encoded structure latents into structure tokens, and to decode structure tokens back into a biological structure space of the input data. Different loss functions including different geometric loss functions can be utilized including at different phases of the training process. For example, a geometric loss function can be used for computing loss related to geometric distance and another for loss related to geometric direction. As another example, different geometric loss functions that classify geometric loss based on bins can be utilized, for example, early in the training cycle when the error loss can be large. For example, a distogram-based geometric loss function can be used to bin the relative distance between amino acids and/or an anglogram-based geometric loss function can be used to bin the relative direction or orientation of amino acids.

In some embodiments, a geometric distance loss function is used. For example, a geometric distance loss function can be used during training for structure prediction including for the prediction of structure coordinates such as protein structure coordinates. In some embodiments, the geometric distance loss is computed based on pairwise distances between backbone atoms, such as the backbone atoms of amino acids for a protein. For example, pairwise distances are computed between all pairs of atoms in the ground truth structure. The resulting matrix has dimension 3L×3L corresponding to three backbone atoms per position and the L sequence length of the protein. The same matrix for the predicted structure is computed and an error loss between the two matrices is determined. In some embodiments, the error loss computed is a mean squared error loss between the two matrices. Although the previous example is described with respect to protein backbone atoms and an example corresponding 3L×3L resulting matrix, the approach can be extended to any number of atoms in the structure. For example, the geometric distance loss function can be applied to all side chain atoms in a protein structure, or for interactions with other biological molecules.

In some embodiments, a geometric direction loss function is used. For example, a geometric direction loss function can be used during training for structure prediction including for the prediction of structure coordinates such as protein structure coordinates. In some embodiments, the geometric direction loss is modeled using a function based on the relative orientations of bond vectors in the predicted and ground truth structures and is applicable to any biological molecule. For example, the bond vectors and Z-axis vectors for all selected atoms in the ground truth and predicted structures can be computed. The Z-axis vectors can be defined as the cross product between any two bonds for a given atom. In the event an atom has fewer than two bonds, no Z-axis is computed. Next, the dot products between all pairs of vectors in the ground truth structure are computed. The resulting matrix has dimensions approximately 2*N, where N corresponds to the number of atoms in the ground truth structure. In various embodiments, N is an approximation since, for example, not all atoms may have defined Z-axis vectors. The corresponding dot product computations are repeated for the predicted structure vectors and an error loss between the ground truth and predicted structure matrices is determined. In some embodiments, the error loss computed is a mean squared error loss between the two matrices. In various embodiments, when the geometric direction loss function is applied to protein structure, the backbone vectors and Z-axis vectors for all residues in the ground truth and predicted structures are computed. Next, the dot products between all pairs of vectors in the ground truth structure are computed. The resulting matrix has dimensions 6L×6L, where the protein has a sequence length of L. The dot product computations are repeated for the predicted structure vectors and an error loss between the ground truth and predicted structure matrices is determined.

In some embodiments, the computed backbone vectors for the geometric direction loss function correspond to the vectors between the three backbone atoms of a residue. The backbone atoms can correspond to nitrogen (N), alpha-carbon (CA), and carbon (C) atoms and each can be described by a set of three-dimensional spatial coordinates corresponding to X, Y, and Z coordinates. In some embodiments, the three backbone vectors are the vectors defined by the nitrogen and alpha-carbon atoms (N→CA), the alpha-carbon and carbon atoms (CA→C), and the carbon and nitrogen atoms (C→N). The three additional Z-axis vectors can be determined by the cross-product applied to each different pair of backbone vectors. In some embodiments, the three Z-axis vectors correspond to the vectors (N→CA)×(CA→C), (CA→C)×(C→N), and (C→N)×(N→CA). Although the geometric direction loss function is described above with six specific vectors, other sets of vectors such ones relying on the X-axis and/or Y-axis vectors or another combination of vectors can be used as well.

In some embodiments, a geometric loss function can be used that relies on determining three vectors for each residue. For example, an anglogram-based or binned direction classification geometric loss function can be used during training for structure prediction including for the prediction of structure coordinates such as protein structure coordinates. In some embodiments, the geometric loss is computed based on computing two backbone vectors and a third orthogonal vector such as the Z-axis vector that can be determined by the cross-product of the two selected backbone vectors. In some embodiments, the three vectors are centered around the alpha-carbon atom and correspond to the vectors (N→CA), (CA →C), and (N→CA)×(CA→C). In some embodiments, the geometric loss is computed based on the relative orientations of the three vectors in the predicted and ground truth structures. For example, when applied for protein structure, the two backbone vectors and the Z-axis vector are computed for the ground truth structure. Next, the cosine similarity between all pairs of vectors is computed. The resulting matrix has dimensions 3L×3L, where the protein has a sequence length of L. The similarities are then binned into, for example, 16 bins, and each of the bins is then classified. In some embodiments, the anglogram-based geometric loss function provides improved results particularly when applied early in the training cycle when the determined geometric loss can be large. Although described above with respect to two particular backbone vectors and a corresponding Z-axis vector, another set of three appropriate vectors to define a residue can be used as well.

At 1605, the weights and biases of the tokenizer are initialized. For example, the weights and biases of the encoder, decoder, and quantizer are initialized. The values for the weights and biases may be initialized with a random initialization technique.

At 1607, training loops are performed with the selected loss functions. For example, multiple iterations of a training loop are performed to train the biological structure tokenizer. In some embodiments, the training process utilizes geometric reasoning including geometric attention mechanisms and the loss functions selected at 1603. For example, the weights and biases initialized at 1605 are updated during each pass through the training loop and the selected loss functions are applied to minimize loss. In various embodiments, the structure tokenizer includes a quantization step and the quantizer is updated during the training process. In some embodiments, the training loops are performed until a target error rate is reached and/or for a configured number of epochs.

At 1609, additional training processing is performed. For example, additional processing can be performed after the completion of the main training loops at 1607. In some embodiments, the additional processing includes additional fine-tuning and/or additional processing to the structure tokenizer to improve its performance. In some embodiments, an additional processing step includes storing the trained weights, biases, and codebook results and/or deploying the trained structure tokenizer for use in performing biological language reasoning tasks.

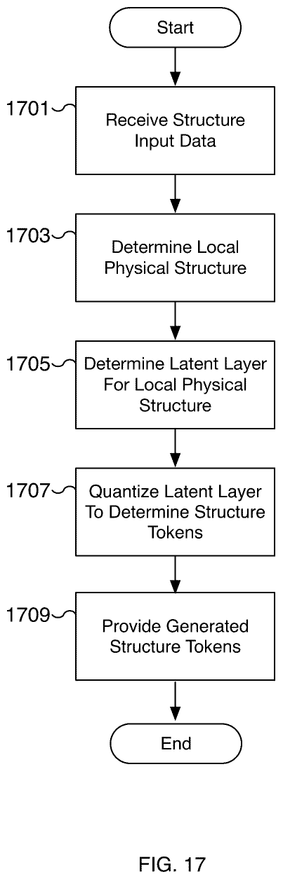

FIG. 17 is a flow chart illustrating an embodiment of a process for tokenizing biological structure. For example, using the process of FIG. 17, a structure tokenizer is applied to generate structure tokens that encode biological structure including local structure from biological structure input data. The generated structure tokens can be used for training and/or performing inference with respect to a biological language reasoning model. In some embodiments, the tokenizing is performed for a specific biological domain such as for a specific protein or query protein to encode the biological structure of the protein including its local structure with respect to the amino acids of the protein. In some embodiments, the structure tokenizer utilizes geometric reasoning including geometric attention mechanisms. In some embodiments, the biological structure tokenizer is an autoencoder and the associated decoder is used to decode structure tokens into a decoded format of biological structure data. In some embodiments, the process of FIG. 17 is performed at 701, 703, and/or 707 of FIG. 7, at 805 of FIG. 8, at 903 of FIG. 9, at 1005 of FIG. 10, at 1105 of FIG. 11, and/or at various steps when structure data requires tokenizing for biological language reasoning. In some embodiments, the process of FIG. 17 is performed by biological structure tokenizer module 1201 of FIG. 12 and/or structure tokenizer 1300 of FIG. 13. In some embodiments, the process of FIG. 17 is performed using geometric reasoning module 1215 of FIG. 12 and/or geometric reasoning block 1503 of FIG. 15 to encode geometric biological structure including local geometric biological structure.

At 1701, structure input data is received. For example, structure input data for a biological target such as a query protein is received. The data can be provided in a format that describes biological structure such as with coordinates for backbone atoms. For example, when applied to protein structure, the coordinates of backbone atoms for each specified amino acid of a protein can be received. Alternative formats are appropriate as well based and can vary depending on the trained structure tokenizer. For example, in some embodiments, the format utilizes an all-atom format for generating all-atom structure tokens. In some embodiments, the structure input data format includes and/or utilizes backbone frames such as a frame format that describes local structure. For example, a frame can describe an origin, such as the coordinates of an origin for a specific backbone atom, and a corresponding rotation matrix to orient the local structure. In some embodiments, the structure input data corresponds to backbone coordinates 1301 of FIG. 13 and/or local structure coordinates 1411 of FIG. 14.

At 1703, the local physical structure is determined. For example, the local physical structure of the biological target is determined. In various embodiments, determining the local structure includes identifying local reference points and the corresponding surrounding structure of each reference point. For example, for a target protein, the local structure can be determined using each amino acid of the protein as a separate reference point. In various embodiments, the local structure of an amino acid of a protein can be based on its neighboring amino acids as determined by physical distance. Due to the physical structure of a protein, the determined neighboring amino acids typically differ from the amino acid sequence for the protein. Although described with respect to a protein, the local physical structure can be determined for the biological target using similar techniques.

At 1705, a latent layer is determined for the local physical structure. For example, structure latents for the local physical structure of the biological target are determined using the trained structure encoder of the structure tokenizer such as structure encoder module 1213 of FIG. 12, structure encoder 1303 of FIG. 13, and/or structure encoder 1403 of FIG. 14. In various embodiments, the determined structure latents encode local physical structure including neighboring structure for each local reference point. For example, for a target protein, structure latents can be determined for each described amino acid of the protein based on its neighboring amino acids. In various embodiments, the determined structure latents encode local geometric structure in a lower-dimensional latent space by applying geometric reasoning. In some embodiments, the determined structure latents correspond to structure latents 1305 of FIG. 13 and/or instances of local structure latent 1405 of FIG. 14.

At 1707, the latent layer is quantized to determine structure tokens. For example, the latent layer determined at 1705 is quantized using a quantizer or quantizing module such as quantizing module 1217 of FIG. 12 and/or quantizing module 1307 of FIG. 13 to determine a structure token for each local reference point. In various embodiments, structure tokens are created using a learned codebook. For example, similar latents can be mapped to similar codewords corresponding to biological structure tokens using clustering and/or vector quantization techniques. In some embodiments, the determined structure tokens correspond to structure tokens 1309 of FIG. 13.

At 1709, the generated structure tokens are provided. For example, the set of structure tokens determined at 1707, such as structure tokens for each amino acid of a query protein, are provided. The generated structure tokens can be used for training and/or for inference with a biological language reasoning model. For example, the generated structure tokens can be provided for preparing a structure track as part of a multi-track input, such as masked multi-track input 301 of FIG. 3, for a multi-track biological language model.

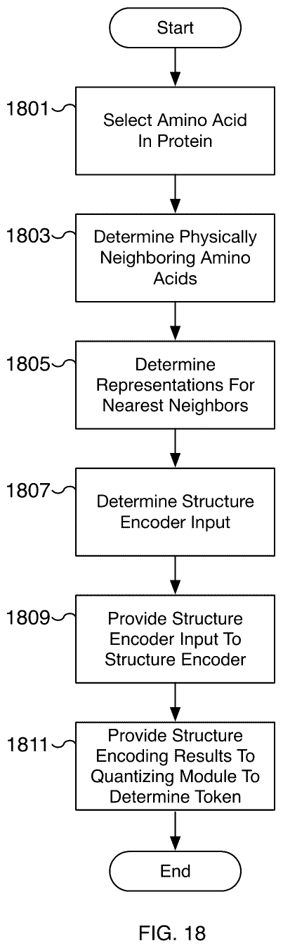

FIG. 18 is a flow chart illustrating an embodiment of a process for tokenizing a biological protein structure. For example, the process of FIG. 18 can be applied for each amino acid in a target (or query) protein to create a corresponding structure token. When applied to an entire protein, the set of corresponding structure tokens represents the entire protein's physical structure with latent encodings for local structure. The generated structure tokens can be used for training and/or performing inference with respect to a biological protein language reasoning model. In some embodiments, the structure tokenizer utilizes geometric reasoning including geometric attention mechanisms. In some embodiments, the protein structure tokenizer is an autoencoder and the associated decoder is used to decode protein structure tokens into a decoded format of protein structure data, for example, to visualize a generated protein.

In some embodiments, the process of FIG. 18 is performed at 701, 703, and/or 707 of FIG. 7, at 805 of FIG. 8, at 903 of FIG. 9, at 1005 of FIG. 10, at 1105 of FIG. 11, at 1703, 1705, 1707, and/or 1709 of FIG. 17, and/or at various steps when protein structure data requires tokenizing for biological language reasoning. In some embodiments, the process of FIG. 18 is performed by biological structure tokenizer module 1201 of FIG. 12 and/or structure tokenizer 1300 of FIG. 13. In some embodiments, the process of FIG. 18 is performed using geometric reasoning module 1215 of FIG. 12 and/or geometric reasoning block 1503 of FIG. 15 to encode geometric biological structure including local geometric biological structure.

At 1801, an amino acid in a protein is selected. For example, the process of FIG. 18 is performed over an entire protein for each amino acid described (or unmasked) for the protein. At 1801, a specific amino acid in a protein is selected for tokenization. In various embodiments, the process of FIG. 18 can be performed in parallel for each selected protein.

At 1803, physically neighboring amino acids are determined. For example, the nearest amino acids by physical distance to the amino acid selected at 1801 are selected. In some embodiments, the physically neighboring amino acids are determined by determining the distance between each specific amino acid and candidate amino acids of the protein. Based on the determined distance between a specific amino acid and a candidate amino acid, the candidate amino acid is included as one of the physically neighboring amino acids. In some embodiments, a total of K physically neighboring amino acids are selected, where the number K is configurable and can be a hyperparameter. In various embodiments, the physical distance can be determined by calculating the distance, such as the Euclidean distance, between reference points for each amino acid pair. For example, a reference location can be selected for a specific amino acid and a candidate amino acid. The reference location of an amino acid can include a coordinate for an origin location and a corresponding rotation matrix for the amino acid. In some embodiments, the origin location corresponds to the coordinates of a nitrogen (N), alpha-carbon (CA), or carbon (C) atom of the amino acid. Based on the selected reference locations, a distance is determined between an amino acid pair and used to determine whether a candidate amino acid is one of the nearest neighbors of the selected amino acid. Other techniques for determining the physically neighboring amino acids are appropriate as well. In various embodiments, the physically neighboring amino acids can include amino acids from another protein such as a binder protein or a binding target protein.

At 1805, representations for the physically neighboring amino acids are determined. For example, representations for the physically neighboring amino acids determined at 1803 are generated. In some embodiments, the representations utilize a frame format for each backbone atom of the amino acid. For example, a frame format for an amino acid can include the coordinates of the alpha-carbon atom of the amino acid and a 3×3 rotation matrix for the frame defined by the nitrogen, alpha-carbon, and carbon atoms of the amino acid. In some embodiments, the alpha-carbon is placed at the origin and the nitrogen atom defines the X-axis. Although specific spatial and structure formats are described, alternative representation formats are appropriate as well. For example, another reference point other than the nitrogen atom of a backbone can be used to define the X-axis. In some embodiments, the representations for the physically neighboring amino acids include a relative sequence position of each neighboring amino acid relative to the selected (or query) amino acid. For example, the representations for the physically neighboring amino acids can include a vector of sequence positions. In some embodiments, a relative sequence position is a location offset based on the offset of a neighboring amino acid from the selected amino acid using the protein sequence for position ordering. For example, using the selected amino acid as a reference point, the selected amino acid has a relative sequence position with value 0, the previous amino acid has a relative sequence position with value −1, and the next amino acid has a relative sequence position with value 1. In some embodiments, the determined representations for the physically neighboring amino acids correspond to relative sequence positions 1401 of FIG. 14, local structure coordinates 1411 of FIG. 14, and/or backbone frames 1421 of FIG. 14.

At 1807, input for a structure encoder is determined. For example, using the determined representations for the physically neighboring amino acids determined at 1805, a structure encoder input is determined. The determined input can include backbone frames for the physically neighboring amino acids and the relative sequence position for the physically neighboring amino acids. In some embodiments, the structure encoder input includes the selected (or query) amino acid as a neighboring amino acid, for example, with a relative sequence position with value 0. In some embodiments, the structure encoder input includes relative sequence positions 1401 of FIG. 14. In some embodiments, the structure encoder input is based on relative sequence positions 1401 of FIG. 14 and backbone frames 1421 of FIG. 14. In some embodiments, the structure encoder input includes all atomic coordinates, for examples, for generating all-atom structure tokens.

At 1809, structure encoder input is provided to the structure encoder. For example, the structure encoder input determined at 1807 is provided to the structure encoder of the trained structure tokenizer. The structure encoder encodes the provided input into one or more structure latent values. In various embodiments, the structure encoder applies one or more geometric reasoning blocks to encode local structure. In some embodiments, the structure encoder corresponds to structure encoder module 1213 of FIG. 12, structure encoder 1303 of FIG. 13, and/or structure encoder 1403 of FIG. 14.

At 1811, the structure encoding results are provided to a quantizing module for tokenization. For example, the structure latent results determined at 1809 by the structure encoder are provided to a quantizing module for quantization. In some embodiments, the quantizing module is quantizing module 1217 of FIG. 12 and/or quantizing module 1307 of FIG. 13. The output of the quantizing module is a structure token for the amino acid selected at 1801. In some embodiments, the structure token is created using a learned codebook. In some embodiments, the determined structure token is a structure token of structure tokens 1309 of FIG. 13.

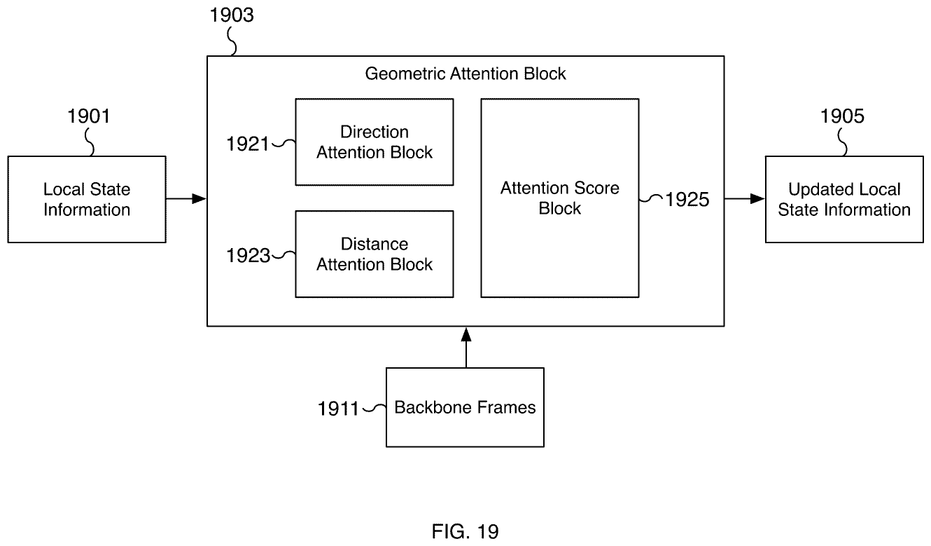

FIG. 19 is a block diagram illustrating an embodiment of a geometric attention block for performing geometric attention on biological structure. For example, geometric attention block 1903 is trained to determine and apply geometric attention using local state information 1901 and backbone frames 1911 as inputs. Within geometric attention block 1903, direction attention block 1921 and distance attention block 1923 determine intermediary direction and distance attention score results using local state information 1901 and backbone frames 1911. The attention results of direction attention block 1921 and distance attention block 1923 are used by attention score block 1925 to determine a geometric attention result. As a result, local state information 1901 is updated using the determined geometric attention result and outputted as updated local state information 1905. In various embodiments, updated local state information 1905 includes a geometric attention result with geometric attention scores and an updated sequence state with updated weighted sequence representations. Although shown and described in FIG. 19 with respect to backbone frames, in some embodiments, geometric reasoning block 1903 utilizes another format such as an all-atom format.

In various embodiments, geometric attention block 1903 can be one of multiple geometric attention blocks that are chained together to apply geometric attention. In some embodiments, the geometric attention is applied to protein structure to encode local protein structure information. In some embodiments, geometric attention block 1903 corresponds to the geometric attention mechanism used by transformer block with geometric attention 541 of FIG. 5, transformer block with a geometric attention 603 of FIG. 6, and/or geometric reasoning block 1503 of FIG. 15.

In some embodiments, geometric attention block 1903 is utilized by geometric reasoning module 415 of FIG. 4 and/or geometric reasoning module 1215 of FIG. 12. In some embodiments, a biological language reasoning model such as the model corresponding to or associated with biological language model service 111 of FIG. 1, biological language model service 201 of FIG. 2, biological language model 303 of FIG. 3, and/or biological language model module 401 of FIG. 4 incorporates one or more instances of geometric attention block 1903. In some embodiments, a structure encoder such as structure encoder 1303 of FIG. 13 and/or structure encoder 1403 of FIG. 14 incorporates one or more instances of geometric attention block 1903.

As shown in FIG. 19, geometric attention block 1903 includes components direction attention block 1921, distance attention block 1923, and attention score block 1925. Other components of geometric attention block 1903 may exist but are not shown. In various embodiments, direction attention block 1921 is an attention block that determines an attention score based on the direction a local query object of a target object is to its local neighbors. For example, the direction (or orientation) of a query amino acid of a protein is compared to its neighboring amino acids. Similarly, distance attention block 1923 is an attention block that determines an attention score based on the distance a local query object of a target object is to its neighbors. For example, the distance between a query amino acid of a protein from each of its neighboring amino acids is determined. In various embodiments, distance attention block 1923 and attention score block 1925 each determine and utilize corresponding direction and distance, query and key vectors, respectively, for example, to apply separate learning parameters when determining attention scores.

In various embodiments, the determined direction and distance attention results of direction attention block 1921 and distance attention block 1923, respectively, are processed by attention score block 1925. For example, attention score block 1925 can determine a weighted attention score by applying learned and/or configured weights to direction attention and distance attention results. In various embodiments, a value vector is used to compute the geometric attention result. In some embodiments, rotation matrices are applied to the value vector and inverse rotation matrices are applied to the geometric attention result so that each is in alignment with the appropriate frame of reference. In some embodiments, the weighted attention scores undergo a normalization process as part of determining the geometric attention result.

In some embodiments, local state information 1901 can be initialized to include local state information, such as local state information for each amino acid in a protein. The included local information for a specific amino acid can include references to its neighboring amino acids. For example, a list of a specific amino acid's neighbors based on their offsets in the protein sequence can be included in an initial version of local state information 1901. In some embodiments, local state information 1901 corresponds to and/or reflects an updated version of state information 601 of FIG. 6 and/or relative sequence positions 1401 of FIG. 14.

In some embodiments, backbone frames 1911 includes local physical structure information, such as local amino acid structure information using a frame format. For example, a backbone frame can specify an origin and rotation matrix for a specific amino acid based on the backbone atoms of the amino acid. In some embodiments, backbone frames 1911 corresponds to backbone frames 521 of FIG. 5, backbone frames 621 of FIG. 6, and/or backbone frames 1421 of FIG. 14.

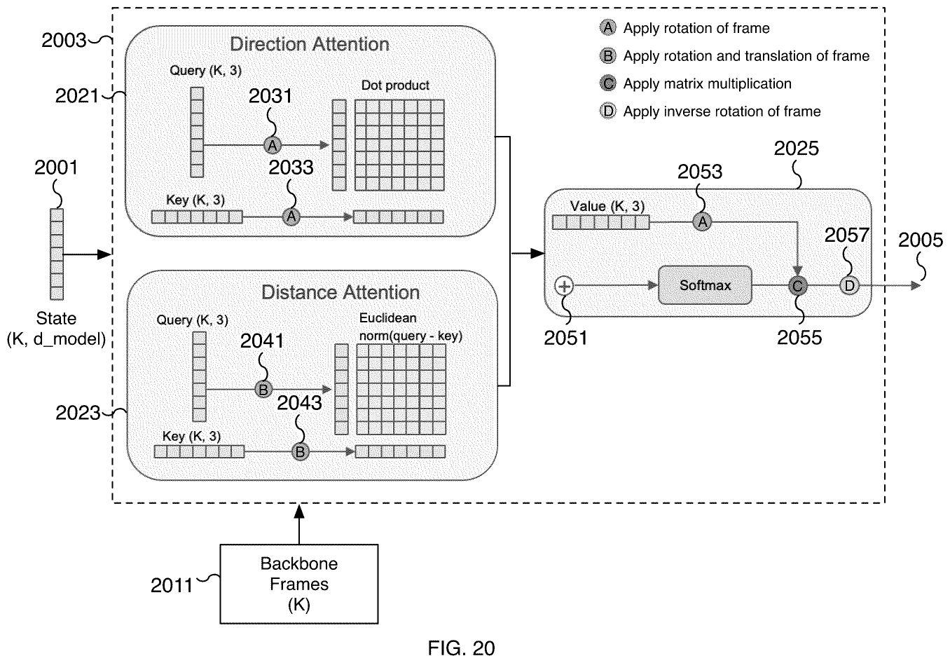

FIG. 20 is a block diagram illustrating an embodiment of a geometric attention block for performing geometric attention on biological structure. For example, geometric attention block 2003 is trained to determine and apply geometric attention using local state information 2001 and backbone frames 2011 as inputs. Within geometric attention block 2003, direction attention block 2021 and distance attention block 2023 determine intermediary direction and distance attention score results using local state information 2001 and backbone frames 2011. The attention results of direction attention block 2021 and distance attention block 2023 are used by attention score block 2025 to determine a geometric attention result. Using the determined geometric attention result, local state information 2001 is updated by attention score block 2025 and outputted as updated local state information 2005. In some embodiments, geometric attention block 2003 is geometric attention block 1903 of FIG. 19 with additional implementation details for performing geometric attention. For example, local state information 2001 is local state information 1901 of FIG. 19, backbone frames 2011 is backbone frames 1911 of FIG. 19, direction attention block 2021 is direction attention block 1921 of FIG. 19, distance attention block 2023 is distance attention block 1923 of FIG. 19, attention score block 2025 is attention score block 1925 of FIG. 19, and/or updated local state information 2005 is updated local state information 1905 of FIG. 19. As shown in the legend of FIG. 20, computational operations 2031, 2033, and 2053 are denoted with the letter A and each corresponds to frame rotation operations, computational operations 2041 and 2043 are denoted with the letter B and each corresponds to combined frame rotation and translation operations, computational operation 2055 is denoted with the letter C and corresponds to matrix multiplication operations, and computational operation 2057 is denoted with the letter D and corresponds to frame inverse rotation operations. Although shown and described in FIG. 20 with respect to backbone frames, in some embodiments, geometric reasoning block 2003 utilizes another format such as an all-atom format.

In some embodiments, direction attention block 2021 receives local state information 2001 and backbone frames 2011 as inputs. Local state information 2001 is used by direction attention block 2021 to create direction query and direction key vectors. Computational operations 2031 and 2033 are applied to direction query and direction key vectors, respectively, to rotate the direction query and direction key vector elements by the rotation transformations of their respective frames using rotation transformations from backbone frames 2011. In various embodiments, computational operations 2031 and 2033 each corresponds to matrix multiplication operations to apply rotation transformations. For example, the K frames of backbone frames 2011 can include corresponding 3×3 rotation matrices that are applied position-wise to the appropriate elements of local state information 2001. In particular embodiments, the dimension K corresponds to the number of local neighbors, such as the K amino acids in the local physical structure context of a specific amino acid. The resulting direction query and direction key vectors are used to determine a direction attention result. In some embodiments, the direction attention result scores are determined using dot product operations on direction query and direction key vector elements. Direction attention block 2021 provides the direction attention result to attention score block 2025.

In some embodiments, distance attention block 2023 receives local state information 2001 and backbone frames 2011 as inputs. Local state information 2001 is used by distance attention block 2023 to create distance query and distance key vectors. Computational operations 2041 and 2043 are applied to distance query and distance key vectors, respectively, to rotate and translate the distance query and distance key vector elements by the rotation and translation transformations of their respective frames using rotation and translation transformations from backbone frames 2011. In various embodiments, computational operations 2041 and 2043 each corresponds to matrix multiplication and vector addition operations to apply rotation and translation transformations. For example, the K frames of backbone frames 2011 can include corresponding 3×3 rotation matrices and 3×1 translation vectors that are applied position-wise to the appropriate elements of local state information 2001. In particular embodiments, the dimension K corresponds to the number of local neighbors, such as the K amino acids in the local physical structure context of a specific amino acid. The resulting distance query and distance key vectors are used to determine a distance attention result. In some embodiments, the distance attention result scores are determined using Euclidean norm operations on the difference between corresponding distance query and distance key vector element values. Distance attention block 2023 provides the distance attention result to attention score block 2025.

In some embodiments, attention score block 2025 receives local state information 2001, backbone frames 2011, a direction attention result from direction attention block 2021, and a distance attention result from distance attention block 2023 as inputs. Attention score block 2025 outputs a geometric attention result that corresponds to updated local state information 2005. In various embodiments, attention score block 2025 determines a weighted attention result with weighted attention result computation 2051 based on direction attention and distance attention results, for example, to attenuate the direction attention result scores by the distance attention result scores. In some embodiments, intermediate weighted direction attention result and intermediate weighted distance attention result are determined by applying learned and/or configured direction and distance term weights, respectively, to the direction attention and distance attention results. The intermediate weighted distance result scores are then subtracted from the intermediate weighted direction result scores to determine a weighted attention result. For example, for a particular element of a weighted attention result, a weighted attention score for element_{i,j} can be determined with weighted attention result computation 2051 by computing element_{i,j}=weight_direction*direction_term −weight_distance*distance_term. In various embodiments, geometric attention block 2003 learns the per-head distance and direction term weights that are applied when computing weighted attention scores for a weighted attention result.

In various embodiments, the weighted attention result determined at weighted attention result computation 2051 is provided to a softmax module to apply a softmax function. For example, a softmax function is applied to normalize the weighted attention scores of the weighted attention result into a valid probability distribution. In parallel to the softmax module, attention score block 2025 creates a value vector using local state information 2001 and backbone frames 2011 as input. Computational operation 2053 is applied to the value vector to rotate the value vector elements by the rotation transformations of their respective frames using rotation transformations from backbone frames 2011. In various embodiments, computational operation 2053 corresponds to matrix multiplication operations to apply rotation transformations, and computational operation 2053 functions similar to computational operations 2031 and 2033 described above.

As shown in FIG. 20, the resulting transformed value vector of attention score block 2025 is used to determine a geometric attention result by applying computational operation 2055 and computational operation 2057. Computational operation 2055 performs a matrix multiplication using the normalized weighted attention result and the transformed value vector. Computational operation 2057 applies an inverse rotation transformation to the result of computational operation 2055 to transform the geometric attention result into the appropriate frame of reference. In various embodiments, the rotation transformation applied at computational operation 2057 corresponds to the inverse of the rotation transformation applied at computational operation 2055. In various embodiments, attention score block 2025 updates the local state with the computed geometric attention result and outputs the geometric attention result as updated local state information 2005. In various embodiments, updated local state information 2005 includes the determined geometric attention result with geometric attention scores and an updated sequence state with updated weighted sequence representations. In some embodiments, updated local state information 2005 is the input source to a downstream geometric attention block, for example, when multiple geometric attention blocks are chained together. In some embodiments, the chained functionality includes additional functional blocks such as layer normalization and feed forward blocks. In various embodiments, geometric attention block 2003 applies geometric attention to sequence state information received as local state information 2001 and outputs updated sequence state information as updated local state information 2005.

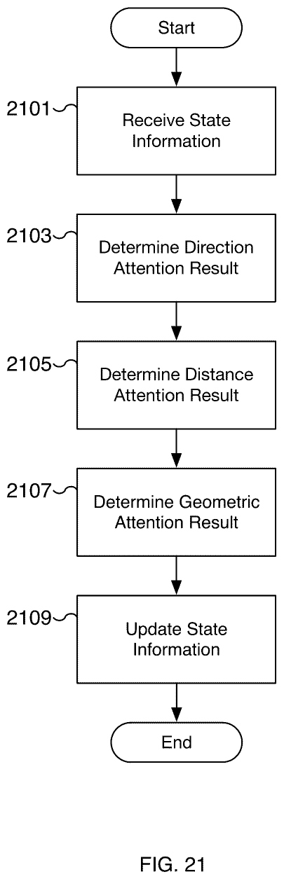

FIG. 21 is a flow chart illustrating an embodiment of a process for performing geometric attention on biological structure. For example, using the process of FIG. 21, a geometric attention block can determine geometric attention scores from local state and structure inputs. In some embodiments, the process of FIG. 21 updates state information based on a geometric attention result which can be forwarded to additional geometric attention blocks, as appropriate. The process of FIG. 21 can be performed as part of a process for tokenizing local structure for a biological domain such as for protein structure. By applying geometric attention to biological structure data, long-range geometric dependencies and/or contextual relationships between different elements within a biological object, such as between amino acids of a protein, can be captured. In some embodiments, the process of FIG. 21 is performed when performing biological language reasoning using biological structure data. In some embodiments, the process of FIG. 21 is performed by geometric attention block 1903 of FIG. 19 and/or geometric attention block 2003 of FIG. 20.

In some embodiments, the process of FIG. 21 is one of the processes performed by transformer block with geometric attention 541 of FIG. 5, transformer block with a geometric attention 603 of FIG. 6, and/or geometric reasoning block 1503 of FIG. 15. Similarly, the process of FIG. 21 can be one of the processes performed by geometric reasoning module 415 of FIG. 4 and/or geometric reasoning module 1215 of FIG. 12. In some embodiments, the process of FIG. 21 is performed by and/or when using a biological language reasoning model such as the model corresponding to or associated with biological language model service 111 of FIG. 1, biological language model service 201 of FIG. 2, biological language model 303 of FIG. 3, and/or biological language model module 401 of FIG. 4. Similarly, in some embodiments, the process of FIG. 21 is performed by a structure encoder such as structure encoder 1303 of FIG. 13 and/or structure encoder 1403 of FIG. 14.

At 2101, state information is received. For example, structure state information including local structure information for a biological object such as a protein is received. The received structure state information can include state sequence information and representations of a local neighboring structure and associated context. For example, for a biological protein object, the state information can include local state information for each amino acid in the protein. The included local information for a specific amino acid can include references to its neighboring amino acids. For example, a list of a specific amino acid's neighbors based on their offsets in the protein sequence can be included in an initial version of the state information received at 2101 such as a version of the state information before it has been updated with geometric attention. In some embodiments, the state information received at 2101 includes local physical structure information such as backbone frames. For a protein object, each included backbone frame can specify an origin and rotation matrix for a specific amino acid based on the backbone atoms of the amino acid. In some embodiments, the local structure information including all atomic coordinates for performing geometric attention on the biological structure. In some embodiments, the state information received at 2101 corresponds to local state information 1901 and backbone frames 1911 of FIG. 19 and/or local state information 2001 and backbone frames 2011 of FIG. 20.

At 2103, a direction attention result is determined. For example, direction attention scores can be determined based on learned direction relationships. When applied to a protein, direction attention scores can be determined based on the relationship between neighboring amino acids with respect to their directions (or orientations). In some embodiments, direction metrics can be determined by taking each pair of specific amino acids of a protein and transforming each into a shared frame of reference before comparing their respective directions. For example, the state information received at 2101 can describe each amino acid in its own frame of reference and further include a set of transformations to apply to transform each amino acid into a shared or global frame of reference. In various embodiments, the direction attention result is determined at least in part by capturing the direction relationship between neighboring amino acids such as the closet K amino acids to each specific amino acid of a protein. In various embodiments, query, key, and value vectors are used to determine a direction attention result. For example, a direction query and direction key vector can be used to determine a direction attention result. In some embodiments, the key vector is applied after the distance attention result determined at 2105 is used to attenuate the direction attention result.

At 2105, a distance attention result is determined. For example, distance attention scores can be determined based on learned distance relationships. When applied to a protein, distance attention scores can be determined based on the relationship between neighboring amino acids with respect to the distance between them. In some embodiments, direction metrics can be determined by taking each pair of specific amino acids of a protein and transforming each into a shared frame of reference before determining the distance between them. For example, the state information received at 2101 can describe each amino acid in its own frame of reference and further include a set of transformations to apply to transform each amino acid into a shared or global frame of reference. In various embodiments, the distance attention result is determined at least in part by capturing the distance relationship between neighboring amino acids such as the closet K amino acids to each specific amino acid of a protein. In various embodiments, query, key, and value vectors are used to determine a distance attention result. For example, a distance query and distance key vector can be used to determine a distance attention result. In some embodiments, the key vector is applied after the distance attention result is used to attenuate the direction attention result determined at 2103.

At 2107, a geometric attention result is determined. For example, a geometric attention result is determined using the direction attention result determined at 2103 and the distance attention result determined at 2105. In some embodiments, learned (or configured) weight terms are applied to the direction and distance attention results and then a weighted distance attention result is used to attenuate a weighted direction attention result. The resulting weighted attention result is normalized such as by applying a softmax function. A value vector is determined and applied to the normalized attention scores to determine a geometric attention result. In various embodiments, transformations are applied to the different elements, such as to the elements of the value vector and the geometric attention result to ensure that computational results are in the appropriate reference context and/or frame of reference. In various embodiments, the computed geometric attention result allows the model to focus on different aspects of local structure using extracted direction and distance features.

At 2109, state information is updated. For example, local state information such as local physical structure information including sequence state information is updated using the geometric attention result determined at 2107. In various embodiments, the updated state information corresponds to the state information received at 2101 updated with the performed geometric reasoning. For example, the geometric attention result can be applied to sequence state information received as local state information and the updated state information can include an updated sequence state with updated weighted sequence representations. In various embodiments, the updated state information can be provided to downstream processing blocks including chained geometric attention blocks.

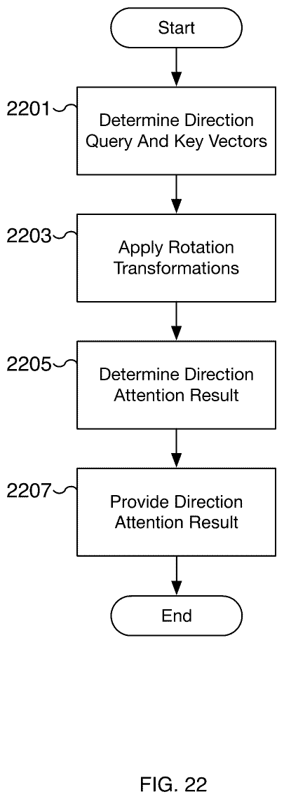

FIG. 22 is a flow chart illustrating an embodiment of a process for determining a direction attention result on biological structure. For example, using the process of FIG. 22, a direction attention block of a geometric attention block can determine direction attention scores from local state and structure inputs. In some embodiments, the direction attention scores are intermediate values that are considered along with distance attention scores to determine weighted attention scores. For example, the direction attention result determined using the process of FIG. 22 can be provided to an attention score block of the geometric attention block to determine weighted attention scores and geometric attention scores. In some embodiments, the process of FIG. 22 is performed at 2103 of FIG. 21 by a direction attention block such as direction attention block 1921 of FIG. 19 and/or direction attention block 2021 of FIG. 20.

At 2201, direction query and direction key vectors are determined. For example, using received local state information including local structure context information, direction query and direction key vectors are determined. In some embodiments, the local state information is sequence state information and corresponds to local state information such as local state information 1901 of FIG. 19 and/or local state information 2001 of FIG. 20. In various embodiments, the direction query and direction key vectors are determined using learned weight matrices such as learned weight query and learned weight direction matrices.

At 2203, rotation transformations are applied. For example, using local physical structure information, the direction query and direction key vectors determined at 2201 are transformed to a shared frame of reference such as a global reference frame. In some embodiments, the applied transformations correspond to performing matrix multiplication operations using rotation matrices of the associated frames. For example, received local physical structure information such as backbone frames can include rotation matrices for transforming the respective elements of direction query and direction key vectors to the appropriate shared frame of reference. In some embodiments, the rotation matrices are derived from the backbone frames information, such as from the coordinate axes and/or origin coordinates of each backbone frame depending on the frame format. In some embodiments, the applied rotation transformations correspond to computational operations 2031 and 2033 of FIG. 20.

At 2205, a direction attention result is determined. For example, the transformed direction query and direction key vectors are compared to determine a direction attention result. In some embodiments, the direction attention result includes multiple direction attention scores by computing a direction attention score between each element and all other corresponding elements in the sequence. For example, a direction attention score for an element can be computed by performing a dot product operation on the corresponding direction query vector and direction key vector elements.

At 2207, the direction attention result is provided. For example, the direction attention result is provided to an attention score block where the direction attention result can be eventually evaluated with a value vector. In some embodiments, the direction attention result can be an intermediate result that is considered along with a distance attention result to determine a weighted attention result. Along with additional attention score processing steps, a normalized weighted attention result can be evaluated with a value vector to determine a geometric attention result. In some embodiments, the attention score block receiving the direction attention result is attention score block 1925 of FIG. 19 and/or attention score block 2025 of FIG. 20.

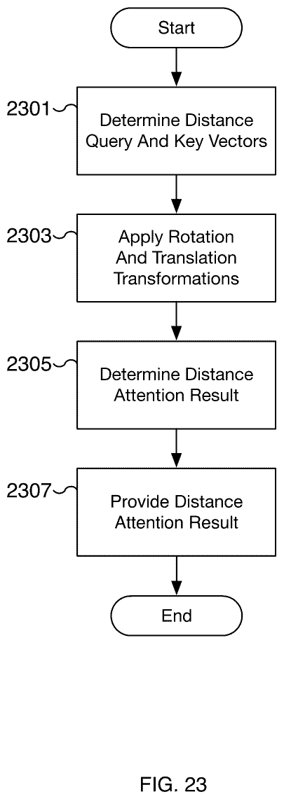

FIG. 23 is a flow chart illustrating an embodiment of a process for determining a distance attention result on biological structure. For example, using the process of FIG. 23, a distance attention block of a geometric attention block can determine distance attention scores from local state and structure inputs. In some embodiments, the distance attention scores are intermediate values that are considered along with direction attention scores to determine weighted attention scores. For example, the distance attention result determined using the process of FIG. 23 can be provided to an attention score block of a geometric attention block to determine weighted attention scores and geometric attention scores. In some embodiments, the process of FIG. 23 is performed at 2105 of FIG. 21 by a distance attention block such as distance attention block 1923 of FIG. 19 and/or distance attention block 2023 of FIG. 20. In various embodiments, the process of FIG. 23 is similar to the process of FIG. 22 but used to perform distance attention scores.

At 2301, distance query and distance key vectors are determined. For example, using received local state information including local structure context information, distance query and distance key vectors are determined. In some embodiments, the local state information is sequence state information and corresponds to local state information such as local state information 1901 of FIG. 19 and/or local state information 2001 of FIG. 20. In various embodiments, the distance query and distance key vectors are determined using learned weight matrices such as learned weight query and learned weight distance matrices.

At 2303, rotation and translation transformations are applied. For example, using local physical structure information, the distance query and distance key vectors determined at 2301 are transformed to a shared frame of reference such as a global reference frame. In some embodiments, the applied transformations correspond to performing matrix multiplication and addition operations using rotation matrices and translation vectors, respectively, of the associated frames. For example, received local physical structure information such as backbone frames can include rotation matrices and translation vectors for transforming the respective elements of a distance query and distance key vectors to the appropriate shared frame of reference. In some embodiments, the rotation matrices and translation vectors are derived from the backbone frames information, such as from the coordinate axes and/or origin coordinates of each backbone frame depending on the frame format. In some embodiments, the applied rotation and translation transformations correspond to computational operations 2041 and 2043 of FIG. 20.

At 2305, a distance attention result is determined. For example, the transformed distance query and distance key vectors are compared to determine a distance attention result. In some embodiments, the distance attention result includes multiple distance attention scores by computing a distance attention score between each element and all other corresponding elements in the sequence. For example, a distance attention score for an element can be computed by performing a Euclidean norm operation with the corresponding distance query vector and distance key vector elements.

At 2307, the distance attention result is provided. For example, the distance attention result is provided to an attention score block where the distance attention result can be eventually evaluated with a value vector. In some embodiments, the distance attention result can be an intermediate result that is considered along with a direction attention result to determine a weighted attention result. Along with additional attention score processing steps, a normalized weighted attention result can be evaluated with a value vector to determine a geometric attention result. In some embodiments, the attention score block receiving the distance attention result is attention score block 1925 of FIG. 19 and/or attention score block 2025 of FIG. 20.

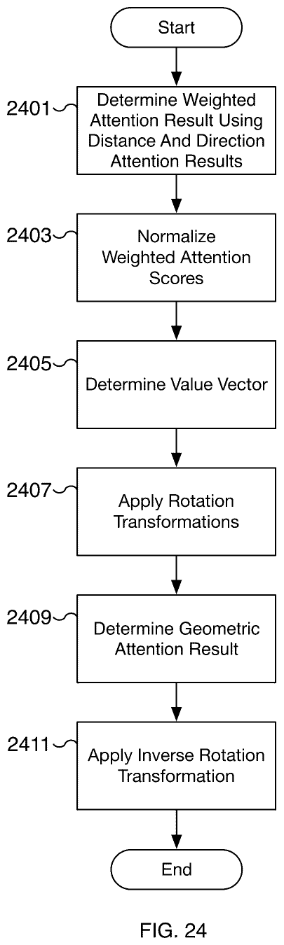

FIG. 24 is a flow chart illustrating an embodiment of a process for determining a geometric attention result on biological structure from direction and distance attention results. For example, using the process of FIG. 24, an attention score block of a geometric attention block can determine geometric attention scores from local state and structure, direction attention, and distance attention inputs. In some embodiments, the geometric attention scores are based on weighted direction attention scores that are attenuated by weighted distance attention scores. For example, the process of FIG. 24 can attenuate the direction attention result determined using the process of FIG. 22 by the distance attention result determined using the process of FIG. 23 prior to applying a value vector to determine a geometric attention result. In some embodiments, the process of FIG. 24 is performed at 2107 of FIG. 21 by an attention score block such as attention score block 1925 of FIG. 19 and/or attention score block 2025 of FIG. 20. In various embodiments, the process of FIG. 24 is combined with at least the processes of FIGS. 22 and 23 to determine a geometric attention result.

At 2401, a weighted attention result is determined using distance and direction attention results. For example, a weighted attention result is determined based on a weighted attention result and a weighted distance result. The intermediate weighted direction attention and intermediate weighted distance attention results can be determined by applying learned and/or configured direction and distance term weights, respectively, to the direction attention and distance attention results. The intermediate weighted distance result scores are then subtracted from the intermediate weighted direction result scores to determine a weighted attention result. For example, for a particular element of a weighted attention result, a weighted attention score for element_{i,j} can be determined with weighted attention result computation 2051 by computing element_{i,j}=weight_direction*direction_term −weight_distance*distance_term. In some embodiments, the operations performed to determine the weighted attention result at 2401 correspond to computational operation 2051 of FIG. 20.

At 2403, weighted attention scores are normalized. For example, the weighted attention result determined at 2401 is normalized into a valid probability distribution. In some embodiments, a softmax function is utilized to normalize the weighted attention scores of the weighted attention result. The normalized weighted attention scores can be evaluated with a value vector. In some embodiments, the normalization operations performed on weighted attention scores correspond to the softmax functional module shown in FIG. 20.

At 2405, a value vector is determined. For example, a value vector is determined using received local state information including local structure context information. In some embodiments, the local state information is sequence state information and corresponds to local state information such as local state information 1901 of FIG. 19 and/or local state information 2001 of FIG. 20. In various embodiments, the value vector is determined using a learned weight matrix such as a learned weight key matrix.

At 2407, rotation transformations are applied. For example, using local physical structure information, the value vector determined at 2405 is transformed to a shared frame of reference as the normalized weighted attention scores determined at 2403. In some embodiments, the applied transformations correspond to performing matrix multiplication operations using rotation matrices of the associated frames. For example, received local physical structure information such as backbone frames can include rotation matrices for transforming the respective elements of the value vector to the appropriate reference frame. In some embodiments, the rotation matrices are derived from the backbone frames information, such as from the coordinate axes and/or origin coordinates of each backbone frame depending on the frame format. In some embodiments, the rotation transformations applied at 2407 correspond to computational operation 2053 of FIG. 20.

At 2409, a geometric attention result is determined. For example, the transformed value vector from step 2407 is evaluated with the normalized weighted attention result from step 2403 to determine a geometric attention result. In some embodiments, the geometric attention result is determined by applying a matrix multiplication operation. In some embodiments, the matrix multiplication operation performed to determine the geometric attention result at 2409 corresponds to computational operation 2055 of FIG. 20.

At 2411, an inverse rotation transformation is applied. For example, an inverse rotation transformation is applied to the geometric attention result determined at 2409 to transform the geometric attention result into the appropriate frame of reference. In various embodiments, the rotation transformation applied at 2411 corresponds to the inverse of the rotation transformation applied at 2407. In some embodiments, the inverse rotation transformation applied at 2411 corresponds to computational operation 2055 of FIG. 20. In various embodiments, the transformed geometric attention result can be used to update the state information.

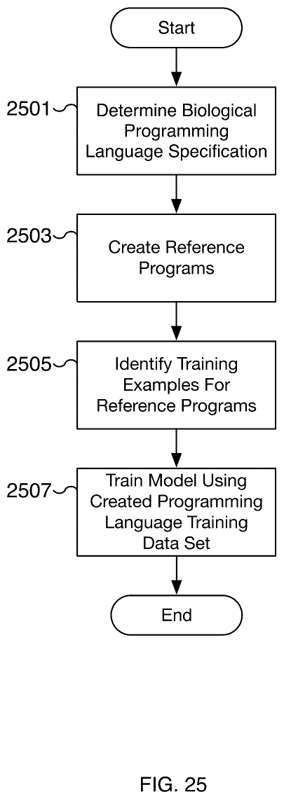

FIG. 25 is a flow chart illustrating an embodiment of a process for training a biological language reasoning model to support a biological programming language. For example, using the process of FIG. 25, a multi-track biological language reasoning model can be trained to support a biological programming language. The biological programming language can be used to create biological language programs that describe conditions for the biological language reasoning model including requirements and constraints. The described conditions are utilized by the biological language reasoning model when predicting biological language reasoning results such as when generating a protein with desired properties. In some embodiments, the process of FIG. 25 is performed at 701 of FIG. 7 by a biological language model service such as biological language model service 111 of FIG. 1 and/or biological language model service 201 of FIG. 2. In some embodiments, the multi-track biological language reasoning model is and/or corresponds to biological language model 303 of FIG. 3, biological language model module 401 of FIG. 4, and/or multi-track biological protein language model 503 of FIG. 5.

At 2501, a biological programming language specification is determined. For example, a biological language program specification is defined that allows a user to express a set of desired conditions for performing biological language reasoning using a biological language reasoning model. In some embodiments, the conditions are associated with one or more keywords including one or more programming language keywords and/or programming language operations. In various embodiments, the determined biological programming language specification allows a user to express the conditioning in a high-level programming language. For example, modular biological language sub-programs can be written and reused as building blocks for creating biological language programs. The biological language programs can further utilize abstract programming concepts and use programming language constructs for describing and simplifying complex biological requirements and constraints.

In some embodiments, the determination of the biological programming language specification includes determining a set of supported conditions and their associated programming language constructs. The supported conditions can be selected for interfacing and controlling a biological language reasoning model such as for the design and generation of biological objects including proteins. Example conditions supported by the biological programming language can relate to functional properties, stability properties, developability properties, immunogenicity properties, symmetries of structure, symmetries of amino acid sequences, structure templates including relative positions of atoms within a subset of residues, portions of a biological object which are surface exposed, secondary structure on portions of proteins, hydrophobic amino acid properties of proteins including the quantity of hydrophobic amino acids, the globularity of a portion of a protein, functional specificity for a portion of a biological object, molecular interactions and interfaces including protein to protein interactions and/or interfaces, small molecular interactions, deoxyribonucleic acid (DNA)/ribonucleic acid (RNA) interactions and binding properties, motif and active site scaffolding, and/or post translational modifications, among other conditions. In various embodiments, the biological programming language specification provides a standardized approach for specifying the conditions and each of the conditions can have one or more corresponding program language keywords for describing the context associated with the condition.

In various embodiments, the biological programming language specification can significantly expand the accessible functionality and usefulness of the biological language reasoning model. For example, the biological programming language specification can support describing structure templates, surface exposed atoms, secondary structure, functional specificity on both global and per-residue granularity, etc. Moreover, the biological programming language specification can be expanded over time and the model trained to support the newly supported conditions.

At 2503, reference biological language programs are created. For example, a set of reference biological language programs that utilize the conditions defined by the biological programming language specification determined at 2501 is created. In various embodiments, the reference biological language programs are example biological language programs written to conform to the biological programming language specification and that utilize the different conditions supported by the biological programming language specification. When combined with corresponding example results that align with the objectives of the created reference biological language programs, the created reference biological language programs are used to train the biological language reasoning model. In some embodiments, for each supported condition and/or for a given portion of the biological programming language specification, a reference biological language program is created.

At 2505, training examples are identified for the reference biological language programs. For example, training data examples for the supported biological programming language conditions expressed by the reference biological language programs created at 2503 are identified. In various embodiments, the training examples are identified for creating a training data set for training a biological language reasoning model to support the conditions defined by the biological programming language specification determined at 2501. For example, for each supported condition and/or for a given portion of the biological programming language specification, a reference biological language program created at 2503 is matched with corresponding examples of biological objects such as proteins exhibiting the described condition(s). The identified training examples can be used as target results associated with the reference biological language programs and, along with the reference programs, are used to create a training data set. The identified examples, target results, and/or training data can be created by mining data sources of natural proteins such as public and/or private data stores and/or by utilizing synthetic data.

In some embodiments, the training examples can be expanded with synthetic data including by utilizing synthetic data to augment other data and/or as the primary source of training data. For example, the biological language reasoning model can be initially trained using natural or experimental data such as natural protein examples mined from natural proteins. Synthetic data can be created and used to augment the natural or experimental data. In some embodiments, the training is performed entirely with synthetic data, for example, in the event natural or experimental data is not readily available. In various embodiments, the most recent model is then used to generate biological objects using a biological language program. Generation results that do not properly match the biological language program can be rejected and the remaining generation results, such as the results that align with the biological language program, can be included in the training data set. Using the revised training data augmented with synthetic data, the model can be retrained for improved performance. This process can be repeated, potentially continuously, to increase the size and/or quality of the training data and the resulting trained biological language reasoning model.

In various embodiments, each supported condition, which is specified in accordance with the determined biological programming language specification, may require a specialized approach for identifying training examples. In the following sections, the context related to specific conditions is described and can include details such as how a condition is expressed using a biological programming language (such as by a reference biological language program used during training or another biological language program used during inference), how training examples meeting the condition are identified for creating a training data set, and how the model is eventually prompted to support the condition. The following disclosures are also provided as examples for how to extend the biological programming language specification to support additional conditions and are included to provide one of skill in the art direction on how to expand the biological programming language specification to support a new condition including how to create a biological language program utilizing the new condition, how to train the biological language reasoning model to understand the new condition, and/or how to prompt the biological language reasoning model with respect to the new condition.

In some embodiments, a condition is supported by the biological language reasoning model using one or more conditioning vectors or tensors and/or the condition is specified using one or a variety of approaches supported by the determined biological programming language specification. In certain embodiments, examples of a condition are identified by calculating one or more metrics on candidates to identify which candidates meet the condition. Similarly, certain existing approaches may be available including existing third-party tools or data sources for identifying training examples from candidates. In various embodiments, the training data set may be used to create a conditioning track for the biological language reasoning model. The conditioning track and corresponding conditioning input may be integrated with existing tracks of the model, such as tracks related to structure, function, or amino acid sequence (among other available tracks) and/or the conditioning track may exist as its own conditioning input track for the biological language reasoning model.

In some embodiments, conditions related to symmetries of structure can include identifying training examples of symmetries in protein structures. In some embodiments, symmetries of structure are identified by finding protein complexes with repeated chains (e.g., AA, ABA, etc.). In some embodiments, atomic coordinates are clustered to identify key structural motifs. The structural motifs are then evaluated for symmetries. In some embodiments, dynamic programming is utilized to break a biological object such as a protein into different chunks including chunks exhibiting symmetry. In some embodiments, synthetic symmetric data is constructed by constraining the sequence that is repeated and/or by sampling using an energy-based model. In various embodiments, for each of the positions appropriately associated with symmetry, the structure correspondences and/or groupings are marked within an array and associated with a conditioning vector. For example, an N×N array can be created with the distances from the centers-of-mass and/or other forms of adjacencies between symmetric chunks of the proteins. In various embodiments, the created array corresponds to a conditioning vector for the arrangement of symmetries.

In some embodiments, conditions related to symmetries of an amino acid sequence can include identifying repeated patterns or motifs within the sequence. For instance, dynamic programming for local sequence alignment can be used to identify repeated and/or symmetrical sequences, for example, by applying a Smith-Waterman algorithm. In various embodiments, for each of the positions appropriately associated with symmetries of an amino acid, the structure correspondences and/or groupings are marked within an array and associated with a conditioning vector.

In some embodiments, conditions related to the globularity of a protein or a portion of a protein can include identifying training examples by computing globularity from a corresponding structure. The globularity can be computed by measuring the radius of gyration (e.g., the mass-weighted distribution of atoms around a protein's center of mass). In various embodiments, the model can be conditioned on a single scalar value. In some embodiments, the model is conditioned on a quantized tensor such as a one-hot tensor.

In some embodiments, conditions related to structure templates can include identifying relative positions of atoms within a subset of residues. Structure template related conditions can be implemented using frame conditioning during a pretraining stage for the biological language reasoning model.

In some embodiments, conditions related to portions of a protein which are surface accessible and/or exposed can include identifying training examples by computationally scoring which residues in a protein sequence/structure are surface exposed. In some embodiments, the scoring is performed using a Shrake-Rupley algorithm based on a rolling probe approach. For example, for each candidate protein in a dataset with experimentally characterized structures or computationally estimated structures, the protein is scored to determine a set of scores for each atom being exposed to solvent. The atom scores for each residue are combined into a residue score, for example, by computing a mean value. Other summary statistics are appropriate as well. A vector of scores, such as a vector of floating-point scores, can be created that corresponds to the length of the protein sequence. The generated vector can be used to report the surface exposed, the surface not exposed, and/or where data is missing. In some embodiments, the calculated scores are compared to threshold values to determine exposure results. In some embodiments, the generated vector is included as a conditioning track for training the biological language reasoning model, and a corresponding output track is also included for the model. In various embodiments, the model can be configured to be trained using a MaskGIT cross-entropy objective thereby allowing the model to be responsive to these inputs.

In some embodiments, conditions related to the secondary structure of portions of proteins can include identifying training examples by computationally scoring the secondary structure of a protein sequence and/or protein structure. The computational result can be in the format of a classification result of the secondary structure (e.g., a beta sheet, an alpha helix, a coil, etc.). For example, for each candidate protein in a dataset with experimentally characterized structures or computationally estimated structures, the protein is analyzed to determine a classification result for each residue. A vector of classification results can be created that corresponds to the length of the protein sequence. In some embodiments, the generated vector is included as a conditioning track for training the biological language reasoning model, and a corresponding output track is also included for the model. In various embodiments, the model can be configured to be trained using a MaskGIT cross-entropy objective thereby allowing the model to be responsive to these inputs.

In some embodiments, conditions related to hydrophobic amino acid properties of proteins including the quantity of hydrophobic amino acids and whether a portion of proteins should have many or few hydrophobic amino acids can include identifying training examples by computing the proportion (or count or another appropriate metric) of amino acids which are hydrophobic in a protein or in a portion of a protein. In some embodiments, a conditioning vector is created that specifies whether there are many or few amino acids which are hydrophobic around a residue position.

In some embodiments, conditions related to functional specificity for a portion of a biological object such as a protein can include identifying training examples using existing computational tools including tools that provide a functional analysis of proteins by classifying them into families and predicting domains and important sites. The computational tools can provide functional annotation estimation which can be used by a functional tokenizer. With a large vocabulary of function labels and corresponding amino acid sequence positions or residues within a protein, the function labels at each sequence position can be converted into a single vector representing the saliency of text keywords that describe the function in plain text descriptions. In some embodiments, the single vector is compressed and tokenized, for example, using locality sensitive hashing (LSH). In some embodiments, the model is prompted directly with a multi-hot matrix of functional labels.

In some embodiments, conditions related to taxonomy can include identifying training examples from existing taxonomy information. In addition, a taxonomy classifier that determines a taxonomy label from an animo acid sequence input can be trained to generate taxonomy information for sequences that lack taxonomy information. In some embodiments, a vocabulary of all nodes in the tree is created and a corresponding multi-hot vector can be used to provide the taxonomy information to the model. In some embodiments, the taxonomy tree is encoded using one or more graph encoding algorithms and/or neural networks.

In some embodiments, conditions related to electrostatics can include identifying training examples using existing approaches for estimating the electrostatic field for proteins. An electrostatic field estimation can be converted into a prompt by training an autoencoder to compress the local electrostatic field around an amino acid. In some embodiments, latents or tokens are used to represent local electrostatic fields. In various embodiments, the learned latents and/or tokens are generated using approaches similar to those used to compress local structure into structure tokens.

In some embodiments, conditions related to binding interfaces and/or protein to protein interactions can include identifying training examples by mining a dataset of multimers and identifying positions in contact. In some embodiments, an array is created with zeros for positions not in contact (or not on the interface) and ones for positions in contact (or on the interface). In some embodiments, binding interfaces can be provided as input into the model either as a binary signal indicating which amino acids participate in a binding interface, or as a pairwise feature indicating which sequence positions interact with each other.

In some embodiments, conditions related to small molecule interactions can include identifying which small molecule is involved in the interaction and whether/how the interaction occurs. In various embodiments, different approaches are used to indicate which small molecule is involved in the interaction. For example, the biological language reasoning model can be prompted using a compatible chemical format or notation such as a line notion format. In some embodiments, the line notion format corresponds to the simplified molecular-input line-entry system (SMILE) specification. As additional examples, the model can be prompted with a 2D bond graph, and/or, if the structure of the small molecule is available, the model can be prompted with the appropriate atomic coordinates. With respect to indicating how and/or where the interaction occurs, in certain scenarios this is unknown, and the model determines the binding location. In the event it is known which amino acids are involved in the interaction, the model can be prompted with a Boolean tensor indicating the positions of interaction. In some scenarios, the atomic coordinates of the binding pocket are known, and the model can be prompted with the coordinates of the small molecule along with the coordinates of the backbone and/or sidechain atoms of the local protein structure around the binding pocket. In various embodiments, the provided coordinates are provided in the same global reference frame.

In some embodiments, conditions related to deoxyribonucleic acid (DNA)/ribonucleic acid (RNA) interactions and binding properties can be prompted similar to the approach used for small molecule interactions. For example, DNA/RNA interactions and binding can be prompted by identifying which molecule is involved in the interaction and whether/how the interaction occurs. In the context of DNA/RNA, the identity of the molecule can be defined by a sequence rather than via a SMILES string or a 2D bond graph. For example, to indicate which deoxyribonucleic acid (DNA) or ribonucleic acid (RNA) molecule is interacting, the model can be prompted with the appropriate DNA or RNA sequence. In the event the relevant nucleic acids fold into 3D structures and the 3D structures are known, the model can be further prompted with atomic coordinates. Similarly, with respect to indicating how and/or where the interaction occurs, the approach used can be similar as the approach used for small molecule interactions. For example, in certain scenarios, how and/or where the interaction occurs is unknown, and the model determines the binding location. In the event it is known which amino acids are involved in the interaction, the model can be prompted with a Boolean tensor indicating the positions of interaction. In some embodiments, the model is prompted with a pairwise Boolean matrix indicating which DNA or RNA positions interact with which amino acids in the protein. In some scenarios, the atomic coordinates of the binding pocket are known, and the model can be prompted with the coordinates of the DNA/RNA along with the coordinates of the backbone and/or sidechain atoms of the local protein structure around the binding pocket. In various embodiments, the provided coordinates are provided in the same global reference frame.

In some embodiments, conditions related to motif and active site scaffolding can include identifying training data by randomly choosing motifs or coordinating residues from protein structures to act as active sites. The biological language reasoning model can be prompted by indicating the coordinates and amino acid identities of the motif/active site residues.

In some embodiments, conditions related to post translational modifications can include developing a vocabulary of post translational modifications and indicating which sites of the selected training examples undergo post translational modifications. The selected sites can be described via metadata information. In various embodiments, a post translational vocabulary may include entries to support different post translational modifications including entries corresponding to phosphorylation, ubiquitination, acetylation, methylation, glycosylation, and sulfation, among others. The biological language reasoning model can be prompted to design proteins for post translational modification by learning embeddings for each of the supported modification types and adding the learned embeddings position-wise where modifications should occur.

In some embodiments, conditions related to language model derived keywords (including keywords for a protein) can include deriving the keywords using large language models (LLMs) including available and third-party LLMs trained for text summarization. The selected LLMs can be instructed to summarize keywords from text entries sourced from biological data sources such as UniProt. Additional sources such as relevant journal articles and publications can be used in the LLM context and/or used as additional sources for LLM derived keywords. In various embodiments, the text corresponding to the language derived keywords can be used as global conditioning to the biological language reasoning model. For example, a learned text embedding mechanism similar to the disclosed function tokens supported by the biological language reasoning model can be applied for language model derived keywords.

In some embodiments, conditions related to language model derived latents (including latents for a protein) can include deriving the latents using large language models (LLMs). For example, biological source data such as UniProt text entries, journal articles, and publications can be embedded with respect to selected LLMs. The selected LLMs can also be finetuned on biological text for improved representation learning performance. In some embodiments, an LLM is fine-tuned to align a latent space representation with the representations of sequence and/or structure used by the biological language reasoning model. At inference time, the biological language reasoning model can then be prompted with any of text, protein sequence, or protein structure and the provided input would be in the same shared functional latent space. In various embodiments, the approach can utilize a contractive loss to align the latent space representations. In some embodiments, the embeddings can be used as global conditioning to the biological language reasoning model.

In some embodiments, conditions related to language model derived symbols (including symbols for a protein) can include deriving the symbols using large language models (LLMs). In various embodiments, the symbols can be derived using an LLM from biological source data including existing databases, journal articles, and publications. The symbols can be derived similar to the approach taken with respect to language model derived keywords and language model derived latents. The corresponding embeddings can be used as global conditioning to the biological language reasoning model.

In some embodiments, conditions related to adjacency conditioning allow specifying which portions of a biological object such as a protein should be close to one another and/or in contact with one another. The adjacency conditioning conditions can include creating a matrix (such as a symmetric L×L matrix, where L is the length of the protein) where there are zeros or ones based on adjacency properties. For example, when the residues are greater than a configured distance from one another, the corresponding matrix value can be set to zero. Alternatively, when residues are within the configured distance from one another, the corresponding matrix value can be set to one. In various embodiments, the biological language reasoning model is conditioned on the adjacency properties matrix during training.

In some embodiments, an L×L adjacency properties matrix can be condensed into a coarser representation. For example, the matrix can be condensed based on continuous runs of secondary structure. As one example, in a scenario where the secondary structure changes K times, the condensed adjacency properties matrix would have dimensions (K+1)×(K+1), which is smaller than an L×L adjacency properties matrix. In various embodiments, the values of the matrix elements are set to one or zero based on either a minimum distance between any residues in the two groupings or based on the average distance between the residues in the groupings. In some embodiments, these approaches are combined with secondary structure prompting and the size of each group to create a novel interface. For example, a model can use secondary structure (such as with entries on the diagonal), the (K+1)×(K+1) adjacency properties matrix, and the length of each of the secondary structure groups (such as corresponding to the right, Y-axis) to prompt the model to generate a particular type of protein. In some embodiments, the disclosed programming language conditioning is used to create the corresponding interface.

In some embodiments, conditions related to the free energy of the protein can include identifying training examples by estimating the free energy of candidates. The free energy can be estimated by calculating an energy score including by approximating Gibbs free energy such as by using an all-atom energy function that includes terms for Van der Waals interactions, electrostatics, solvation, and hydrogen bonding. In various embodiments, the free energy can be determined for candidate proteins, such as those sourced from a database of proteins, by applying the appropriate computational oracles. The biological language reasoning model can be conditioned on the determined results such as the determined scalar results or a version of the determined scalar results such as a one-hot quantized version generated from the determined scalar results.

In some embodiments, conditions related to pH, salinity, and/or temperature growth conditions can include identifying training examples using associated organism metadata included with existing sequence data. For example, many of the organisms referenced in the available metadata have known growth conditions and/or growth conditions that can be estimated. In various embodiments, an organism growth conditions predictor can be used to assign each sequence in the training data set a pH, salinity, and temperature value. The corresponding values can be tokenized, for example, by binning the values. In various embodiments, the generated tokens can be provided to the biological language reasoning model.

In some embodiments, conditions related to Multiple Sequence Alignment (MSA) can include deriving and constructing MSA sequences. In various embodiments, MSAs can be used to determine information about coevolutionary statistics of proteins, which can be used to improve tasks such as structure prediction and generating proteins from a given protein family. In some embodiments, MSAs are derived for sequences by querying databases of reference sequences and/or searching for (sub)sequences with strong alignment to the query. Once an MSA is constructed, the set of sequences is tokenized. For example, the set of sequences can be tokenized by applying the tokenization approach used to tokenize the primary amino acid sequence. In some embodiments, once the set of sequences is tokenized, a (sequence length×MSA depth) matrix is encoded for processing in the biological language reasoning model by using an MSA encoder with axial attention. In some embodiments, an MSA transformer is used that allows for the efficient encoding of MSAs.

At 2507, the biological language reasoning model is trained using the created programming language training data set. For example, using the training data set generated from the biological language programs created at 2503 and the matching training examples identified at 2505, the biological language reasoning model is trained to support the biological programming language specification determined and defined at 2501. In various embodiments, the model is trained using the reference biological language programs as conditioning inputs and their corresponding identified training examples as outputs. As described in step 2505, the trained model can be used to generate additional synthetic training data for retraining the model for improved performance and/or support for biological language programs. In some embodiments, the biological language reasoning model is trained and/or additionally conditioned based on the conditioning configuration for the specific supported condition. The conditioning configuration can include the application of a specific conditioning vector and/or specific conditioning parameters described at 2505.

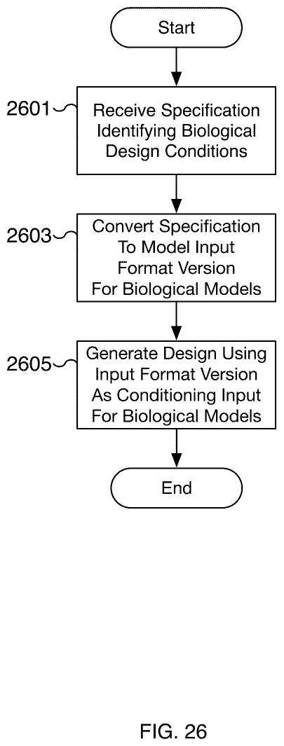

FIG. 26 is a flow chart illustrating an embodiment of a process for converting biological design conditions to conditioning input for a biological reasoning model using a biological programming language. For example, using the process of FIG. 26, a biological programming specification conforming to a biological programming language is used to express biological design goals such as design conditions and constraints. The biological programming specification corresponds to a biological program that can initiate the generation of candidate designs using one or more biological reasoning models. In some embodiments, the compilation process is performed using a machine learning model, such as a large language model (LLM). In some embodiments, the process of FIG. 26 is performed at 703 and/or 705 of FIG. 7 by a biological language model service such as biological language model service 111 of FIG. 1 and/or biological language model service 201 of FIG. 2. In some embodiments, one of the targeted biological reasoning models is a multi-track biological language reasoning model and corresponds to biological language model 303 of FIG. 3, biological language model module 401 of FIG. 4, and/or multi-track biological protein language model 503 of FIG. 5. In some embodiments, the model is at least partially trained using the process of FIG. 25.

At 2601, a biological programming specification identifying biological design conditions is received. The received biological programming specification conforms to a biological programming language designed to express complex biological requirements. For example, using a high-level language, a biological programming specification may define one or more design goals, such as secondary structure constraints (e.g., an alpha helix between residues 10-30), surface exposure requirements, binding interfaces, symmetry constraints, functional properties, functional motifs, and/or stability properties, among others. Additional design goals and constraints may include properties related to developability and immunogenicity. For example, using the disclosed techniques and platforms, design goals can specify developability and immunogenicity characteristics of a candidate protein, particularly in the context of protein therapeutics. In some embodiments, the design goals can include matching a target embedding, such as to achieve desired functional properties of the corresponding target object. For example, the embeddings generated under a biological reasoning model are selected based on matching a target embedding with the desired functional properties.

In various embodiments, the received biological programming specification may be written directly by a user through a text-based interface and/or generated via a graphical interface that maps user selections into the biological programming language format. In some embodiments, the user interface utilizes a natural language interface, and the biological design requirements are composed in a natural language format. For example, the biological design requirements can be provided via a natural language interface, such as through a natural language interface of a large language model (LLM). Moreover, the user can have a back-and-forth conversation with an LLM to automatically generate and refine the biological programming specification. In some embodiments, the conversion is performed by a natural language processing agent, such as an artificial intelligence (AI) agent, including by an LLM-based agent. In some embodiments, the biological programming specification is generated using an AI-enhanced development environment, such as a programming or development environment enhanced with one or more AI agents for generating and refining the biological programming specification and its specified conditions, such as the requirements and constraints specified in the biological programming specification. For example, an LLM-based AI agent can be utilized to help generate the biological programming specification, including to validate the accuracy, performance, and feasibility of the specification.

In various embodiments, the biological programming language supports composability and modularity, allowing multiple conditions to be combined into a single biological program. Using the biological programming specification, users can define highly specific and/or multi-objective protein design tasks. In various embodiments, the biological programming specification forms the starting point for the compilation process and serves as a description of the target design intent for subsequent model input generation.

At 2603, the biological programming specification is converted to a model input format version for one or more biological reasoning models. For example, the biological programming specification can be compiled or translated to target one or more different biological reasoning models, such as a protein folding model, a diffusion-based model, a text diffusion LLM, an autoregressive decoder LLM, a structure-based foundation model, and/or a multi-track transformer-based language model, among others. In some embodiments, the conversion is performed using a machine learning model, such as a large language model (LLM)-based compiler that interprets the structure and semantics of the biological program and translates it into model-compatible inputs. In various embodiments, the conversion may first generate a model-neutral intermediate representation. The intermediate representation captures the full set of biological constraints and design requirements, and may utilize a syntax tree, structured object graph, or another representative data structure. The model-neutral intermediate representation may then be used to generate the appropriate conditioning inputs required by the targeted biological model(s). Each targeted model may, depending on its architecture, utilize a different version of an input format. For example, one model may require per-residue token tracks for structural constraints, while another may use span-based conditioning or global embedding tensors for functional context. At 2603, the conversion process ensures that each target model receives inputs for conditioning in the format and structure it requires.

At 2605, a biological design is generated using the input format version as conditioning input for one or more biological reasoning models. The conditioning input, generated during the compilation and conversion process, guides the generative behavior of each targeted model to produce candidate biological outputs that satisfy the specified constraints and requirements. For example, the targeted models receive the conditioning input and perform one or more inference passes to generate and predict a biological design, such as a protein sequence and/or structure that aligns with the design goals specified in the biological programming specification. In some embodiments, multiple models are targeted to perform the generation of the biological design in a collaborative, parallel, and/or adversarial manner. For instance, two or more models may each operate on their own conditioning inputs to independently propose candidate designs, which can then be evaluated, compared, and/or merged. In some embodiments, the design workflows are collaborative, and different models may refine the output of other models, such as one generating a protein structure and another optimizing the protein sequence. The generation workflow can further apply an iterative joint optimization across two or more design axes, such as structure, sequence, and function. As another example, structurally focused models can be used to validate and/or filter outputs generated by function-oriented models, enhancing the robustness and biological plausibility of the final designs. In some embodiments, the candidate designs can be selected by optimizing residue embeddings, as determined by a biological reasoning model, to closely match a target embedding. For example, embeddings generated by an LLM or another biological reasoning model may be aligned or made to closely correlate with a target embedding as one technique to achieve specific design objectives, such as desired functional properties.

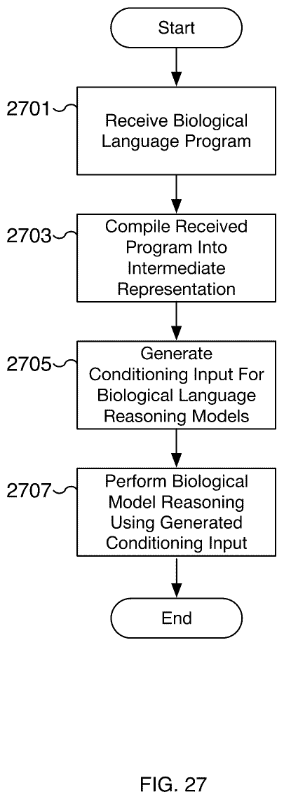

FIG. 27 is a flow chart illustrating an embodiment of a process for interfacing with a biological language reasoning model using a biological language program. For example, using the process of FIG. 27, a biological language reasoning model, such as a multi-track model or another type of biological model, can perform biological reasoning in response to a biological language program. The biological language program can specify conditions such as protein design and generation constraints and requirements that are used as inputs to the biological language reasoning model. In various embodiments, the biological language program is compiled into conditioning input, such as one or more input conditioning tracks, for the biological language reasoning model. Other input formats or input format versions are appropriate as well. In some embodiments, the process of FIG. 27 is performed at 703 and/or 705 of FIG. 7 by a biological language model service such as biological language model service 111 of FIG. 1 and/or biological language model service 201 of FIG. 2. In some embodiments, the process of FIG. 27 is performed as part of the process of performing the process of FIG. 26. In some embodiments, one of the targeted biological reasoning models is a multi-track biological language reasoning model and corresponds to biological language model 303 of FIG. 3, biological language model module 401 of FIG. 4, and/or multi-track biological protein language model 503 of FIG. 5. In some embodiments, the model is at least partially trained using the process of FIG. 25.

At 2701, a biological language program is received. In various embodiments, the received biological language program describes one or more conditions including requirements and/or constraints for performing biological language reasoning using a biological language reasoning model. For example, the biological language program can describe requirements for generating candidate binder proteins to bind to a binding target protein. As another example, the biological language program can specify required protein to protein interactions and/or interfaces or deoxyribonucleic acid (DNA)/ribonucleic acid (RNA) interactions and/or bindings. In some embodiments, the biological language program specifies one or more target biological reasoning models, such as a protein folding model and/or a multi-track biological language reasoning model. Other target models are appropriate as well, such as such as a diffusion-based model, a text diffusion LLM, an autoregressive decoder LLM, and/or a structure-based foundation model, among others. In various embodiments, the received biological language program is written to conform to a biological language program specification supported by the biological language reasoning model.

At 2703, the received biological language program is compiled into an intermediate representation. For example, the biological language program received at 2701 is compiled into an intermediate representation such as a syntax tree. In some embodiments, the intermediate representation corresponds to a simplified version of the biological language program and may be a normalized and/or model-agnostic compiled version of the received biological language program. The intermediate representation may further be encoded in a manner that can be visualized. For example, a syntax tree representing the biological language program can include non-terminal and terminal nodes. The different nodes can be associated with different conditions specified by the biological language program. For example, non-terminal nodes can require that the associated child nodes are symmetric. As another example, a terminal node can require a certain length constraint. Additional constraints, as disclosed herein above, can be supported as well. In some embodiments, the syntax tree can be provided in a visual format that allows a user to visualize and/or modify the conditions described by the biological language program. In some embodiments, the compilation step performed at 2703 is performed using a large language model (LLM). For example, an LLM can be prompted to generate an intermediate representation from the received biological language program.

At 2705, a conditioning input is generated for the target biological language reasoning models. For example, one or more conditioning tracks and/or tensors or other forms of conditioning input that conform to the biological language program received at 2701 are generated for the target biological language reasoning model(s). In various embodiments, the conditioning input is generated from the intermediate representation created at 2703 and/or as part of the compilation process performed at 2703. For example, as part of or as a continuation of the compilation process started at 2703, the intermediate representation generated at 2703 can be converted into conditioning input for one or more input conditioning tracks of the biological language reasoning model. In some embodiments, conditioning input is generated for multiple different input tracks for the biological language reasoning model. For example, structure conditioning constraints may be generated for a structure track of the model and function conditioning constraints may be generated for a function track of the model. In some embodiments, the biological language reasoning model utilizes a single biological programming language conditioning track, and the biological language program is compiled into a single conditioning input track that represents the set of conditions including constraints and/or requirements described by the biological language program. In various embodiments, the conditioning input is formatted to comply with the input format requirements of the target biological reasoning model(s). In some embodiments, multiple models are targeted, and multiple different conditioning inputs are generated, each for the appropriate target model.

In some embodiments, the conditioning input generated at 2705 is produced using a large language model (LLM). For example, as part of the compilation process, an LLM can be prompted to generate the conditioning input for a target model from an intermediate representation of the biological language program. In certain embodiments, the LLM can dynamically translate the intermediate representation into conditioning inputs suitable for the target biological reasoning model(s). In various embodiments, the LLM is prompted with details of the biological programming language, documentation on the intermediate representation, example programs, and corresponding example input and/or input format requirements for each target biological reasoning model, among other contextual details. Based on the target biological reasoning model (e.g., a protein folding model, a diffusion-based model, a text diffusion LLM, an autoregressive decoder LLM, a structure-based foundation model, and/or a multi-track biological language reasoning model, among others), the LLM converts the intermediate representation into model-specific conditioning inputs. For example, different model input formats may require a per-residue token input format, span-level annotations, global conditioning vectors or tensors, and/or geometry-based embeddings, among others.

At 2707, biological model reasoning is performed using the generated conditioning input. For example, using the conditioning input generated at 2705, biological model reasoning is performed by the targeted biological language reasoning model(s). In some embodiments, each target model receives additional input in addition to the conditioning input determined by the biological language program. In various embodiments, the biological model reasoning performed corresponds to a biological language query, and one or more inference passes of a biological language model are performed to refine the search query results. In various embodiments, the biological model reasoning is performed by applying multiple transformer encoder layers including multiple attention mechanisms of a biological language foundation model. The attention mechanisms can include self-attention and/or geometric attention mechanisms based on the biological language learned during training. In some embodiments, additional post-processing such as fine tuning can be performed to refine the prediction results including to implement certain conditions described by the biological language program.

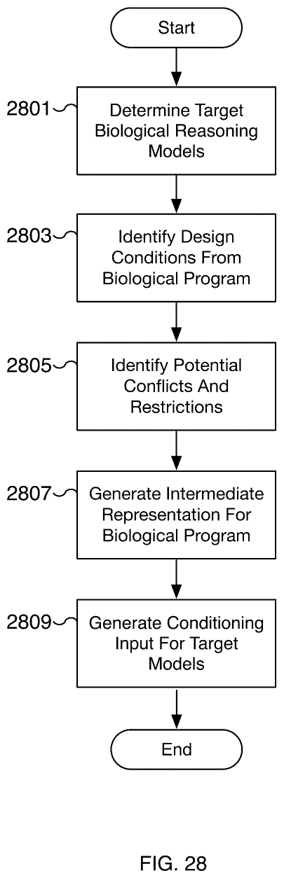

FIG. 28 is a flow chart illustrating an embodiment of a process for compiling a biological programming specification into model-compatible conditioning inputs. Using the process of FIG. 28, a compiler, including an LLM-based compiler, is used to translate a high-level biological programming specification describing a biological design to conditioning inputs for one or more targeted biological reasoning models. In various embodiments, the generated conditioning inputs conform to the input format requirements of the one or more targeted biological reasoning models. In some embodiments, the compilation process utilizes a model-neutral intermediate representation that is compatible with visualization tools. For example, the biological programming specification is compiled into an intermediate representation, which is then converted to the format required by a targeted model. In some embodiments, the process of FIG. 28 is performed at 2601 and/or 2603 of FIG. 26 and/or at 2701, 2703, and/or 2705 of FIG. 27 in response to a received biological programming specification targeting one or more biological reasoning models. In some embodiments, the process of FIG. 28 is performed at 703 and/or 705 of FIG. 7 by a biological language model service such as biological language model service 111 of FIG. 1 and/or biological language model service 201 of FIG. 2. In some embodiments, one of the targeted biological reasoning models is a multi-track biological language reasoning model and corresponds to biological language model 303 of FIG. 3, biological language model module 401 of FIG. 4, and/or multi-track biological protein language model 503 of FIG. 5.

At 2801, target biological reasoning models are determined. For example, one or more target biological reasoning models are determined for the application of the biological programming specification. The selected models may include a variety of one or more different models, for example, a protein folding model for generating three-dimensional structures, a transformer-based sequence generative model for optimizing amino acid sequences, or a multi-track biological reasoning model that integrates multiple tracks simultaneously, such as tracks based on sequence, structure, and function constraints, among others. In various embodiments, the target models are determined based on requirements specified by the received biological programming specification and/or the models are specified directly in the biological programming specification. For example, the target models can be selected automatically based on the types of constraints, conditions, and/or objectives defined in the biological program. For instance, in the event the biological programming specification includes surface accessibility and binding site constraints, a model capable of interpreting spatial geometry and binding affinity predictions can be automatically selected. In some embodiments, users may manually select and/or override the target model list, enabling the selection of different inference strategies and/or the evaluation and application of outputs across different architectures and models.

At 2803, design conditions from the biological program are identified. In various embodiments, the conditions represent the biological design goals, constraints, and/or requirements for generating a target biological object, such as a target protein. For example, the biological program or biological programming specification may specify that a protein should contain an alpha helix in a specific residue range, exhibit particular physical symmetry, include a desired binding motif, and/or be surface-accessible in defined regions, among other conditions. In various embodiments, the compiler extracts and identifies these design conditions, which may include protein design conditions, for generation of an intermediate representation and for conversion to a supporting input format of a target biological reasoning model. In some embodiments, the identified conditions are categorized, such as by type and/or properties, such as by structural and functional properties. In some embodiments, conditions may also include high-level objectives like stability, thermostability, immunogenicity avoidance, and/or binding affinity thresholds. Additional conditions may include functional, developability, and immunogenicity properties of a candidate protein, particularly in the context of protein therapeutics. In various embodiments, the identification step is performed to ensure that all specified and/or required design goals are represented and can be used to identify missing or incompatible design conditions at subsequent compilation steps.

At 2805, potential conflicts and restrictions are identified. For example, the design conditions identified at 2803 are analyzed to identify potential conflicts and restrictions, such as invalid conditions, incompatible conditions, inconsistent conditions, missing conditions, infeasible requirements, impractical requirements, and/or potentially restricted design goals, among other potential downstream errors. For example, the specification may include design requirements for a highly flexible but also rigid structure, which may be physically or functionally unrealistic. As another example, the specification may specify two binding motifs that overlap in sequence or spatial placement. In various embodiments, the compilation process evaluates the specified conditions for mutual exclusivity, redundancy, and compliance with physical, biological, safety, or ethical constraints such as spatial limits, motif embedding tolerances, solvent accessibility thresholds, and/or harmful or prohibited functionality, among others. In some embodiments, the identified issues can be logged, presented to a user for review, provided with automatically proposed revisions, and/or automatically revised such as by relaxing constraints, prioritizing constraints, sanitizing conditions, and/or revising conflicting conditions. In some embodiments, the identification step is a screening layer for implementing ethical and security safeguards. For example, the compilation step can detect and flag high-risk constraints, such as functionality associated with pathogenic proteins, neurotoxins, or viral entry domains, and notify the user of and/or prevent the generation of potentially harmful or unauthorized biological outputs. In various embodiments, the validation step further ensures that the biological program is compatible with the target model and its limitations, and that the design goals can be reliably compiled as conditioning input for a selected target biological reasoning model. Moreover, the early identification of potential conflicts and restrictions at step 2805 can avoid expending expensive resources, such as compute and time, on downstream tasks such as inference passes or experimental synthesis of candidate designs in wet laboratories for design candidates that are unlikely to achieve the desired design goals.

At 2807, an intermediate representation is generated for the biological program. The generated intermediate representation can function as a model-neutral abstraction of the biological design conditions identified at step 2803 in the compilation process. In various embodiments, the intermediate representation captures the structure, hierarchy, and semantics of the input biological language specification in a format that can be more easily visualized, validated, and transformed into model-specific input. For example, the intermediate representation may take the form of a syntax tree, a structured object graph, a prioritized list of normalized conditions, and/or a table of normalized conditions. Moreover, the intermediate representation can be used to annotate different regions of a proposed protein sequence and/or structure. For example, the intermediate representation may include representations or references to the design conditions identified at 2803, such as by associating each identified condition with a corresponding node of a syntax tree or object graph. In some embodiments, the syntax tree of an intermediate representation includes both terminal and non-terminal nodes, and a specific condition of the identified conditions associated with a non-terminal node of the syntax tree applies to each of the child nodes of the non-terminal node.

In some embodiments, the intermediate representation generated at 2807 includes annotations for structural motifs, residue-level constraints, symmetry groupings, and/or contact locations. The intermediate representation enables downstream steps, including an LLM-based compiler, to operate on a consistent and reusable data structure independent of the specific architecture or input format requirements of the target biological reasoning model. In some embodiments, the intermediate representation further allows for the insertion of additional metadata, design annotations, and/or user feedback or comments prior to the final compilation into conditioning inputs. In some embodiments, biological design programs, including those with graphical user interfaces, can interact directly with the intermediate representation. Moreover, the intermediate representation has the additional advantage that it can be easily visualized. For example, a visual representation of the intermediate representation can be rendered where the identified design conditions are shown within the context of the visual representation of the intermediate representation.

At 2809, conditioning input is generated for targeted biological reasoning models. For example, the intermediate representation generated at 2807 is compiled into one or more model-specific input format versions that conform to the architecture and interface requirements of each selected biological reasoning model. In various embodiments, depending on the input modality expected by the target model, the conditioning input can include structured tokens, numerical embedding tensors or vectors, per-residue annotations, span-level constraints, positional masks, and/or global embedding vectors. In some embodiments, the conditioning input corresponds to a conditioning track, such as for the disclosed multi-track biological language reasoning model. As another example, a transformer-based biological reasoning model may require residue-level token tracks for encoding structural constraints, while a diffusion-based biological model may require geometry-aware conditioning inputs defined in three-dimensional space. In some embodiments, multiple conditioning inputs are generated for different models in parallel, enabling comparative design or ensemble workflows. In other embodiments, the compiler optimizes and prioritizes the conditioning content based on model-specific input limits, such as token count thresholds or constraint compatibility. The resulting conditioning input serves to encode the biological design intent, as originally specified in a high-level language by the biological language specification, for the determined biological reasoning models.

In some embodiments, as part of step 2809, a final validation check is performed on the generated conditioning input. For example, the model input format version is validated and optionally annotated for additional analysis prior to sending the generated output to an inference pipeline. The validation step can be performed to ensure that the generated conditioning input conforms to the expected format, dimensional structure, and semantic requirements of the targeted biological reasoning model. For example, the model input format version may be checked for missing tokens, misaligned conditioning tracks, or constraint input values that fall outside biologically plausible ranges or thresholds. In some embodiments, the validated conditioning input is further annotated with metadata, such as version tags, constraint scores, or confidence estimates, among other metadata, to support additional analysis, reproducibility, interpretability, and/or interoperability with other models. The validation step can optionally provide for traceability across compilation passes and inference results.

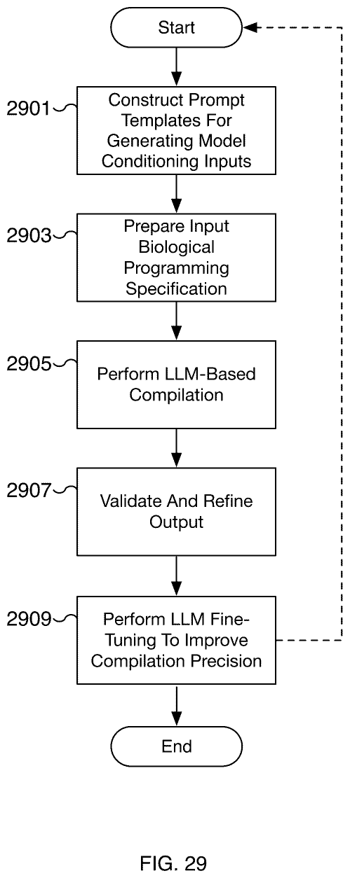

FIG. 29 is a flow chart illustrating an embodiment of a process for compiling a biological programming specification into model-compatible conditioning inputs using a large language model (LLM). Using the process of FIG. 29, an LLM-based compiler is prompted to generate conditioning input for a target biological reasoning model. Moreover, by utilizing contextual prompting and additional fine-tuning techniques, new target generative biological models can be rapidly supported with minimal manual engineering. In various embodiments, the compilation process utilizes the original received biological programming specification and/or focuses on the model-neutral intermediate representation of the specification. For example, by using the model-neutral intermediate representation as an input to the LLM-based compiler, the same biological programming specification can target a wide variety of different models, including new and evolving models still in development. This flexible and scalable approach allows for long-term adaptability and support for diverse biological design workflows. In some embodiments, the process of FIG. 29 is performed at 2601 and/or 2603 of FIG. 26, at 2701, 2703, and/or 2705 of FIG. 27, and/or during the various steps of the process of FIG. 28 in response to a received biological programming specification targeting one or more biological reasoning models. In some embodiments, the process of FIG. 29 is performed at 703 and/or 705 of FIG. 7 by a biological language model service such as biological language model service 111 of FIG. 1 and/or biological language model service 201 of FIG. 2. In some embodiments, one of the targeted biological reasoning models is a multi-track biological language reasoning model and corresponds to biological language model 303 of FIG. 3, biological language model module 401 of FIG. 4, and/or multi-track biological protein language model 503 of FIG. 5.

At 2901, prompt templates are constructed to guide the LLM in generating conditioning inputs for targeted biological reasoning models. In various embodiments, these constructed prompt templates define the structure, content, and formatting of the instructions provided to the LLM, ensuring consistent and accurate translation of the biological programming specification into model-compatible inputs. The prompt templates may include natural language instructions, schema definitions, support for few-shot prompting including example input-output pairs, a detailed and/or extensive dataset of example input-output pairs, documentation of the biological programming language syntax, and/or metadata describing the target model's input format requirements. For example, a prompt template might present an example biological program alongside a correctly formatted input conditioning track for a multi-track protein language model, allowing the LLM to generalize the transformation pattern. In some embodiments, the prompt templates are customized based on the model type, input modality, and/or category of biological constraints, such as based on structural, functional, and/or geometric constraints, among other constraints. The contextual prompt templates can be continuously refined, for example, to improve the generalization ability of the LLM during the compilation process, by using feedback from past compilation results. In some embodiments, the prompt templates are further generated to include domain-specific constraints, restrictions, and/or preferences.

At 2903, an input biological programming specification is prepared. In various embodiments, a received biological programming specification defines the intended design objectives including design conditions, constraints, and requirements expressed via a high-level biological programming language. The specification may be composed manually by a user, such as through a text-based interface, selected and/or manipulated via a graphical user interface, generated programmatically, generated via an LLM such as via voice and/or chat prompts, and/or via back-and-forth conversations, and/or created via another appropriate technique. In some scenarios, the input specification is written directly in the syntax of the biological programming language. In some embodiments, the design objectives are provided, such as by a user, in a natural language format that is automatically converted into a structured program, such as a biological programming specification or directly to an intermediate representation. For example, a user may specify that a protein must include a binding motif within a particular residue range, exhibit certain symmetry properties, and remain solvent-exposed at a particular region. In some embodiments, the input specification is provided directly in the format of the intermediate representation, such as when the input is generated by an application service. In various embodiments, the input biological programming specification includes additional metadata, such as an author identifier, a version number, a project scenario identifier, a timestamp, resource limitations, a provided resource budget, and/or one or more target biological reasoning models. The prepared biological programming specification is provided as one of the inputs to the LLM-based compilation process. In some embodiments, the specification is prepared using one of the prompt templates generated at 2901 and provided as part of the prompting process.

At 2905, an LLM-based compilation is performed. For example, the received biological programming specification, either directly or in the form of an intermediate representation, is provided as input to an LLM that has been configured and/or fine-tuned to function as a compiler for the targeted biological reasoning models. The LLM-based compiler processes the structured input describing design conditions in conjunction with one or more of the prompt templates prepared at step 2901. Example conditions interpreted by the LLM can relate to function, stability, developability, immunogenicity, symmetries of structure, symmetries of amino acid sequences, structure templates including relative positions of atoms within a subset of residues, portions of a biological object which are surface exposed, secondary structure on portions of proteins, hydrophobic amino acid properties of proteins including the quantity of hydrophobic amino acids, the globularity of a portion of a protein, functional specificity for a portion of a biological object, molecular interactions and interfaces including protein to protein interactions and/or interfaces, small molecular interactions, deoxyribonucleic acid (DNA)/ribonucleic acid (RNA) interactions and binding properties, motif and active site scaffolding, and/or post translational modifications, among other conditions. Along with the provided structured input, the prompting can include few-shot examples, contextual instructions, documentation, and domain-specific context to translate the high-level biological design specification and/or the intermediate representation into one or more model-compatible conditioning inputs.

In various embodiments, the LLM-based compiler interprets the programmatic elements describing design constraints and generates properly formatted output compatible as input for target biological reasoning models. For instance, the LLM can interpret and convert the provided conditions, and depending on the input modality expected by a target model, the conditioning input can include structured tokens, numerical embedding tensors or vectors, per-residue annotations, span-level constraints, positional masks, and/or global embedding vectors, among other conditioning input types. In some embodiments, the converted conditioning input corresponds to a conditioning track, such as for the disclosed multi-track biological language reasoning model. Although the input source may be model independent, the generated output is model-specific. For instance, the LLM-based compiler may generate per-residue conditioning input for a protein folding model, global tensors for a diffusion-based model, and/or a conditioning track for a multi-track biological language reasoning model. In some embodiments, the compilation process involves generating multiple candidate outputs, ranking and/or scoring the outputs based on alignment with the original design intent, resolving conflicts and/or inconsistent constraints, and/or validating the intermediate representation before finalizing the conditioning inputs.

In some embodiments, the LLM-based compilation step leverages the model's contextual reasoning abilities to adaptively handle the conditioning inputs. For example, the model, such as the disclosed multi-track biological language reasoning model, can undergo dedicated training to accept the conditioning inputs including even when certain input data may be ambiguous, masked, or include novel constraints. Even in scenarios where the model may not undergo specific training for conditioning inputs, unlike traditional approaches, the LLM-based compilation process allows for significantly improved rapid prototyping and scalable deployment of complex biological designs, while minimizing the need for manual rule engineering or hardcoded compilers.

At 2907, the LLM-based compilation output is validated and refined. For example, the generated conditioning input produced by the LLM-based compiler is checked to ensure that it meets the requirements and limitations of the target biological reasoning model. This validation step may further involve checking that the output conforms to expected formats, such as correct dimensionality of tensors, valid token vocabularies, consistent residue indexing, and/or alignment between structure and sequence annotations and/or is biologically plausible. In some embodiments, the validation process is used to catch and resolve design issues, such as missing constraints, invalid value ranges, and/or conditioning inputs that conflict with one another. For example, a constraint intended for a geometry-based model may be flagged if it includes sequence-only conditioning features that cannot be interpreted in 3D space. In some embodiments, the validation process is further used to notify and/or enforce ethical or safety guidelines, such as to prevent or limit the generation of potentially harmful or unauthorized biological outputs. The validation can be performed prior to outputting the conditioning input to prevent unsafe or ethically problematic instructions from ever reaching the generative backend.

In some embodiments, the refinement performed at 2907 includes additional post-processing to clean up, normalize, and/or reformat the output. In certain embodiments, the output is iteratively improved based on scoring metrics, constraint fulfillment analysis, and/or feedback from simulated and/or dry-run inference passes with the target biological reasoning model. Where discrepancies and/or inconsistencies are detected, the LLM may be re-prompted with additional context, provide suggested modifications to the user, and/or apply fallback heuristics including fallback approaches learned from previous compilation passes.

At 2909, LLM fine-tuning is performed to improve the compilation process. For example, optional tuning, including fine-tuning, can be applied to the LLM to improve the compilation results. In some embodiments, training data composed of biological programming specifications paired with validated conditioning inputs for specific biological reasoning models is used for additional tuning. Moreover, the results from compilation can be analyzed and used to improve the compilation process, including to refine and/or modify the biological programming specification and/or retry the generation of conditioning input from the biological programming specification and/or associated intermediate representation. The additional training allows the model to learn domain-specific mappings and improve the translation accuracy when converting design constraints into model-compatible formats. For example, fine-tuning can help teach the LLM how to generate properly aligned per-residue token tracks for a protein folding model or how to prioritize geometric constraints when preparing inputs for a spatial reasoning model. In some embodiments, the fine-tuning dataset includes examples where the original compilation attempts failed, along with the corrected outputs, allowing the LLM compiler to learn from past errors. The process may also include active learning loops where compilation outputs are scored, reviewed, and used to further refine the compiler's (and/or a target model's) performance and precision.

In certain embodiments, reinforcement learning (RL) and/or other training techniques are also used to further enhance the LLM's ability to generate high-quality conditioning inputs. The LLM-based compiler can be rewarded based on how well the generated inputs satisfy target constraints, minimize conflicts, and/or lead to successful inference results from downstream target models. For example, the RL reward function may incorporate metrics such as biological design goal metrics including metrics related to structural, functional, biophysical, or biochemical objectives. Specific biological design goal metrics may include a minimum predicted stability score, a target binding affinity to a known antigen, an expression likelihood above a defined threshold, function quality, experimental results from wet laboratory synthesis, a desired structural similarity to a biological reference, and/or overall and/or component-based success rates in satisfying design goals. Over time, the provided feedback from model outputs results in enhanced compiler precision. Moreover, by combining supervised fine-tuning with RL-based optimization, the LLM-based compilation process becomes more robust, adaptive, and capable of handling complex biological programming tasks across a range of models and design objectives.

As shown in FIG. 29, a dashed line points from step 2909 to the start of the process of FIG. 29. This dashed line indicates a feedback mechanism for improving the compilation process for the current and/or future compilation passes and/or biological programs. Although the dashed line directs back to the start of the process of FIG. 29, the feedback can be directed to one or more of the steps of FIG. 29, such as to step 2901 to improve the prompt templates construction step, step 2903 to improve the input preparation step, step 2905 to improve the compilation step, and/or step 2907 to improve the validation and refinement step. For instance, the feedback can be used to modify the received biological programming specification to improve the compilation output. As another example, the feedback can be used to regenerate new conditioning input that more accurately reflects the design goals.

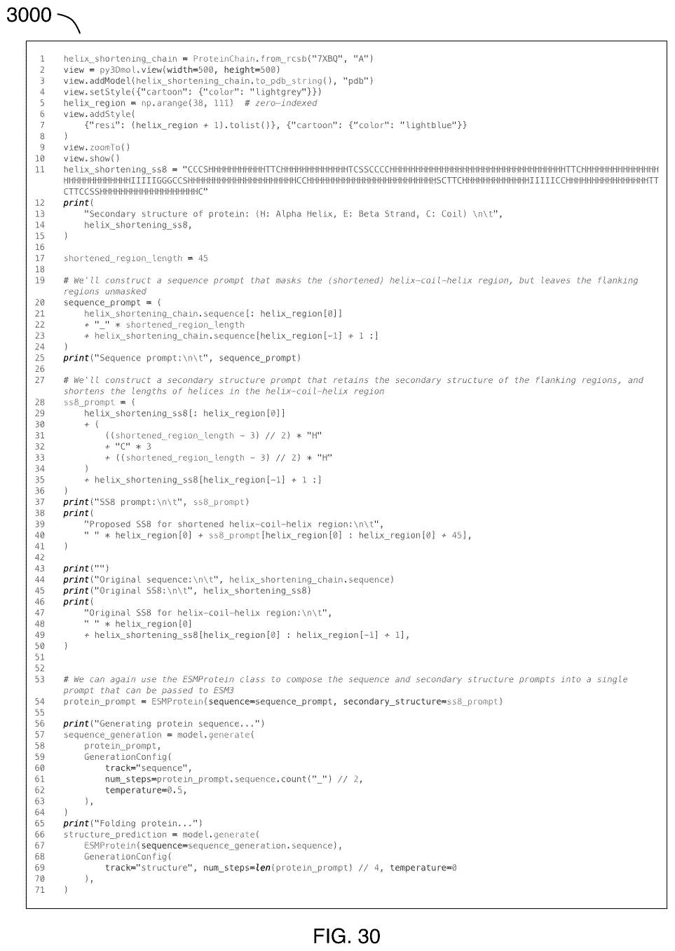

FIG. 30 is a diagram illustrating an embodiment of an excerpt from a biological programming specification for generating a protein design using a biological reasoning model. In the example shown, biological programming specification excerpt 3000 is a portion of a biological program and includes design conditions described in accordance with a biological programming language. The corresponding complete biological program can be compiled to input conditions for a targeted biological reasoning model. For example, the corresponding biological program can be compiled into a conditioning input, such as a conditioning track, for the disclosed multi-track biological language reasoning model and/or conditioning input in a format compatible with another biological reasoning model. In some embodiments, the biological program associated with biological programming specification excerpt 3000 is compiled and used to generate candidate biological designs using the processes described in FIGS. 25-29. In some embodiments, the generative multi-track biological language reasoning model is and/or corresponds to biological language model 303 of FIG. 3, biological language model module 401 of FIG. 4, and/or multi-track biological protein language model 503 of FIG. 5.

In the example shown, biological programming specification excerpt 3000 includes programming constraints that are used to shorten a helix-coil-helix region (residues 39-111) in a protein structure. The helix-coil-helix region in the original protein is 73 residues long and is loaded from entry 7XBQ of the RCSB Protein Data Bank. The expressed design goal shown in biological programming specification excerpt 3000 is to shorten the region to 45 residues using conditions that specify secondary structure editing. Other conditioning input includes a provided partial sequence and secondary structure. In various embodiments, using the techniques disclosed herein, the complete biological program of biological programming specification excerpt 3000 is compiled and the conditioning input is provided to a biological reasoning model. Although not shown in FIG. 30, in various embodiments, the generated structure can be visualized, and the visualization can be initiated via the biological programming language. For example, the generated structure can be visualized alongside the original structure from which the motif was drawn allowing the user to visual confirm the shorten region in a generated protein design.

In the example shown, chain “A” of the protein with the PDB identifier “7XBQ” is retrieved from the RCSB Protein Data Bank using a call to ProteinChain.from_rcsb( . . . ) and specifying as arguments the PDB identifier and corresponding chain identifier, respectively. The retrieved chain is stored in the variable “helix_shortening_chain”. Calls are made to visualize the retrieved chain and, in particular, residues 39 to 111, corresponding to the helix-coil-helix region of the retrieved protein. A representation of the secondary structure of the retrieved protein chain for the helix-coil-helix region is created using the variable “helix_shortening_ss8” with encodings “H” for alpha helix, “E” for beta strand, and “C” for coil.

Next, the target length is set to 45 residues by setting the variable “shortened_region_length” to the design goal length of 45. A sequence prompt (variable “sequence_prompt”) is then constructed by masking the central region with the mask symbol “_”. The flanking regions, or ends of the central region found before and after the helix-coil-helix, are retained and left unmasked. The masking condition allows the target biological reasoning model to fill in or infer the masked region. A corresponding secondary structure prompt (variable “ss8_prompt”) is constructed to go along with the masked protein sequence. The secondary structure prompt retains the secondary structure of the flanking regions but shortens the lengths of the helices in the helix-coil-helix region. This desired shortened length is specified based on, and using, the design condition variable “shortened_region_length”. The desired secondary structure for the shortened helix-coil-helix region is specified with the encodings “H” for helix segments (on both ends but reduced in size compared to the original protein chain) and “C” for coil (in the middle but fixed to 3 residues (“C”*3)). Both constructed prompts (variables “sequence_prompt” and “ss8_prompt”) are created using string manipulation techniques, allowing the flanking regions to be joined at each end of a central region using string-type operations. The two prompts are then combined into a single prompt that can be passed to the biological reasoning model.

In the example shown, a protein design prompt (variable “protein_prompt”) is created using the ESMProtein class, with two arguments: a sequence argument (set to the value of “sequence_prompt”) and a secondary_structure argument (set to the value of “ss8_prompt”). The biological reasoning model is then used to iteratively decode a protein sequence, based on the prompt “protein_prompt”, and to predict the corresponding structure of the decoded sequence. This two-step approach begins by generating the candidate protein sequence, which involves generating a configuration for the biological reasoning model using GenerationConfig( . . . ) and specifying the desired track (“sequence”), number of steps, and temperature. In the example shown, the number of steps (“num_steps”) is configured based on the number of masked positions in the sequence. The model.generate( . . . ) call then generates the protein sequence (storing the result in the variable “sequence_generation”) using the created prompt (“protein_prompt”) and the generated sequence track configuration. Once the sequence is generated, the second step, structure prediction, is performed. The biological reasoning model is reconfigured to predict the corresponding structure by generating a new configuration for the model using another GenerationConfig( . . . ) call and specifying the desired track (“structure”), number of steps, and temperature. The model.generate( . . . ) call is used to fold the candidate protein design (storing the result in the variable “structure_prediction”) using the generated protein sequence (“sequence_generation”) and the generated structure track configuration. Although not shown in FIG. 30, the predicted structure of the protein chain result can be visualized.

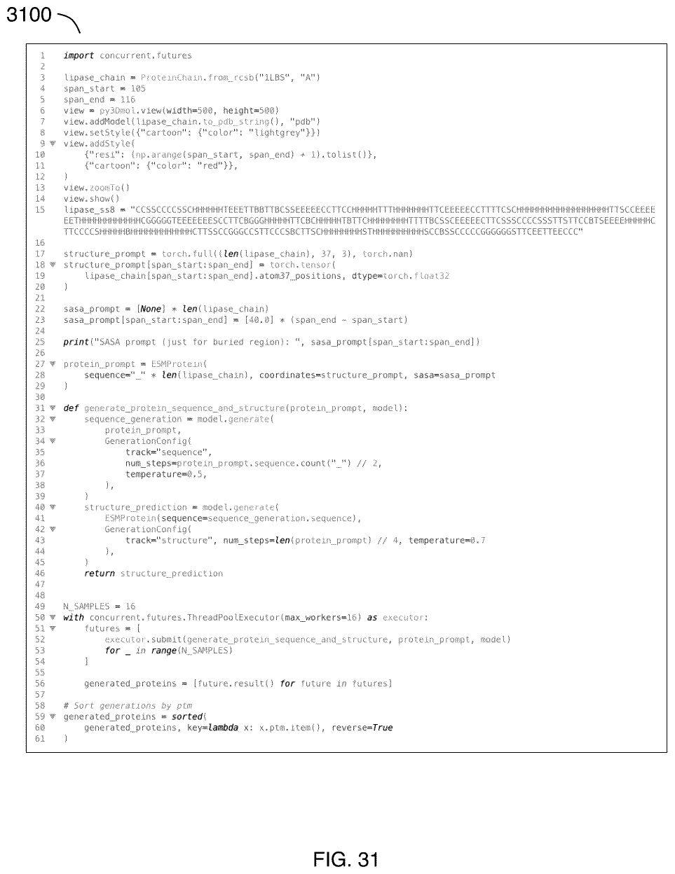

FIG. 31 is a diagram illustrating an embodiment of an excerpt from a biological programming specification for generating a protein design using a biological reasoning model. In the example shown, biological programming specification excerpt 3100 is a portion of a biological program and includes design conditions described in accordance with a biological programming language. The corresponding complete biological program can be compiled to input conditions for a targeted biological reasoning model. For example, the corresponding biological program can be compiled into a conditioning input, such as a conditioning track, for the disclosed multi-track biological language reasoning model and/or conditioning input in a format compatible with another biological reasoning model. In some embodiments, the biological program associated with biological programming specification excerpt 3100 is compiled and used to generate candidate biological designs using the processes described in FIGS. 25-29. In some embodiments, the generative multi-track biological language reasoning model is and/or corresponds to biological language model 303 of FIG. 3, biological language model module 401 of FIG. 4, and/or multi-track biological protein language model 503 of FIG. 5.

In the example shown, biological programming specification excerpt 3100 includes programming constraints that are used to perform Solvent Accessible Surface Area (SASA) editing. The design goal of the corresponding biological program is to expose a buried helix within a protein (PDB ID: 1LBS, chain A) by reconditioning its surface accessibility. The source protein, 1LBS, has an alternating alpha-beta sandwich fold, with a buried helix in the center. In the example shown, biological programming specification excerpt 3100 includes conditions to generate a protein sequence that surface-exposes a previously buried helix, while also preserving the structural and sequence context. The conditions specifically require high SASA values for the residues in the buried helix, causing the model to generate a protein design that exposes the helix to the surface of the protein. For improved results, multiple generations of candidate designs are sampled. In various embodiments, the candidates can be sorted by the generation with the highest predicted TM-score (pTM). Although not shown in FIG. 31, in various embodiments, the top scoring generations can be visualized, and the visualization can be initiated via the biological programming language. For example, the top 4 generations by pTM can be visualized alongside with the original protein allowing the user to visual evaluate and/or confirm the generated protein designs.

In the example shown, chain “A” of the protein with the PDB identifier “1LBS” is retrieved from the RCSB Protein Data Bank using a call to ProteinChain.from_rcsb( . . . ) and specifying as arguments the PDB identifier and corresponding chain identifier, respectively. The retrieved chain is stored in the variable “lipase_chain” and the span between residues 105 and 116 is emphasized using the variables “span_start” and “span_end”. Calls are made to visualize the retrieved chain and, in particular, the residues of the specified span, which correspond to a buried helix in the center of an alternating alpha-beta sandwich fold. A representation of the secondary structure of the retrieved protein chain with the buried helix is created using the variable “lipase_ss8” with encodings “C” for coil, “S” for bend, “H” for alpha helix, “T” for beta turn, “E” for beta strand, “B” for beta bridge, and “G” for 3₁₀ helix. In some embodiments, a standard ss8 classification encoding is used, although alternative encodings can be appropriate as well.

Next, a multimodal prompt is created by first constructing a structure prompt (variable “structure_prompt”) using the retrieved lipase chain. This prompts the biological reasoning model to generate a candidate that contains the same helix. A corresponding SASA requirement is expressed by creating an SASA prompt (variable “sasa_prompt”) that has high SASA values (e.g., 40.0) for the span corresponding to the buried helix. This design goal prompts the biological reasoning model to expose the helix to the surface of the generated protein design candidate. A single prompt (variable “protein prompt”) with multiple conditions is created using: (1) a fully masked protein sequence argument (“sequence”) using mask symbol “_” that has the same length as the retrieved protein chain (“lipase_chain”), (2) a coordinates argument (“coordinates”) set to the structure prompt (“structure_prompt”), and (3) an SASA (“sasa”) argument set to the SASA prompt (“sasa_prompt”). A function generate_protein_sequence_and_structure( . . . ) is defined to take a protein prompt and model as arguments, and iteratively decode a protein sequence and generate a corresponding structure using the specified model and protein prompt. The biological program specification then instructs the biological reasoning model to generate 16 samples using the constructed prompt (variable “protein prompt”). The samples are then sorted using a sorted( . . . ) call, with the generated samples, a key, and a reverse parameter as arguments. In the example shown, the key argument (“key”) used for sorting specifies using a predicted TM-score (“ptm”). In the example shown, the design goals specify generating 16 different candidate protein designs.

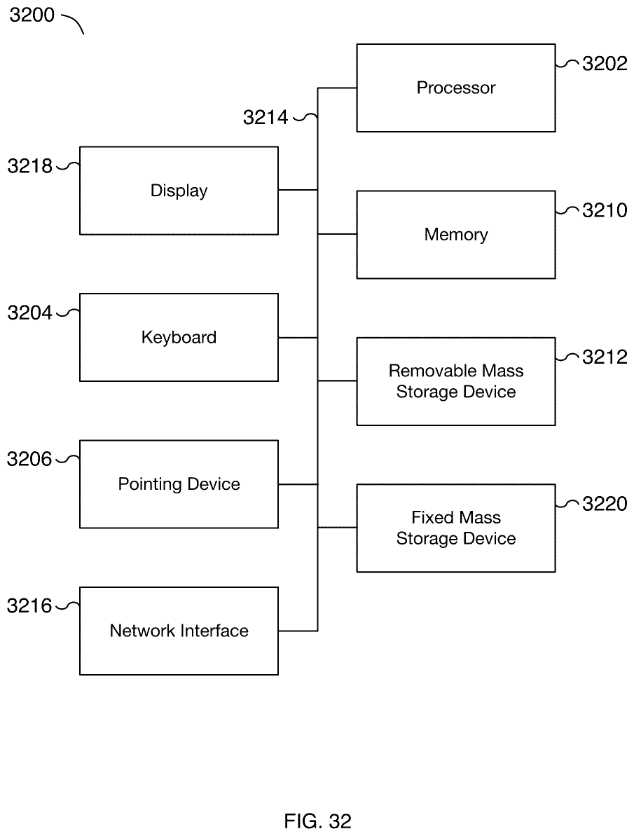

FIG. 32 is a functional diagram illustrating a programmed computer system for performing biological language reasoning. In some embodiments, the biological language reasoning is performed at least in part at the direction of a biological language program. As will be apparent, other computer system architectures and configurations can be utilized for performing biological language reasoning including those with one or more graphical processing units (GPUs). Examples of computer system 3200 include clients 101, 103, and 105 of FIG. 1, one or more computers of biological language model service 111 of FIG. 1, and/or one or more computers of biological language model service 201 of FIG. 2. Additional examples of computer system 3200 include one or more computers used to implement biological language model 303 of FIG. 3, biological language model module 401 of FIG. 4, multi-track biological protein language model 503 of FIG. 5, transformer block with a geometric attention 603 of FIG. 6, biological structure tokenizer module 1201 of FIG. 12, structure tokenizer 1300 of FIG. 13, structure encoder 1403 of FIG. 14, geometric reasoning block 1503 of FIG. 15, geometric attention block 1903 of FIG. 19, and/or geometric attention block 2003 of FIG. 20. Computer system 3200, which includes various subsystems as described below, includes at least one microprocessor subsystem (also referred to as a processor or a central processing unit (CPU)) 3202. For example, processor 3202 can be implemented by a single-chip processor or by multiple processors. In some embodiments, processor 3202 is a general purpose digital processor that controls the operation of the computer system 3200. Using instructions retrieved from memory 3210, the processor 3202 controls the reception and manipulation of input data, and the output and display of data on output devices (e.g., display 3218). In various embodiments, one or more instances of computer system 3200 can be used to implement at least portions of the processes of FIGS. 7-11, 16-18, and/or 21-29 and to evaluate the biological programming specifications corresponding to the diagrams of FIGS. 30 and/or 31.

Processor 3202 is coupled bi-directionally with memory 3210, which can include a first primary storage, typically a random access memory (RAM), and a second primary storage area, typically a read-only memory (ROM). As is well known in the art, primary storage can be used as a general storage area and as scratch-pad memory, and can also be used to store input data and processed data. Primary storage can also store programming instructions and data, in the form of data objects and text objects, in addition to other data and instructions for processes operating on processor 3202. Also as is well known in the art, primary storage typically includes basic operating instructions, program code, data and objects used by the processor 3202 to perform its functions (e.g., programmed instructions). For example, memory 3210 can include any suitable computer-readable storage media, described below, depending on whether, for example, data access needs to be bi-directional or unidirectional. For example, processor 3202 can also directly and very rapidly retrieve and store frequently needed data in a cache memory (not shown).

A removable mass storage device 3212 provides additional data storage capacity for the computer system 3200, and is coupled either bi-directionally (read/write) or unidirectionally (read only) to processor 3202. For example, storage 3212 can also include computer-readable media such as magnetic tape, flash memory, PC-CARDS, portable mass storage devices, holographic storage devices, and other storage devices. A fixed mass storage 3220 can also, for example, provide additional data storage capacity. The most common example of mass storage 3220 is a hard disk drive. Mass storages 3212, 3220 generally store additional programming instructions, data, and the like that typically are not in active use by the processor 3202. It will be appreciated that the information retained within mass storages 3212 and 3220 can be incorporated, if needed, in standard fashion as part of memory 3210 (e.g., RAM) as virtual memory.

In addition to providing processor 3202 access to storage subsystems, bus 3214 can also be used to provide access to other subsystems and devices. As shown, these can include a display monitor 3218, a network interface 3216, a keyboard 3204, and a pointing device 3206, as well as an auxiliary input/output device interface, a sound card, speakers, and other subsystems as needed. For example, the pointing device 3206 can be a mouse, stylus, track ball, or tablet, and is useful for interacting with a graphical user interface.

The network interface 3216 allows processor 3202 to be coupled to another computer, computer network, or telecommunications network using a network connection as shown. For example, through the network interface 3216, the processor 3202 can receive information (e.g., data objects or program instructions) from another network or output information to another network in the course of performing method/process steps. Information, often represented as a sequence of instructions to be executed on a processor, can be received from and outputted to another network. An interface card or similar device and appropriate software implemented by (e.g., executed/performed on) processor 3202 can be used to connect the computer system 3200 to an external network and transfer data according to standard protocols. For example, various process embodiments disclosed herein can be executed on processor 3202, or can be performed across a network such as the Internet, intranet networks, or local area networks, in conjunction with a remote processor that shares a portion of the processing. Additional mass storage devices (not shown) can also be connected to processor 3202 through network interface 3216.

An auxiliary I/O device interface (not shown) can be used in conjunction with computer system 3200. The auxiliary I/O device interface can include general and customized interfaces that allow the processor 3202 to send and, more typically, receive data from other devices such as microphones, touch-sensitive displays, transducer card readers, tape readers, voice or handwriting recognizers, biometrics readers, cameras, portable mass storage devices, and other computers.

In addition, various embodiments disclosed herein further relate to computer storage products with a computer readable medium that includes program code for performing various computer-implemented operations. The computer-readable medium is any data storage device that can store data which can thereafter be read by a computer system. Examples of computer-readable media include, but are not limited to, all the media mentioned above: magnetic media such as hard disks, floppy disks, and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code, as produced, for example, by a compiler, or files containing higher level code (e.g., script) that can be executed using an interpreter.

The computer system shown in FIG. 32 is but an example of a computer system suitable for use with the various embodiments disclosed herein. Other computer systems suitable for such use can include additional or fewer subsystems. In addition, bus 3214 is illustrative of any interconnection scheme serving to link the subsystems. Other computer architectures having different configurations of subsystems can also be utilized.

Although the foregoing embodiments have been described in some detail for purposes of clarity of understanding, the invention is not limited to the details provided. There are many alternative ways of implementing the invention. The disclosed embodiments are illustrative and not restrictive.

1. A method, comprising:
receiving a biological programming specification that identifies at least one protein design condition in accordance with a biological programming language;
using a machine learning model to convert the biological programming specification to a model input format version for a biological reasoning model; and
using the model input format version as a conditioning input for the biological reasoning model to generate a protein design having the at least one protein design condition.

2. The method of claim 1, wherein using the machine learning model to convert the biological programming specification to the model input format version for the biological reasoning model includes identifying one or more conditions specified by the biological programming specification, and wherein the identified one or more conditions includes the at least one protein design condition.

3. The method of claim 2, further comprising identifying one or more invalid or is incompatible conditions among the identified one or more conditions.

4. The method of claim 2, wherein the identified one or more conditions are each associated with a corresponding node of a syntax tree.

5. The method of claim 4, wherein the syntax tree includes terminal nodes and non-terminal nodes, and wherein a specific condition of the identified one or more conditions that is associated with a non-terminal node of the syntax tree applies to child nodes of the non-terminal node.

6. The method of claim 1, wherein using the machine learning model to convert the biological programming specification to the model input format version for the biological reasoning model includes converting the biological programming specification to an intermediate representation and converting the intermediate representation to the model input format version, wherein the intermediate representation is compatible with a second biological reasoning model different from the biological reasoning model.

7. The method of claim 6, wherein the intermediate representation includes a syntax tree, a structured object graph, or a prioritized list of normalized conditions.

8. The method of claim 6, further comprising providing a visual representation of the intermediate representation and the at least one protein design condition within the context of the visual representation of the intermediate representation.

9. The method of claim 1, wherein the biological reasoning model is a multi-track model; and wherein the conditioning input for the biological reasoning model corresponds to a conditioning track of the multi-track model.

10. The method of claim 1, wherein the at least one protein design condition is associated with at least one of: a stability property of a protein, a developability property of a protein, a immunogenicity property of a protein, a functional specificity of a protein, a protein to protein interaction, a small molecule interaction, a deoxyribonucleic acid (DNA) interaction, a ribonucleic acid (RNA) interaction, a motif scaffolding, an active site scaffolding, a post translational modification, structure symmetry, symmetries of an amino acid sequence, a structure template, a surface exposed portion of a protein, a secondary structure of a portion of a protein, a hydrophobic property of a protein, or a globularity property of a protein.

11. The method of claim 1, wherein using the machine learning model to convert the biological programming specification to the model input format version for the biological reasoning model includes providing the machine learning model with documentation of the biological programming language and one or more biological program and conditioning input pairs for the biological reasoning model.

12. A system, comprising:
one or more processors configured to:
receive a biological programming specification that identifies at least one protein design condition in accordance with a biological programming language;
use a machine learning model to convert the biological programming specification to a model input format version for a biological reasoning model; and
use the model input format version as a conditioning input for the biological reasoning model to generate a protein design having the at least one protein design condition; and
a memory coupled to the one or more processors, wherein the memory is configured to provide the one or more processors with instructions.

13. The system of claim 12, wherein to use the machine learning model to convert the biological programming specification to the model input format version for the biological reasoning model includes to identify one or more conditions specified by the biological programming specification, and wherein the identified one or more conditions includes the at least one protein design condition.

14. The system of claim 13, wherein the one or more processors are further configured to:
identify one or more invalid or incompatible conditions among the identified one or more conditions.

15. The system of claim 12, wherein to use the machine learning model to convert the biological programming specification to the model input format version for the biological reasoning model includes to convert the biological programming specification to an intermediate representation and to convert the intermediate representation to the model input format version, wherein the intermediate representation is compatible with a second biological reasoning model different from the biological reasoning model.

16. The system of claim 12, wherein the at least one protein design condition is associated with at least one of: a stability property of a protein, a developability property of a protein, a immunogenicity property of a protein, a functional specificity of a protein, a protein to protein interaction, a small molecule interaction, a deoxyribonucleic acid (DNA) interaction, a ribonucleic acid (RNA) interaction, a motif scaffolding, an active site scaffolding, a post translational modification, structure symmetry, symmetries of an amino acid sequence, a structure template, a surface exposed portion of a protein, a secondary structure of a portion of a protein, a hydrophobic property of a protein, or a globularity property of a protein.

17. The system of claim 12, wherein using the machine learning model to convert the biological programming specification to the model input format version for the biological reasoning model includes providing the machine learning model with documentation of the biological programming language and one or more biological program and conditioning input pairs for the biological reasoning model.

18. A method, comprising:
identifying a condition defined by a biological programming language specification;
generating a reference biological language program that includes the defined condition;
identifying one or more training example proteins that conform to the condition defined by the biological programming language specification; and
training a biological language reasoning model using the generated reference biological language program as an input and the identified one or more training example proteins as outputs.

19. The method of claim 18, wherein identifying the one or more training example proteins that conform to the condition defined by the biological programming language specification further includes:
determining a metric value associated with the condition for a plurality of candidate proteins; and
based on the determined metric value, including a protein of the plurality of candidate proteins in a training data set.

20. The method of claim 18, wherein the condition defined by the biological programming language specification is associated with at least one of: a stability property of a protein, a developability property of a protein, a immunogenicity property of a protein, a functional specificity of a protein, a protein to protein interaction, a small molecule interaction, a deoxyribonucleic acid (DNA) interaction, a ribonucleic acid (RNA) interaction, a motif scaffolding, an active site scaffolding, a post translational modification, structure symmetry, symmetries of an amino acid sequence, a structure template, a surface exposed portion of a protein, a secondary structure of a portion of a protein, a hydrophobic property of a protein, or a globularity property of a protein.
